SEQUENCE LISTING (78 sequences)

SEQ ID NO: 1 - DNA, 739 bp, Homo sapiens
atggcattgtcgatgccactgaacaagttgaaggaggaagacaaagagcccctccttgagctctgggtcaaggctgtcagtgatggtgaaagcacaggaatctgccttttttcccagagattcctcatgattctttggctcaaaggagttgtcttcagtgtcacaactgttgatctgaaaaggaaacctgcagatctgcaaaacaaggctcctgggaaccacccaccacttataacttcaacagtgaagtcaaataagattgaggaagctcctgaagaagtcttatgtcctcccaagtacttaaagctttcaccaaaacacccagaatcaaatactgctggaatggacatctttgccaaattctctgcatacatcaagaattcaaggccagaggttaatgaagcattagtgaagcatctcttaaaaaccctgcagaaaatggaatatctgaattctcctctccctgatgaaattgatgaaaatagcatgcaggacactaagttttctacacataaatttctgaatggcaataaaatggcattagctgattgccatctgctgcccaaactgcatattgtcaaaaaaaaagaaaaatatagaaaatataaaaatatagaaaaaaaaggaatgactggcatctggagatacctaacgaatacaagtagtagggatatgttcaacaatacctgtcccaatgataaagagattgaaatagcagcagaaacagttaatgtagtaa

SEQ ID NO: 2 - protein, 246 aa, Homo sapiens
MALSMPLNKLKEEDKEPLLELWVKAVSDGESTGICLFSQRFLMILWLKGVVFSVTTVDLKRKPADLQNKAPGNHPPLITSTVKSNKIEEAPEEVLCPPKYLKLSPKHPESNTAGMDIFAKFSAYIKNSRPEVNEALVKHLLKTLQKMEYLNSPLPDEIDENSMQDTKFSTHKFLNGNKMALADCHLLPKLHIVKKKEKYRKYKNIEKKGMTGIWRYLTNTSSRDMFNNTCPNDKEIEIAAETVNVV

SEQ ID NO: 3 - DNA, 550 bp, Homo sapiens
tctgaggacacagccacactcttgtcatgccattgcccttctattctttccttataacatcatgtaagagggcacagcatgtttcccatgctggaccctgctctgctcactccacacaccttctgacacccaccatggacactgttcagcaactggaagaaagagggcacctgatggacagcaaaggctttgatgaaaataaatacatgaaggaactaggagtgggactagccctctgcgaaaaaaagggtgctatggccaaaaaagattgtattagcttttttgatggcaaaaacctcaccataaaaatggagagtactttaaaatcatacagttttctcacactcaggggagggaaattcaaagaaactacaggtgacggcagaaaaactcagacttgcacctttacatatggcacattggttcgacatcagaagtggaatggaaaggaaggcaaaataagaaaattgaaagacaggaaattagtggtggactgcatcataaacaatgtcacctgtactcagatctatgaaaaagtagaataaaaact

SEQ ID NO: 4 - protein, 172 aa, Homo sapiens
MPLPFYSFLITSCKRAQHVSHAGPCSAHSTHLLTPTMDTVQQLEERGHLMDSKGFDENKYMKELGVGLALCEKKGAMAKKDCISFFDGKNLTIKMESTLKSYSFLTLRGGKFKETTGDGRKTQTCTFTYGTLVRHQKWNGKEGKIRKLKDRKLVVDCIINNVTCTQIYEKVE

SEQ ID NO: 5 - DNA, 915 bp, Homo sapiens
atgctgccgccgccgcggcccgcagctgccttggcgctgcctgtgctcctgctactgctggtggtgctgacgccgcccccgaccggcgcaaggccatccccaggcccagattacctgcggcgcggctggatgcggctgctagcggagggcgagggctgcgctccctgccggccagaagagtgcgccgcgccgcggggctgcctggcgggcagggtgcgcgacgcgtgcggctgctgctgggaatgcgccaacctcgagggccagctctgcgacctggaccccagtgctcacttctacgggcactgcggcgagcagcttgagtgccggctggacacaggcggcgacctgagccgcggagaggtgccggaacctctgtgtgcctgtcgttcgcagagtccgctctgcgggtccgacggtcacacctactcccagatctgccgcctgcaggaggcggcccgcgctcggcccgatgccaacctcactgtggcacacccggggccctgcgaatcggggccccagatcgtgtcacatccatatgacacttggaatgtgacagggcaggatgtgatctttggctgtgaagtgtttgcctaccccatggcctccatcgagtggaggaaggatggcttggacatccagctgccaggggatgacccccacatctctgtgcagtttaggggtggaccccagaggtttgaggtgactggctggctgcagatccaggctgtgcgtcccagtgatgagggcacttaccgctgccttggccgcaatgccctgggtcaagtggaggcccctgctagcttgacagtgctcacacctgaccagctgaactctacaggcatcccccagctgcgatcactaaacctggttcctgaggaggaggctgagagtgaagagaatgacgattactactag

SEQ ID NO: 6 - protein, 304 aa, Homo sapiens
MLPPPRPAAALALPVLLLLLVVLTPPPTGARPSPGPDYLRRGWMRLLAEGEGCAPCRPEECAAPRGCLAGRVRDACGCCWECANLEGQLCDLDPSAHFYGHCGEQLECRLDTGGDLSRGEVPEPLCACRSQSPLCGSDGHTYSQICRLQEAARARPDANLTVAHPGPCESGPQIVSHPYDTWNVTGQDVIFGCEVFAYPMASIEWRKDGLDIQLPGDDPHISVQFRGGPQRFEVTGWLQIQAVRPSDEGTYRCLGRNALGQVEAPASLTVLTPDQLNSTGIPQLRSLNLVPEEEAESEENDDYY

SEQ ID NO: 7 - DNA, 1299 bp, Homo sapiens
cagcatgagcttcaccactccctccaccttctccaccaactaccagtccctgggctctgtccagccgcccagctatggcacctggccggtcagcagcgcagccagcatctatgcaggcactggggggcttgggtcccagatctccatgtcctgttctaccagtttctggggcggcttggggtctgggggcctggccacagagatggctgggggtctggcagaaatggggggcatccagaatgagaaggagaccatgcaaagcctgaacgaccacctggactacctggacagagtgaggaacctggagaccgagaactggaggctggagagcaaaatccaggagtatctggagaagagaccccatgtcagagactggggccattacttcaagaccatcaaggaactgagggctcagatcttcgcaaatactgtggacaatgtccacatcattctgcagatcgacaatgcccgtcttgctgctgatgacttcagagtcaagtatgagacagagctggccatgcgccagtctgtggagagcaacatccatgggctctgcaaggtcattgatgacaccaatgtcactctgctgcagctggagacagagatgggcgctctcaaggaggagctgctcctcatgaagaagaaccatgaagaggaagtaaaaggcttgcaagtccagattgccaactctgggttggccgtggaggtagatgcccccaaatctcaagtcctcgccaaggtcatggcagacatcagggcccaatatgatgagctgtctcagaagaactcagagaagctaggcaagtactggtctcagcagactgaggagagcaccacagtggtcaccacacactctgccaaggtcagagctgctgagatgacaacggagctgagacgtacagtccagtgcttggagattgacctggactcaatgagaaatctgaagaccagcttggagaacagcctgagggaggtggaggcccgctacgccctgcagatggagcagctcaacagaatcctgctgtacttggagtcaaagctggcacagaactgggcagagggccagcgcaaggtccaggagtacaaggacttgctgaacatcagggtcaagctggaggctgagatcgccacctaccgccgcctgctggaagacagcgagggcctcaatcttggtgatgccctggacagcagcaactccatgcaaaccatccaaaagaccaccacccgccagatagtggatagcaaagtggtgtctgagatcagtgacaccaaagttctgagacattaagccagcagaag

SEQ ID NO: 8 - protein, 427 aa, Homo sapiens [sequence omitted]

SEQ ID NO: 9 - DNA, 2202 bp, Homo sapiens
atgtgggggctcctgctcgccctggccggcttcgcgccggccgtcggcccggctctgggggcgcccaggaactcggtgctgggcctcgcgcagcccgggaccaccaaggtcccaggctcgaccccggccctgcatagcagcccggcacagccgtcggcggagacagctaacacctcagaacagcatgtccggattcgagtcatcaagaagaaaaaggtcattatgaagaagcggaagaagctaactctaactcgccccaccccactggtgactgccgggccccttgtgacccccactccagcagggaccctcgaccccgctgagaaacaagaaccaggctgtcctcctttgggtctggagtccctgcgagtttcagatagccggcttgaggcatccagcagccagtcctttggtcttggaccacaccgaggacggctcaacattcagtcaggcctggaggacggcgatctatatgatggagcctggtgtgctgaggagcaggacgccgatccatggtttcaggtggacgctgggcaccccacccgcttctcgggtgttatcacacagggcaggaactctgtctggaggtatgactgggtcacatcatacaaggtccagttcagcaatgacagtcggacctggtggggaagtaggaaccacagcagtgggatggacgcagtgtttcctgccaattcagacccagaaactccagtgctgaacctcctgccggagccccaggtggcccgcttcattcgcctgctgccccagacctggctccagggaggcgcgccttgcctccgggcagagatcctggcctgcccagtctcagaccccaatgacctattccttgaggcccctgcgtcgggatcctctgaccctctagactttcagcatcacaattacaaggccatgaggaagctgatgaagcaggtacaagagcaatgccccaacattacccgcatctacagcattgggaagagctaccagggcctgaagctgtatgtgatggaaatgtcggacaagcctggggagcatgagctgggtgagcctgaggtgcgctacgtggctggcatgcatgggaacgaggccctggggcgggagttgcttctgctcctgatgcagttcctgtgccatgagttcctgcgagggaacccacgggtgacccggctgctctctgagatgcgcattcacctgctgccctccatgaaccctgatggctatgagatcgcctaccaccggggttcagagctggtgggctgggccgagggccgctggaacaaccagagcatcgatcttaaccataattttgctgacctcaacacaccactgtgggaagcacaggacgatgggaaggtgccccacatcgtccccaaccatcacctgccattgcccacttactacaccctgcccaatgccaccgtggctcctgaaacgcgggcagtaatcaagtggatgaagcggatcccctttgtgctaagtgccaacctccacgggggtgagctcgtggtgtcctacccattcgacatgactcgcaccccgtgggctgcccgcgagctcacgcccacaccagatgatgctgtgtttcgctggctcagcactgtctatgctggcagtaatctggccatgcaggacaccagccgccgaccctgccacagccaggacttctccgtgcacggcaacatcatcaacggggctgactggcacacggtccccgggagtatgaatgacttcagctacctacacaccaactgctttgaggtcactgtggagctgtcctgtgacaagttccctcacgagaatgaattgccccaggagtgggagaacaacaaagacgccctcctcacctacctggagcaggtgcgcatgggcattgcaggagtggtgagggacaaggacacggagcttgggattgctgacgctgtcattgccgtggatgggattaaccatgacgtgaccacggcgtggggcggggattattggcgtctgctgaccccaggggactacatggtgactgccagtgccgagggctaccattcagtgacacggaactgtcgggtcactttgaagaggggccccttcccctgcaatttcgtgctcaccaagactcccaaacagaggctgcgcgagctgctggcagctggggccaaggtgcccccggaccttcgcaggcgcctggagcggctaaggggacagaaggattga

SEQ ID NO: 10 - protein, 733 aa, Homo sapiens
MWGLLLALAGFAPAVGPALGAPRNSVLGLAQPGTTKVPGSTPALHSSPAQPSAETANTSEQHVRIRVIKKKKVIMKKRKKLTLTRPTPLVTAGPLVTPTPAGTLDPAEKQEPGCPPLGLESLRVSDSRLEASSSQSFGLGPHRGRLNIQSGLEDGDLYDGAWCAEEQDADPWFQVDAGHPTRFSGVITQGRNSVWRYDWVTSYKVQFSNDSRTWWGSRNHSSGMDAVFPANSDPETPVLNLLPEPQVARFIRLLPQTWLQGGAPCLRAEILACPVSDPNDLFLEAPASGSSDPLDFQHHNYKAMRKLMKQVQEQCPNITRIYSIGKSYQGLKLYVMEMSDKPGEHELGEPEVRYVAGMHGNEALGRELLLLLMQFLCHEFLRGNPRVTRLLSEMRIHLLPSMNPDGYEIAYHRGSELVGWAEGRWNNQSIDLNHNFADLNTPLWEAQDDGKVPHIVPNHHLPLPTYYTLPNATVAPETRAVIKWMKRIPFVLSANLHGGELVVSYPFDMTRTPWAARELTPTPDDAVFRWLSTVYAGSNLAMQDTSRRPCHSQDFSVHGNIINGADWHTVPGSMNDFSYLHTNCFEVTVELSCDKFPHENELPQEWENNKDALLTYLEQVRMGIAGVVRDKDTELGIADAVIAVDGINHDVTTAWGGDYWRLLTPGDYMVTASAEGYHSVTRNCRVTLKRGPFPCNFVLTKTPKQRLRELLAAGAKVPPDLRRRLERLRGQKD

SEQ ID NO: 11 - DNA, 846 bp, Homo sapiens
cgcagatgctgtggctgctattcctgaccctcccctgcctggggggctccatgtccaagaccccagtgcccgtcccagagaatgacctggtgggcattgtggggggccacaatgcccccccggggaagtggccgtggcaggtcagcctgagggtctacagctaccactgggcctcctgggcgcacatctgtgggggctccctcatccacccccagtgggtgctgactgctgcccactgcattttctggaaggacaccgacccgtccatctaccggatccacgctggggacgtgtatctctacgggggccgggggctgctgaacgtcagccggatcatcgtccaccccaactatgtcactgcggggctgggtgcggatgtggccctgctccagctggtgagccccatgatcggagccgctaatgtcaggacggtcaagctctccccggtctcgctggagctcaccccgaaggaccagtgctgggtgactggctggggagcgatcaggatgttcgagtcgctgccgccgccctaccgcctgcagcaggcgagtgtgcaggtgctggagaacgccgtctgtgagcagccctaccgcaacgcctcagggcacactggcgaccggcagctcatcctggatgacatgctgtgtgccggcagcgagggccgagactcctgtcagggtgactccggcggccctctggtctgcaggctgcgggggtcctggcgcctggtgggggtggtcagctggggctacggctgtaccctgcgggactttcccggcgtctacacccacgtccagatctacgtgctctggatcctgcagcaagtcggggagttgccctgagcag

SEQ ID NO: 12 - protein, 278 aa, Homo sapiens
MLWLLFLTLPCLGGSMSKTPVPVPENDLVGIVGGHNAPPGKWPWQVSLRVYSYHWASWAHICGGSLIHPQWVLTAAHCIFWKDTDPSIYRIHAGDVYLYGGRGLLNVSRIIVHPNYVTAGLGADVALLQLVSPMIGAANVRTVKLSPVSLELTPKDQCWVTGWGAIRMFESLPPPYRLQQASVQVLENAVCEQPYRNASGHTGDRQLILDDMLCAGSEGRDSCQGDSGGPLVCRLRGSWRLVGVVSWGYGCTLRDFPGVYTHVQIYVLWILQQVGELP

SEQ ID NO: 13 - DNA, 2145 bp, Homo sapiens
gatccgggggctcctgtgaccatgccctcttctcgcccgcaggtcggccacgggacctgacgcaacaggatggacgagtcccctgagcctctgcagcagggcagagggccggtgccggtccgacgccagcgcccagcaccccggggtctgcgtgagatgctgaaggccaggctgtggtgcagctgctcgtgcagtgtgctgtgcgtccgggcgctggtgcaggacctgctccccgccacgcgctggctgcgtcagtaccgcccgcgggagtacctggcaggcgacgtcatgtctgggctggtcatcggcatcatcctggtcccgcaggccatcgcctactcattgctggccgggctgcagcccatctacagcctctatacgtccttcttcgccaacctcatctacttcctcatgggcacctcacggcatgtctccgtgggcatcttcagcctgctttgcctcatggtggggcaggtggtggaccgggagctccagctggccggctttgacccctcccaggacggcctgcagcccggagccaacagcagcaccctcaacggctcggctgccatgctggactgcgggcgtgactgctacgccatccgtgtcgccaccgccctcacgctgatgaccgggctttaccaggtcctcatgggcgtcctccggctgggcttcgtgtccgcctacctctcacagccactgctcgatggctttgccatgggggcctccgtgaccatcctgacctcgcagctcaaacacctgctgggcgtgcggatcccgcggcaccaggggcccggcatggtggtcctcacatggctgagcctgctgcgcggcgccgggcaggccaacgtgtgcgacgtggtcaccagcacggtgtgcctggcggtgctgctagccgcgaaggagctctcagaccgctaccgacaccgcctgagggtgccgctgcccacggagctgctggtcatcgtggtggccacactcgtgtcgcacttcgggcagctccacaagcgctttggctcgagcgtggctggcgacatccccacgggtttcatgccccctcaggtcccagagcccaggctgatgcagcgtgtggctttggatgccgtggccctggccctcgtggctgccgccttctccatctcgctggcggagatgttcgcccgcagtcacggctactctgtgcgtgccaaccaggagctgctggctgtgcatcgtggtcacctgcggggggcctgccaaggtgtgggactcccgggctgtggcggatcaccggctgacgcgctggtctgggcaggcacgggcacctgtatgctggtcagcacagaggccgggctgctggctggcgtcatcctctcgctgctcagcctggccggccgcacccaaaagccacgcaccgccctgctggcccgcatcggggacacggccttctacgaggatgccacagagttcgagggcctcgtccctgagcccggcgtgcgggtgttccgctttggggggccgctgtactatgccaacaaggacttcttcctgcagtcactctacagcctcacggggctggacgcagggtgcatggctgccaggaggaaggaggggggctcagagacgggggtcggtgagggaggccctgcccagggcgaggacctgggcccggttagcaccagggctgcgctggtgcccgcagcggccggcttccacacagtggtcatcgactgcgccccgctgctgttcctagacgcagccggtgtgagcacgctgcaggacctgcgccgagactacggggccctgggcatcagcctgctgctagcctgctgcagcccgcctgtgagagacattctgagcagaggaggcttcctcggggagggccccggggacacggctgaggaggagcagctgttcctcagtgtgcacgatgccgtgcagacagcacgagcccgccacagggagctggaggccaccgatgcccatctgtagcagggccaggcctgcccagcagcctctgctccctcctggggacccacagcagacgtctgcaagccactgctgagacccttcccagggaggagccacccaagagctgcactcttgtgccacagctgccctggggaaaccggggaaccccaactgggaaaggaggccctctgatca

SEQ ID NO: 14 - protein, 633 aa, Homo sapiens
MDESPEPLQQGRGPVPVRRQRPAPRGLREMLKARLWCSCSCSVLCVRALVQDLLPATRWLRQYRPREYLAGDVMSGLVIGIILVPQAIAYSLLAGLQPIYSLYTSFFANLIYFLMGTSRHVSVGIFSLLCLMVGQVVDRELQLAGFDPSQDGLQPGANSSTLNGSAAMLDCGRDCYAIRVATALTLMTGLYQVLMGVLRLGFVSAYLSQPLLDGFAMGASVTILTSQLKHLLGVRIPRHQGPGMVVLTWLSLLRGAGQANVCDVVTSTVCLAVLLAAKELSDRYRHRLRVPLPTELLVIVVATLVSHFGQLHKRFGSSVAGDIPTGFMPPQVPEPRLMQRVALDAVALALVAAAFSISLAEMFARSHGYSVRANQELLAVHRGHLRGACQGVGLPGCGGSPADALVWAGTGTCMLVSTEAGLLAGVILSLLSLAGRTQKPRTALLARIGDTAFYEDATEFEGLVPEPGVRVFRFGGPLYYANKDFFLQSLYSLTGLDAGCMAARRKEGGSETGVGEGGPAQGEDLGPVSTRAALVPAAAGFHTVVIDCAPLLFLDAAGVSTLQDLRRDYGALGISLLLACCSPPVRDILSRGGFLGEGPGDTAEEEQLFLSVHDAVQTARARHRELEATDAHL

SEQ ID NO: 15 - DNA, 406 bp, Homo sapiens
gtggaggaggctttctgtaatacctggaagctgaccgaccagaactttgatgagtacatgaaggctctagggatgggctttgtcactaggcaggtgggaaatgtggacaaaccaagagtgattatcagtcaagaagaagacaaggtggtgatcaggattcaaagtatgttcaagaacacagaggttagtttccatctgggagaagagtttgatgaaaccactacagatgacagaaactgcaagtttgttgttagtctggacagagacaaactcattcacatacagaaatgggatgacaaagaaacatattttataagagaaattaagtatggtgaaatggttatgacctttacttttggtgatgatgtggttgccgttcaccactataagaaggcataaaaatgtt

SEQ ID NO: 16 - protein, 132 aa, Homo sapiens
VEEAFCNTWKLTDQNFDEYMKALGMGFVTRQVGNVDKPRVIISQEEDKVVIRIQSMFKNTEVSFHLGEEFDETTTDDRNCKFVVSLDRDKLIHIQKWDDKETYFIREIKYGEMVMTFTFGDDVVAVHHYKKA

SEQ ID NO: 17 - DNA, 418 bp, Homo sapiens
ataatggtaagggtggaggaggctttctgtaatacctggaagctgaccgaccagaactttgatgagtacatgaaggctctagggatgggctttgtcactaggcaggtgggaaatgtggacaaaccaagagtgattatcagtcaagaagaagacaaggtggtgatcaggattcaaagtatgttcaagaacacagaggttagtttccatctgggagaagagtttgatgaaaccactacagatgacagaaactgcaagtttgttgttagtctggacagagacaaactcattcacatacagaaatgggatgacaaagaaacatattttataagagaaattaagtatggtgaaatggttatgacctttacttttggtgatgatgtggttgccgttcaccactataagaaggcataaaaatgtt

SEQ ID NO: 18 - protein, 135 aa, Homo sapiens
MVRVEEAFCNTWKLTDQNFDEYMKALGMGFVTRQVGNVDKPRVIISQEEDKVVIRIQSMFKNTEVSFHLGEEFDETTTDDRNCKFVVSLDRDKLIHIQKWDDKETYFIREIKYGEMVMTFTFGDDVVAVHHYKKA

SEQ ID NO: 19 - DNA, 1119 bp, Homo sapiens
atggagcacacgcacgcccacctcgcagccaacagctcgctgtcttggtggtcccccggctcggcctgcggcttgggtttcgtgcccgtggtctactacagcctcttgctgtgcctcggtttaccagcaaatatcttgacagtgatcatcctctcccagctggtggcaagaagacagaagtcctcctacaactatctcttggcactcgctgctgccgacatcttggtcctctttttcatagtgtttgtggacttcctgttggaagatttcatcttgaacatgcagatgcctcaggtccccgacaagatcatagaagtgctggaattctcatccatccacacctccatatggattactgtaccgttaaccattgacaggtatatcgctgtctgccacccgctcaagtaccacacggtctcatacccagcccgcacccggaaagtcattgtaagtgtttacatcacctgcttcctgaccagcatcccctattactggtggcccaacatctggactgaagactacatcagcacctctgtgcatcacgtcctcatctggatccactgcttcaccgtctacctggtgccctgctccatcttcttcatcttgaactcaatcattgtgtacaagctcaggaggaagagcaattttcgtctccgtggctactccacggggaagaccaccgccatcttgttcaccattacctccatctttgccacactttgggccccccgcatcatcatgattctttaccacctctatggggcgcccatccagaaccgctggctggtacacatcatgtccgacattgccaacatgctagcccttctgaacacagccatcaacttcttcctctactgcttcatcagcaagcggttccgcaccatggcagccgccacgctcaaggctttcttcaagtgccagaagcaacctgtacagttctacaccaatcataacttttccataacaagtagcccctggatctcgccggcaaactcacactgcatcaagatgctggtgtaccagtatgacaaaaatggaaaacctataaaaagtcgtaatgacagcaaaagctcctaccagtttgaagatgccattggagcttgtgtcatcatcctgtga

SEQ ID NO: 20 - protein, 372 aa, Homo sapiens
MEHTHAHLAANSSLSWWSPGSACGLGFVPVVYYSLLLCLGLPANILTVIILSQLVARRQKSSYNYLLALAAADILVLFFIVFVDFLLEDFILNMQMPQVPDKIIEVLEFSSIHTSIWITVPLTIDRYIAVCHPLKYHTVSYPARTRKVIVSVYITCFLTSIPYYWWPNIWTEDYISTSVHHVLIWIHCFTVYLVPCSIFFILNSIIVYKLRRKSNFRLRGYSTGKTTAILFTITSIFATLWAPRIIMILYHLYGAPIQNRWLVHIMSDIANMLALLNTAINFFLYCFISKRFRTMAAATLKAFFKCQKQPVQFYTNHNFSITSSPWISPANSHCIKMLVYQYDKNGKPIKSRNDSKSSYQFEDAIGACVIIL

SEQ ID NO: 21 - DNA, 1343 bp, Homo sapiens
tatggagcacacgcacgcccacctcgcagccaacagctcgctgtcttggtggtcccccggctcggcctgcggcttgggtttcgtgcccgtggtctactacagcctcttgctgtgcctcggtttaccagcaaatatcttgacagtgatcatcctctcccagctggtggcaagaagacagaagtcctcctacaactatctcttggcactcgctgctgccgacatcttggtcctctttttcatagtgtttgtggacttcctgttggaagatttcatcttgaacatgcagatgcctcaggtccccgacaagatcatagaagtgctggaattctcatccatccacacctccatatggattactgtaccgttaaccattgacaggtatatcactgtctgccacccgctcaagtaccacacggtctcatacccagcccgcacccggaaagtcattgtaagtgtttacatcacctgcttcctgaccagcatcccctattactggtggcccaacatctggactgaagactacatcagcacctctgtgcatcacgtcctcatctggatccactgcttcaccgtctacctggtgccctgctccatcttcttcatcttgaactcaatcattgtgtacaagctcaggaggaagagcaattttcgtctccgtggctactccacggggaagaccaccgccatcttgttcaccattacctccatctttgccacactttgggccccccgcatcatcatgattctttaccacctctatggggcgcccatccagaaccgctggctggtacacatcatgtccgacattgccaacatgctagcccttctgaacacagccatcaacttcttcctctactgcttcatcagcaagcggttccgcaccatggcagccgccacgctcaaggctttcttcaagtgccagaagcaacctgtacagttctacaccaatcataacttttccataacaagtagcccctggatctcgccggcaaactcacactgcatcaagatgctggtgtaccagtatgacaaaaatggaaaacctataaaagtatccccgtgattccataggtgtggcaactactgcctctgtctaatccatttccagatgggaaggtgtcccatcctatggctgagcagctctccttaagagtgctaatccgatttcctgtctcccgcagactgggcaattctcagactggtagatgagaagagatggaagagaagaaaggagagcatgaagcttgtttttacttatgcatttatttccacagagtcgtaatgacagcaaaagctcctaccagtttgaagatgccattggagcttgtgtcatcatcctgtga

SEQ ID NO: 22 - protein, 353 aa, Homo sapiens
MEHTHAHLAANSSLSWWSPGSACGLGFVPVVYYSLLLCLGLPANILTVIILSQLVARRQKSSYNYLLALAAADILVLFFIVFVDFLLEDFILNMQMPQVPDKIIEVLEFSSIHTSIWITVPLTIDRYITVCHPLKYHTVSYPARTRKVIVSVYITCFLTSIPYYWWPNIWTEDYISTSVHHVLIWIHCFTVYLVPCSIFFILNSIIVYKLRRKSNFRLRGYSTGKTTAILFTITSIFATLWAPRIIMILYHLYGAPIQNRWLVHIMSDIANMLALLNTAINFFLYCFISKRFRTMAAATLKAFFKCQKQPVQFYTNHNFSITSSPWISPANSHCIKMLVYQYDKNGKPIKVSP

SEQ ID NO: 23 - DNA, 2392 bp, Homo sapiens
tcggcgcgaggattcagtggatgaagagtacttattgctagaatgttcttcctcatatgaacttgacaacgttctgctctctaattccatttatttagctgtttcgaattgatgaggatgcagcgaggagctgccatctgtgaaatgggccctcaccagactccgaatctgccagtatcttgctcttgggacttccagcctccggaactgtaaacacagcaacaaaaaagttatgagaaccaagagctctgagaaggctgccaacgatgatcacagtgtccgtgtggcccgtgaagatgtcagagagagttgcccacctcttggtctggaaaccttaaaaatcacagacttccagctccatgcctccacggtgaagcgctatggcctgggggcacatcgagggagactcaacatccaggcgggcattaatgaaaatgatttttatgacggagcgtggtgcgcgggaagaaatgacctccagcagtggattgaagtggatgctcggcgcctgaccagattcactggtgtcatcactcaagggaggaactccctctggctgagtgactgggtgacatcctataaggtcatggtgagcaatgacagccacacgtgggtcactgttaagaatggatctggagacatgatatttgagggaaacagtgagaaggagatccctgttctcaatgagctacccgtccccatggtggcccgctacatccgcataaaccctcagtcctggtttgataatgggagcatctgcatgagaatggagatcctgggctgcccactgccagatcctaataattattatcaccgccggaacgagatgaccaccactgatgacctggattttaagcaccacaattataaggaaatgcgccaggtacagttgatgaaagttgtgaatgaaatgtgtcccaatatcaccagaatttacaacattggaaaaagccaccagggcctgaagctgtatgctgtggagatctcagatcaccctggggagcatgaagtcggtgagcccgagttccactacatcgcgggggcccacggcaatgaggtgctgggccgggagctgctgctgctgctggtgcagttcgtgtgtcaggagtacttggcccggaatgcgcgcatcgtccacctggtggaggagacgcggattcacgtcctcccctccctcaaccccgatggctacgagaaggcctacgaagggggctcggagctgggaggctggtccctgggacgctggacccacgatggaattgacatcaacaacaactttcctgatttaaacacgctgctctgggaggcagaggatcgacagaatgtccccaggaaagttcccaatcactatattgcaatccctgagtggtttctgtcggaaaatgccacggtggtggctgccgagaccagagcagtcatagcctggatggaaaaaatcccttttgtgctgggcggcaacctgcagggcggcgagctggtggtggcgtacccctacgacctggtgcggtccccctggaagacgcaggaacacacccccacccccgacgaccacgtgttccgctggctggcctactcctatgcctccacacaccgcctcatgacagacgcccggaggagggtgtgccacacggaggacttccaaaaggaggagggcactgtcaatggggcctcctggcacaccgtcgctggaagtctgaacgatttcagctaccttcatacaaactgcttcgaactgtccatctacgtgggctgtgataaatacccacatgagagccagctgcccgaggagtgggagaataaccgggaatctctgatcgtgttcatggagcaggttcatcgtggcattaaaggcttggtgagagattcacatggaaaaggaatcccaaacgccattatctccgtagaaggcattaaccatgacatccgaacagccaacgatggggattactggcgcctcctgaaccctggagagtatgtggtcacagcaaaggccgaaggtttcactgcatccaccaagaactgtatggttggctatgacatgggggccacaaggtgtgacttcacacttagcaaaaccaacatggccaggatccgagagatcatggagaagtttgggaagcagcccgtcagcctgccagccaggcggctgaagctgcgggggcggaagagacgacagcgtgggtgaccctcctgggcccttgagactcgtctgggacccatgcaaattaaaccaacctggtagtagctccatagtggactcactcactgttgtttcctctgtaattcaagaagtgcctggaagagagggtgcattgtgaggcaggtcccaaaagggaaggctggaggctgaggctgttttcttttctttgttcccatttatccaaataacttg

SEQ ID NO: 24 - protein, 650 aa, Homo sapiens
MRTKSSEKAANDDHSVRVAREDVRESCPPLGLETLKITDFQLHASTVKRYGLGAHRGRLNIQAGINENDFYDGAWCAGRNDLQQWIEVDARRLTRFTGVITQGRNSLWLSDWVTSYKVMVSNDSHTWVTVKNGSGDMIFEGNSEKEIPVLNELPVPMVARYIRINPQSWFDNGSICMRMEILGCPLPDPNNYYHRRNEMTTTDDLDFKHHNYKEMRQVQLMKVVNEMCPNITRIYNIGKSHQGLKLYAVEISDHPGEHEVGEPEFHYIAGAHGNEVLGRELLLLLVQFVCQEYLARNARIVHLVEETRIHVLPSLNPDGYEKAYEGGSELGGWSLGRWTHDGIDINNNFPDLNTLLWEAEDRQNVPRKVPNHYIAIPEWFLSENATVVAAETRAVIAWMEKIPFVLGGNLQGGELVVAYPYDLVRSPWKTQEHTPTPDDHVFRWLAYSYASTHRLMTDARRRVCHTEDFQKEEGTVNGASWHTVAGSLNDFSYLHTNCFELSIYVGCDKYPHESQLPEEWENNRESLIVFMEQVHRGIKGLVRDSHGKGIPNAIISVEGINHDIRTANDGDYWRLLNPGEYVVTAKAEGFTASTKNCMVGYDMGATRCDFTLSKTNMARIREIMEKFGKQPVSLPARRLKLRGRKRRQRG

SEQ ID NO: 25 - DNA, 328 bp, Homo sapiens
aaataagattgaggaagctcctgaagaagtcttatgtcctcccaagtacttaaagctttcaccaaaacacccagaatcaaatactgctggaatggacatctttgccaaattctctgcatacatcaagaattcaaggccagaggttaatgaagcattagtgaagcatctcttaaaaaccctgcagaaaatggaatatctgaattctcctctccctgatgaaattgatgaaaatagcatgcaggacactaagttttctacacataaatttctgaatggcaataaaatggcattagctgattgccatctgctgcccaaactgcatattgtc

SEQ ID NO: 26 - DNA, 331 bp, Homo sapiens
aaataagattgaggaatttcttgaagaagtcttatgccctcccaagtacttaaagctttcaccaaaacacccagaatcaaatactgctggaatggacatctttgccaaattctctgcatatatcaagaattcaaggccagaggctaatgaagcactggagaggggtctcctgaaaaccctgcagaaactggatgaatatctgaattctcctctccctgatgaaattgatgaaaatagtatggaggacataaagttttctacacgtaaatttctggatggcaatgaaatgacattagctgattgcaacctgctgcccaaactgcatattgtc

SEQ ID NO: 27 - protein, 247 aa, Homo sapiens
MALSMPLNGLKEEDKEPLIELFVKAGSDGESIGNCPFSQRLFMILWLKGVVFSVTTVDLKRKPADLQNLAPGTHPPFITFNSEVKTDVNKIEEFLEEVLCPPKYLKLSPKHPESNTAGMDIFAKFSAYIKNSRPEANEALERGLLKTLQKLDEYLNSPLPDEIDENSMEDIKFSTRRFLDGDEMTLADCNLLPKLHIVKVVAKKYRNFDIPKGMTGIWRYLTNAYSRDEFTNTCPSDKEVEIAYSDV

SEQ ID NO: 28 - DNA, 550 bp, Homo sapiens
tctgaggacacagccacactcttgtcatgccattgcccttctattctttccttataacatcatgtaagagggcacagcatgtttcccatgctggaccctgctctgctcactccacacaccttctgacacccaccatggacactgttcagcaactggaagaaagagggcacctgatggacagcaaaggctttgatgaataataaatacatgaaggaactaggagtgggactagccctctgcgaaaaaaagggtgctatggccaaaaaagattgtattagcttttttgatggcaaaaacctcaccataaaaatggagagtactttaaaatcatacagttttctcacactcaggggagggaaattcaaagaaactacaggtgacggcagaaaaactcagactgcacctttacatatggcacattggttcgacatcagaagtggaatggaaaggaaggcaaaataagaaaattgaaagacaggaaattagtggtggactgcatcataaacaatgtcacctgtactcagatctatgaaaaagtagaataaaaact

SEQ ID NO: 29 - protein, 136 aa, Homo sapiens
MDTVQQLEERGHLMDSKGFDENKYMKELGVGLALCEKKGAMAKKDCISFFDGKNLTIKMESTLKSYSFLTLRGGKFKETTGDGRKTQTCTFTYGTLVRHQKWNGKEGKIRKLKDRKLVVDCIINNVTCTQIYEKVE

SEQ ID NO: 30 - protein, 135 aa, Homo sapiens
MATVQQLEGRWRLVDSKGFDEYMKELGVGIALRKMGAMAKPDCIITCDGKNLTIKTESTLKTTQFSCTLGEKFEETTADGRKTQTVCNFTDGALVQHQEWDGKESTITRKLKDGKLVVECVMNNVTCTRIYEKVE

SEQ ID NO: 31 - protein, 135 aa, Homo sapiens; identical to SEQ ID NO: 30

SEQ ID NO: 32 - DNA, 512 bp, Homo sapiens
atgctgccgccgccgcggcccgcagctgccttggcgctgcctgtgctcctgctactgctggtggtgctgacgccgcccccgaccggcgcaaggccatccccaggcccagattacctgcggcgcggctggatgcggctgctagcggagggcgagggctgcgctccctgccggccagaagagtgcgccgcgccgcggggctgcctggcgggcagggtgcgcgacgcgtgcggctgctgctgggaatgcgccaacctcgagggccagctctgcgacctggaccccagtgctcacttctacgggcactgcggcgagcagcttgagtgccggctggacacaggcggcgacctgagccgcggagaggtgccggaacctctgtgtgcctgtcgttcgcagagtccgctctgcgggtccgacggtcacacctactcccagatctgccgcctgcaggaggcggcccgcgctcggcccgatgccaacctcactgtggcacacccggggccctgcgaatcggg

SEQ ID NO: 33 - DNA, 512 bp, Homo sapiens; identical to SEQ ID NO: 32

SEQ ID NO: 34 - protein, 304 aa, Homo sapiens [sequence omitted]

SEQ ID NO: 35 - DNA, 1308 bp, Homo sapiens
cagcatgagcttcaccactcgctccaccttctccaccaactaccggtccctgggctctgtccaggcgcccagctacggcgcccggccggtcagcagcgcggccagcgtctatgcaggcgctgggggctctggttcccggatctccgtgtcccgctccaccagcttcaggggcggcatggggtccgggggcctggccaccgggatagccgggggtctggcaggaatgggaggcatccagaacgagaaggagaccatgcaaagcctgaacgaccgcctggcctcttacctggacagagtgaggagcctggagaccgagaaccggaggctggagagcaaaatccgggagcacttggagaagaagggaccccaggtcagagactggagccattacttcaagatcatcgaggacctgagggctcagatcttcgcaaatactgtggacaatgcccgcatcgttctgcagattgacaatgcccgtcttgctgctgatgactttagagtcaagtatgagacagagctggccatgcgccagtctgtggagaacgacatccatgggctccgcaaggtcattgatgacaccaatatcacacgactgcagctggagacagagatcgaggctctcaaggaggagctgctcttcatgaagaagaaccacgaagaggaagtaaaaggcctacaagcccagattgccagctctgggttgaccgtggaggtagatgcccccaaatctcaggacctcgccaagatcatggcagacatccgggcccaatatgacgagctggctcggaagaaccgagaggagctagacaagtactggtctcagcagattgaggagagcaccacagtggtcaccacacagtctgctgaggttggagctgctgagacgacgctcacagagctgagacgtacagtccagtccttggagatcgacctggactccatgagaaatctgaaggccagcttggagaacagcctgagggaggtggaggcccgctacgccctacagatggagcagctcaacgggatcctgctgcaccttgagtcagagctggcacagacccgggcagagggacagcgccaggcccaggagtatgaggccctgctgaacatcaaggtcaagctggaggctgagatcgccacctaccgccgcctgctggaagatggcgaggactttaatcttggtgatgccttggacagcagcaactccatgcaaaccatccaaaagaccaccacccgccggatagtggatggcaaagtggtgtctgagaccaatgacaccaaagttctgaggcattaagccagcagaag

SEQ ID NO: 36 - protein, 430 aa, Homo sapiens
MSFTTRSTFSTNYRSLGSVQAPSYGARPVSSAASVYAGAGGSGSRISVSRSTSFRGGMGSGGLATGIAGGLAGMGGIQNEKETMQSLNDRLASYLDRVRSLETENRRLESKIREHLEKKGPQVRDWSHYFKIIEDLRAQIFANTVDNARIVLQIDNARLAADDFRVKYETELAMRQSVENDIHGLRKVIDDTNITRLQLETEIEALKEELLFMKKNHEEEVKGLQAQIASSGLTVEVDAPKSQDLAKIMADIRAQYDELARKNREELDKYWSQQIEESTTVVTTQSAEVGAAETTLTELRRTVQSLEIDLDSMRNLKASLENSLREVEARYALQMEQLNGILLHLESELAQTRAEGQRQAQEYEALLNIKVKLEAEIATYRRLLEDGEDFNLGDALDSSNSMQTIQKTTTRRIVDGKVVSETNDTKVLRH

SEQ ID NO: 37 - protein, 722 aa, Mus musculus
MWGLLLAVTAFAPSVGLGLGAPSASVPGLAPGSTLAPHSSVAQPSTKANETSERHVRLRVIKKKKIVVKKRKKLRHPGPLGTARPVVPTHPAKTLTLPEKQEPGCPPLGLESLRVSDSQLEASSSQSFGLGAHRGRLNIQSGLEDGDLYDGAWCAEQQDTEPWLQVDAKNPVRFAGIVTQGRNSVWRYDWVTSFKVQFSNDSQTWWKSRNSTGMDIVFPANSDAETPVLNLLPEPQVARFIRLLPQTWFQGGVPCLRAEILACPVSDPNDLFPEAHTLGSSNSLDFRHHNYKAMRKLMKQVNEQCPNITRIYSIGKSHQGLKLYVMEMSDHPGEHELGEPEVRYVAGMHGNEALGRELLLLLMQFLCHEFLRGDPRVTRLLTETRIHLLPSMNPDGYETAYHRGSELVGWAEGRWTHQGIDLNHNFADLNTQLWYAEDDGLVPDTVPNHHLPLPTYYTLPNATVAPETWAVIKWMKRIPFVLSANLHGGELVVSYPFDMTRTPWAARELTPTPDDAVFRWLSTVYAGTNRAMQDTDRRPCHSQDFSLHGNVINGADWHTVPGSMNDFSYLHTNCFEVTVELSCDKFPHEKELPQEWENNKDALLTYLEQVRMGITGVVRDKDTELGIADAVIAVEGINHDVTTAWGGDYWRLLTPGDYVVTASAEGYHTVRQHCQVTFEEGPVPCNFLLTKTPKERLRELLATRGKLPPDLRRKLERLRGQK

SEQ ID NO: 38 - protein, 734 aa, Homo sapiens
MWGLLLALAAFAPAVGPALGAPRNSVLGLAQPGTTKVPGSTPALHSSPAQPPAETANGTSEQHVRIRVIKKKKVIMKKRKKLTLTRPTPLVTAGPLVTPTPAGTLDPAEKQETGCPPLGLESLRVSDSRLEASSSQSFGLGPHRGRLNIQSGLEDGDLYDGAWCAEEQDADPWFQVDAGHPTRFSGVITQGRNSVWRYDWVTSYKVQFSNDSRTWWGSRNHSSGMDAVFPANSDPETPVLNLLPEPQVARFIRLLPQTWLQGGAPCLRAEILACPVSDPNDLFLEAPASGSSDPLDFQHHNYKAMRKLMKQVQEQCPNITRIYSIGKSYQGLKLYVMEMSDKPGEHELGEPEVRYVAGMHGNEALGRELLLLLMQFLCHEFLRGNPQVTRLLSEMRIHLLPSMNPDGYEIAYHRGSELVGWAEGRWNNQSIDLNHNFADLNTPLWEAQDDGKVPHIVPNHHLPLPTYYTLPNATVAPETRAVIKWMKRIPFVLSANLHGGELVVSYPFDMTRTPWAARELTPTPDDAVFRWLSTVYAGSNLAMQDTSRRPCHSQDFSVHGNIINGADWHTVPGSMNDFSYLHTNCFEVTVELSCDKFPHENELPQEWENNKDALLTYLEQVRMGIAGVVRDKDTELGIADAVIAVDGINHDVTTAWGGDYWRLLTPGDYMVTASAEGYHSVTRNCRVTFEEGPFPCNFVLTKTPKQRLRELLAAGAKVPPDLRRRLERLRGQKD

SEQ ID NO: 39 - DNA, 267 bp, Homo sapiens
ggaaggacaccgacccgtccatctaccggatccacgctggggacgtgtatctctacgggggccgggggctgctgaacgtcagccggatcatcgtccaccccaactatgtcactgcggggctgggtgcggatgtggccctgctccagctggtgagccccatgatcggagccgctaatgtcaggacggtcaagctctccccggtctcgctggagctcaccccgaaggaccagtgctgggtgactggctggggagcgatcaggatgttcg

SEQ ID NO: 40 - DNA, 267 bp, Homo sapiens
ggaaggacaccgacccgtccatctaccggatccacgctggggacgtgtatctctacgggggccgggggctgctgaacgtcagccggatcatcgtccaccccaactatgtcactgcggggctgggtgcggatgtggccctgctccagctggtgagccccatgatctgagccgctaatgtcaggacggtcaagctctccccggtctcgctggagctcaccccgaaggaccagtgctgggtgactggctggggagcgatcaggatgttcg

SEQ ID NO: 41 - protein, 255 aa, Homo sapiens
PVPENDLVGIVGGHNAPPGKWPWQVSLRVYSYHWASWAHICGGSLIHPQWVLTAAHCIFWKDTDPSIYRIHAGDVYLYGGRGLLNVSRIIVHPNYVTAGLGADVALLQLVSPMIGAANVRTVKLSPVSLELTPKDQCWVTGWGAIRMFESLPPPYRLQQASVQVLENAVCEQPYRNASGHTGDRQLILDDMLCAGSEGRDSCQGDSGGPLVCRLRGSWRLVGVVSWGYGCTLRDFPGVYTHVQIYVLWILQQVGE

SEQ ID NO: 42 - protein, 252 aa, Mus musculus
PRPANQRVGIVGGHEASESKWPWQVSLRFKLNYWIHFCGGSLIHPQWVLTAAHCVGPHIKSPQLFRVQLREQYLYYGDQLLSLNRIVVHPHYYTAEGGADVALLELEVPVNVSTHIHPISLPPASETFPPGTSCWVTGWGDIDNDEPLPPPYPLKQVKVPIVENSLCDRKYHTGLYTGDDFPIVHDGMLCAGNTRRDSCQGDSGGPLVCKVKGTWLQAGVVSWGEGCAQPNKPGIYTRVTYYLDWIHRYVPE

SEQ ID NO: 43 - protein, 278 aa, Homo sapiens; identical to SEQ ID NO: 12

SEQ ID NO: 44 - protein, 275 aa, Homo sapiens
MLNLLLLALPVLASRAYAAPAPGQALQRVGIVGGQEAPRSKWPWQVSLRVHGPYWMHFCGGSLIHPQWVLTAAHCVGPDVKDLAALRVQLREQHLYYQDQLLPVSRIIVHPQFYTAQIGADIALLELEEPVKVSSHVHTVTLPPASETFPPGMPCWVTGWGDVDNDERLPPPFPLKQVKVPIMENHICDAKYHLGAYTGDDVRIVRDDMLCAGNTRRDSCQGDSGGPLVCKVNGTWLQAGVVSWGEGCAQPNRPGIYTRVTYYLDWIHHYVPKKP

SEQ ID NO: 45 - DNA, 1170 bp, Homo sapiens
caggtcggccacgggacctgacgcaacaggatggacgagtcccctgagcctctgcagcagggcagagggccggtgccggtccgacgccagcgcccagcaccccggggtctgcgtgagatgctgaaggccaggctgtggtgcagctgctcgtgcagtgtgctgtgcgtccgggcgctggtgcaggacctgctccccgccacgcgctggctgcgtcagtaccgcccgcgggagtacctggcaggcgacgtcatgtctgggctggtcatcggcatcatcctggtcccgcaggccatcgcctactcattgctggccgggctgcagcccatctacagcctctatacgtccttcttcgccaacctcatctacttcctcatgggcacctcacggcatgtctccgtgggcatcttcagcctgctttgcctcatggtggggcaggtggtggaccgggagctccagctggccggctttgacccctcccaggacggcctgcagcccggagccaacagcagcaccctcaacggctcggctgccatgctggactgcgggcgtgactgctacgccatccgtgtcgccaccgccctcacgctgatgaccgggctttaccaggtcctcatgggcgtcctccggctgggcttcgtgtccgcctacctctcacagccactgctcgatggctttgccatgggggcctccgtgaccatcctgacctcgcagctcaaacacctgctgggcgtgcggatcccgcggcaccaggggcccggcatggtggtcctcacatggctgagcctgctgcgcggcgccgggcaggccaacgtgtgcgacgtggtcaccagcacggtgtgcctggcggtgctgctagccgcgaaggagctctcagaccgctaccgacaccgcctgagggtgccgctgcccacggagctgctggtcatcgtggtggccacactcgtgtcgcacttcgggcagctccacaagcgctttggctcgagcgtggctggcgacatccccacgggtttcatgccccctcaggtcccagagcccaggctgatgcagcgtgtggctttggatgccgtggccctggccctcgtggctgccgccttctccatctcgctggcggagatgttcgcccgcagtcacggctactctgtgcgtgccaaccaggagctgctggctgtg

SEQ ID NO: 46 - DNA, 1170 bp, Homo sapiens
caggtcggccacgggacctgacgcaacaggatggacgagtcccctgagcctctgcagcagggcagagggccggtgccggtccgacggcagcgcccagcaccccggggtctgcgtgagatgctgaaggccaggctgtggtgcagctgctcgtgcagtgtgctgtgcgtccgggcgctggtgcaggacctgctccccgccacgcgctggctgcgtcagtaccgcccgcgggagtacctggcaggcgacgtcatgtctgggctggtcatcggcatcatcctggtgccgcaggccatcgcctactcattgctggccgggctgcagcccatctacagcctctatacgtccttcttcgccaacctcatctacttcctcatgggcacctcacggcatgtctccgtgggcatcttcagcctgctttgcctcatggtggggcaggtggtggaccgggagctccagctggccggctttgacccctcccaggacggcctgcagcccggagccaacagcagcaccctcaacggctcggctgccatgctggactgcgggcgtgactgctacgccatccgtgtcgccaccgccctcacgctgatgaccgggctttaccaggtcctcatgggcgtcctccggctgggcttcgtgtccgcctacctctcacagccactgctcgatggctttgccatgggggcctccgtgaccatcctgacctcgcagctcaaacacctgctgggcgtgcggatcccgcggcaccaggggcccggcatggtggtcctcacatggctgagcctgctgcgcggcgccgggcaggccaacgtgtgcgacgtggtcaccagcacggtgtgcctggcggtgctgctagccgcgaaggagctctcagaccgctaccgacaccgcctgagggtgccgctgcccacggagctgctggtcatcgtggtggccacactcgtgtcgcacttcgggcagctccacaagcgctttggctcgagcgtggctggcgacatccccacgggtttcatgccccctcaggtcccagagcccaggctgatgcagcgtgtggctttggatgccgtggccctggccctcgtggctgccgccttctccatctcgctggcggagatgttcgcccgcagtcacggctactctgtgcgtgccaaccaggagctgctggctgtg

SEQ ID NO: 47 - protein, 434 aa, Homo sapiens
MDESPEPLQQGRGPVPVRRQRPAPRGLREMLKARLWCSCSCSVLCVRALVQDLLPATRWLRQYRPREYLAGDVMSGLVIGIILVPQAIAYSLLAGLQPIYSLYTSFFANLIYFLMGTSRHVSVGIFSLLCLMVGQVVDRELQLAGFDPSQDGLQPGANSSTLNGSAAMLDCGRDCYAIRVATALTLMTGLYQVLMGVLRLGFVSAYLSQPLLDGFAMGASVTILTSQLKHLLGVRIPRHQGPGMVVLTWLSLLRGAGQANVCDVVTSTVCLAVLLAAKELSDRYRHRLRVPLPTELLVIVVATLVSHFGQLHKRFGSSVAGDIPTGFMPPQVPEPRLMQRVALDAVALALVAAAFSISLAEMFARSHGYSVRANQELLAVHRGHLRGACQGVGLPGCGGSPADALVWAGTGTCMLVSTEAGLLAGVILSLLSLA

SEQ ID NO: 48 - protein, 435 aa, Rattus rattus
MDASPEPPQKGGTLVLVRRQPPVSQGLLETLKARLKKSCTCSMPCAQALVQGLFPVIRWLPQYRLKEYLAGDVMSGLVIGIILVPQAIAYSLLAGLQPIYSLYTSFFANLIYFLMGTSRHVNVGIFSLLCLMVGQVVDRELQLAGFDPSQDSLGPGNNDSTLNNTATLTVGLQDCGRDCHAIRIATALTLMAGLYQVLMGILRLGFVSTYLSQPLLDGFAMGASVTILTSQAKHLLGVRIPRHQGLGMVIHTWLSLLQNVGQANLCDVVTSAVCLAVLLTAKELSDRYRHYLKVPVPTELLVIVVATIASHFGQLHTRFGSSVAGNIPTGFVAPQIPDPKIMWSVALDAMSLALVGSAFSISLAEMFARSHGYSVSANQELLAVGCCNVLPAFFHCFATSAALSKTLVKIATGCQTQLSSVVSAAVVLLVLLVLA

SEQ ID NO: 49 - DNA, 404 bp, Homo sapiens
tggaggaggctttctgtaatacctggaagctgaccgaccagaactttgatgagtacatgaaggctctagggatgggctttgtcactaggcaggtgggaaatgtggacaaaccaagagtgattatcagtcaagaagaagacaaggtggtgatcaggattcaaagtatgttcaagaacacagaggttagtttccatctgggagaagagtttgatgaaaccactacagatgacagaaactgcaagtttgttgttagtctggacagagacaaactcattcacatacagaaatgggatgacaaagaaacatattttataagagaaattaagtatggtgaaatggttatgacctttacttttggtgatgatgtggttgccgttcaccactataagaaggcataaaaatgt

SEQ ID NO: 50 - DNA, 404 bp, Homo sapiens
tggtggaggctttctgtgctacctggaagctgaccaacagtcagaactttgatgagtacatgaaggctctaggcgtgggctttgccactaggcaggtgggaaatgtgaccaaaccaacggtaattatcagtcaagaaggagacaaagtggtcatcaggactctcagcacattcaagaacacggagattagtttccagctgggagaagagtttgatgaaaccactgcagatgatagaaactgtaagtctgttgttagcctggatggagacaaacttgttcacatacagaaatgggatggcaaagaaacaaattttgtaagagaaattaaggatggcaaaatggttatgacccttacttttggtgatgtggttgctgttcgccactatgagaaggcataaaaatgt

SEQ ID NO: 51 - protein, 130 aa, Homo sapiens
EAFCNTWKLTDQNFDEYMKALGMGFVTRQVGNVDKPRVIISQEEDKVVIRIQSMFKNTEVSFHLGEEFDETTTDDRNCKFVVSLDRDKLIHIQKWDDKETYFIREIKYGEMVMTFTFGDDVVAVHHYKKA

SEQ ID NO: 52 - protein, 130 aa, Homo sapiens
EAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKA

SEQ ID NO: 53 - protein, 130 aa, Homo sapiens; identical to SEQ ID NO: 51

SEQ ID NO: 54 - protein, 130 aa, Homo sapiens; identical to SEQ ID NO: 52

SEQ ID NO: 55 - protein, 132 aa, Homo sapiens; identical to SEQ ID NO: 16

SEQ ID NO: 56 - protein, 132 aa, Homo sapiens; identical to SEQ ID NO: 16

SEQ ID NO: 57 - protein, 272 aa, Homo sapiens
ACGLGFVPVVYYSLLLCLGLPANILTVIILSQLVARRQKSSYNYLLALAAADILVLFFIVFVDFLLEDFILNMQMPQVPDKIIEVLEFSSIHTSIWITVPLTIDRYIAVCHPLKYHTVSYPARTRKVIVSVYITCFLTSIPYYWWPNIWTEDYISTSVHHVLIWIHCFTVYLVPCSIFFILNSIIVYKLRRKSNFRLRGYSTGKTTAILFTITSIFATLWAPRIIMILYHLYGAPIQNRWLVHIMSDIANMLALLNTAINFFLYCFISKRFR

SEQ ID NO: 58 - protein, 272 aa, Homo sapiens; identical to SEQ ID NO: 57

SEQ ID NO: 59 - protein, 350 aa, Homo sapiens
MEHTHAHLAANSSLSWWSPGSACGLGFVPVVYYSLLLCLGLPANILTVIILSQLVARRQKSSYNYLLALAAADILVLFFIVFVDFLLEDFILNMQMPQVPDKIIEVLEFSSIHTSIWITVPLTIDRYITVCHPLKYHTVSYPARTRKVIVSVYITCFLTSIPYYWWPNIWTEDYISTSVHHVLIWIHCFTVYLVPCSIFFILNSIIVYKLRRKSNFRLRGYSTGKTTAILFTITSIFATLWAPRIIMILYHLYGAPIQNRWLVHIMSDIANMLALLNTAINFFLYCFISKRFRTMAAATLKAFFKCQKQPVQFYTNHNFSITSSPWISPANSHCIKMLVYQYDKNGKPIK

SEQ ID NO: 60 - protein, 350 aa, Homo sapiens
MEHTHAHLAANSSLSWWSPGSACGLGFVPVVYYSLLLCLGLPANILTVIILSQLVARRQKSSYNYLLALAAADILVLFFIVFVDFLLEDFILNMQMPQVPDKIIEVLEFSSIHTSIWITVPLTIDRYIAVCHPLKYHTVSYPARTRKVIVSVYITCFLTSIPYYWWPNIWTEDYISTSVHHVLIWIHCFTVYLVPCSIFFILNSIIVYKLRRKSNFRLRGYSTGKTTAILFTITSIFATLWAPRIIMILYHLYGAPIQNRWLVHIMSDIANMLALLNTAINFFLYCFISKRFRTMAAATLKAFFKCQKQPVQFYTNHNFSITSSPWISPANSHCIKMLVYQYDKNGKPIK

SEQ ID NO: 61 - protein, 657 aa, Homo sapiens [sequence omitted]

SEQ ID NO: 62 - protein, 654 aa, Homo sapiens
KHSNKKVMRTKSSEKAANDDHSVRVAREDVRESCPPLGLETLKITDFQLHASTVKRYGLGAHRGRLNIQAGINENDFYDGAWCAGRNDLQQWIEVDARRLTRFTGVITQGRNSLWLSDWVTSYKVMVSNDSHTWVTVKNGSGDMIFEGNSEKEIPVLNELPVPMVARYIRINPQSWFDNGSICMRMEILGCPLPDPNNYYHRRNEMTTTDDLDFKHHNYKEMRQLMKVVNEMCPNITRIYNIGKSHQGLKLYAVEISDHPGEHEVGEPEFHYIAGAHGNEVLGRELLLLLVQFVCQEYLARNARIVHLVEETRIHVLPSLNPDGYEKAYEGGSELGGWSLGRWTHDGIDINNNFPDLNTLLWEAEDRQNVPRKVPNHYIAIPEWFLSENATVAAETRAVIAWMEKIPFVLGGNLQGGELVVAYPYDLVRSPWKTQEHTPTPDDHVFRWLAYSYASTHRLMTDARRRVCHTEDFQKEEGTVNGASWHTVAGSLNDFSYLHTNCFELSIYVGCDKYPHESQLPEEWENNRESLIVFMEQVHRGIKGLVRDSHGKGIPNAIISVEGINHDIRTANDGDYWRLLNPGEYVVTAKAEGFTASTKNCMVGYDMGATRCDFTLSKTNMARIREIMEKFGKQPVSLPARRLKLRGRKRRQRG

SEQ ID NO: 63 - protein, 509 aa, Homo sapiens
NSEKEIPVLNELPVPMVARYIRINPQSWFDNGSICMRMEILGCPLPDPNNYYHRRNEMTTTDDLDFKHHNYKEMRQVQLMKVVNEMCPNITRIYNIGKSHQGLKLYAVEISDHPGEHEVGEPEFHYIAGAHGNEVLGRELLLLLVQFVCQEYLARNARIVHLVEETRIHVLPSLNPDGYEKAYEGGSELGGWSLGRWTHDGIDINNNFPDLNTLLWEAEDRQNVPRKVPNHYIAIPEWFLSENATVVAAETRAVIAWMEKIPFVLGGNLQGGELVVAYPYDLVRSPWKTQEHTPTPDDHVFRWLAYSYASTHRLMTDARRRVCHTEDFQKEEGTVNGASWHTVAGSLNDFSYLHTNCFELSIYVGCDKYPHESQLPEEWENNRESLIVFMEQVHRGIKGLVRDSHGKGIPNAIISVEGINHDIRTANDGDYWRLLNPGEYVVTAKAEGFTASTKNCMVGYDMGATRCDFTLSKTNMARIREIMEKFGKQPVSLPARRLKLRGRKRRQRG

SEQ ID NO: 64 - protein, 506 aa, Homo sapiens
NSEKEIPVLNELPVPMVARYIRINPQSWFDNGSICMRMEILGCPLPDPNNYYHRRNEMTTTDDLDFKHHNYKEMRQLMKVVNEMCPNITRIYNIGKSHQGLKLYAVEISDHPGEHEVGEPEFHYIAGAHGNEVLGRELLLLLLHFLCQEYSAQNARIVRLVEETRIHILPSLNPDGYEKAYEGGSELGGWSLGRWTHDGIDINNNFPDLNSLLWEAEDQQNAPRKVPNHYIAIPEWFLSENATVATETRAVIAWMEKIPFVLGGNLQGGELVVAYPYDMVRSLWKTQEHTPTPDDHVFRWLAYSYASTHRLMTDARRRVCHTEDFQKEEGTVNGASWHTVAGSLNDFSYLHTNCFELSIYVGCDKYPHESELPEEWENNRESLIVFMEQVHRGIKGIVRDLQGKGISNAVISVEGVNHDIRTASDGDYWRLLNPGEYVVTAKAEGFITSTKNCMVGYDMGATRCDFTLTKTNLARIREIMETFGKQPVSLPSRRLKLRGRKRRQRG

SEQ ID NO: 65 - DNA, 24 bp, Artificial Sequence
  Description of Artificial Sequencechemically synthesized oligonucleotide
tcacaggatgatgacacaagctcc

SEQ ID NO: 66 - DNA, 22 bp, Artificial Sequence
  Description of Artificial Sequencechemically synthesized oligonucleotide
atgtgatctttggctgtgaagt

SEQ ID NO: 67 - DNA, 23 bp, Artificial Sequence
  Description of Artificial Sequencechemically synthesized oligonucleotide
ctaccccatggcctccatcgagt

SEQ ID NO: 68 - DNA, 19 bp, Artificial Sequence
  Description of Artificial Sequencechemically synthesized oligonucleotide
ggatgtccaagccatcctt

SEQ ID NO: 69 - DNA, 18 bp, Artificial Sequence
  Description of Artificial Sequencechemically synthesized oligonucleotide
tgactgctgcccactgca

SEQ ID NO: 70 - DNA, 24 bp, Artificial Sequence
  Description of Artificial Sequencechemically synthesized oligonucleotide
caccgacccgtccatctaccggat

SEQ ID NO: 71 - DNA, 20 bp, Artificial Sequence
  Description of Artificial Sequencechemically synthesized oligonucleotide
gagatacacgtccccagcgt

SEQ ID NO: 72 - DNA, 22 bp, Artificial Sequence
  Description of Artificial Sequencechemically synthesized oligonucleotide
ctcaagtaccacacggtctcat

SEQ ID NO: 73 - DNA, 25 bp, Artificial Sequence
  Description of Artificial Sequencechemically synthesized oligonucleotide
ccgcacccggaaagtcattgtaagt

SEQ ID NO: 74 - DNA, 22 bp, Artificial Sequence
  Description of Artificial Sequencechemically synthesized oligonucleotide
tcaggaagcaggtgatgtaaac

SEQ ID NO: 75 - DNA, 20 bp, Artificial Sequence
  Description of Artificial Sequencechemically synthesized oligonucleotide
ggaagctgaccgaccagaac

SEQ ID NO: 76 - DNA, 29 bp, Artificial Sequence
  Description of Artificial Sequencechemically synthesized oligonucleotide
agcccatccctagagccttcatgtactca

SEQ ID NO: 77 - DNA, 22 bp, Artificial Sequence
  Description of Artificial Sequencechemically synthesized oligonucleotide
atttcccacctgcctagtgaca

SEQ ID NO: 78 - DNA, 21 bp, Artificial Sequence
  Description of Artificial Sequencechemically synthesized oligonucleotide
cagctcgctgtcttggtggtc

(19) United States
(12) Patent Application Publication
(10) Pub. No.: US 2004/0121380 A1
(43) Pub. Date: Jun. 24, 2004

(54) Novel polypeptides and nucleic acids encoding same

(76) Inventors: Raymond J. Taupier JR., East Haven, CT (US); Kumud Majumder, Stamford, CT (US); Steven K. Spaderna, Berlin, CT (US); Glennda Smithson, Guilford, CT (US); Peter S. Mezes, Old Lyme, CT (US); Corine A.M. Vernet, North Branford, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/689,832

(22) Filed: Oct. 20, 2003

Related U.S. Application Data
(62) Division of application No. 09/813,432, filed on Mar. 20, 2001, now abandoned.
(60) Provisional application No. 60/190,835, filed on Mar. 20, 2000.
(60) Provisional application No. 60/190,768, filed on Mar. 20, 2000.
(60) Provisional application No. 60/190,972, filed on Mar. 22, 2000.
(60) Provisional application No. 60/191,199, filed on Mar. 22, 2000.
(60) Provisional application No. 60/191,947, filed on Mar. 24, 2000.
(60) Provisional application No. 60/192,665, filed on Mar. 28, 2000.
(60) Provisional application No. 60/192,657, filed on Mar. 28, 2000.
(60) Provisional application No. 60/192,984, filed on Mar. 28, 2000.
(60) Provisional application No. 60/192,664, filed on Mar. 28, 2000.
(60) Provisional application No. 60/192,836, filed on Mar. 29, 2000.
(60) Provisional application No. 60/193,843, filed on Mar. 31, 2000.

(51) Int. Cl. C12Q 1/68; C07H 21/04; C07K 14/705; C07K 16/28

(52) U.S. Cl. 435/6; 435/69.1; 435/320.1; 435/325; 530/350; 530/388.1; 536/23.2

(57) ABSTRACT

The present invention provides novel isolated NOVX polynucleotides and polypeptides encoded by the NOVX polynucleotides. Also provided are the antibodies that immunospecifically bind to a NOVX polypeptide or any derivative, variant, mutant or fragment of the NOVX polypeptide, polynucleotide or antibody. The invention additionally provides methods in which the NOVX polypeptide, polynucleotide and antibody are utilized in the detection and treatment of a broad range of pathological states, as well as to other uses.

### RELATED APPLICATIONS

This application is a divisional application of U.S. Ser. No. 09/813,432, filed Mar. 20, 2001, which claims priority to U.S. Ser. No. 60/190,835, filed Mar. 20, 2000 (15966-729); U.S. Ser. No. 60/190,768, filed Mar. 20, 2000 (15966-734); U.S. Ser. No. 60/190,972, filed Mar. 22, 2000 (15966-735); U.S. Ser. No. 60/191,199, filed Mar. 22, 2000 (15966-737); U.S. Ser. No. 60/191,947, filed Mar. 24, 2000 (15966-738); U.S. Ser. No. 60/192,665, filed Mar. 28, 2000 (15966-739); U.S. Ser. No. 60/192,657, filed Mar. 28, 2000 (15966-740); U.S. Ser. No. 60/192,984, filed Mar. 28, 2000 (15966-741); U.S. Ser. No. 60/192,664), filed Mar. 28, 2000 (15966-742); U.S. Ser. No. 60/192,836, filed Mar. 29, 2000 (15966-743), and U.S. Ser. No. 60/193,843, filed Mar. 31, 2000 (15966-741A), which are incorporated herein by reference in their entirety.

### FIELD OF THE INVENTION

The invention generally relates to nucleic acids and polypeptides encoded therefrom.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The invention is based, in part, upon the discovery of novel polynucleotide sequences encoding novel polypeptides.

Accordingly, in one aspect, the invention provides an isolated nucleic acid molecule that includes the sequence of SEQ ID NO: 1, 3, 5, 7, 9, 11, 13, 15, 17, 19, 21 or 23 or a fragment, homolog, analog or derivative thereof. The nucleic acid can include, e.g., a nucleic acid sequence encoding a polypeptide at least 85% identical to a polypeptide that includes the amino acid sequences of SEQ ID NO: 2, 4, 6, 8, 10, 12, 14, 16, 18, 20, 22 or 24. The nucleic acid can be, e.g., a genomic DNA fragment, or a cDNA molecule.

Also included in the invention is a vector containing one or more of the nucleic acids described herein, and a cell containing the vectors or nucleic acids described herein.

The invention is also directed to host cells transformed with a vector comprising any of the nucleic acid molecules described above.

In another aspect, the invention includes a pharmaceutical composition that includes a NOVX nucleic acid and a pharmaceutically acceptable carrier or diluent.

In a further aspect, the invention includes a substantially purified NOVX polypeptide, e.g., any of the NOVX polypeptides encoded by a NOVX nucleic acid, and fragments, homologs, analogs, and derivatives thereof. The invention also includes a pharmaceutical composition that includes a NOVX polypeptide and a pharmaceutically acceptable carrier or diluent.

In still a further aspect, the invention provides an antibody that binds specifically to a NOVX polypeptide. The antibody can be, e.g., a monoclonal or polyclonal antibody, and fragments, homologs, analogs, and derivatives thereof. The invention also includes a pharmaceutical composition including NOVX antibody and a pharmaceutically acceptable carrier or diluent. The invention is also directed to isolated antibodies that bind to an epitope on a polypeptide encoded by any of the nucleic acid molecules described above.

The invention also includes kits comprising any of the pharmaceutical compositions described above.

The invention further provides a method for producing a NOVX polypeptide by providing a cell containing a NOVX nucleic acid, e.g., a vector that includes a NOVX nucleic acid, and culturing the cell under conditions sufficient to express the NOVX polypeptide encoded by the nucleic acid. The expressed NOVX polypeptide is then recovered from the cell. Preferably, the cell produces little or no endogenous NOVX polypeptide. The cell can be, e.g., a prokaryotic cell or eukaryotic cell.

The invention is also directed to methods of identifying a NOVX polypeptide or nucleic acid in a sample by contacting the sample with a compound that specifically binds to the polypeptide or nucleic acid, and detecting complex formation, if present.

The invention further provides methods of identifying a compound that modulates the activity of a NOVX polypeptide by contacting a NOVX polypeptide with a compound and determining whether the NOVX polypeptide activity is modified.

The invention is also directed to compounds that modulate NOVX polypeptide activity identified by contacting a NOVX polypeptide with the compound and determining whether the compound modifies activity of the NOVX polypeptide, binds to the NOVX polypeptide, or binds to a nucleic acid molecule encoding a NOVX polypeptide.

In another aspect, the invention provides a method of determining the presence of or predisposition of a NOVX-associated disorder in a subject. The method includes providing a sample from the subject and measuring the amount of NOVX polypeptide in the subject sample. The amount of NOVX polypeptide in the subject sample is then compared to the amount of NOVX polypeptide in a control sample. An alteration in the amount of NOVX polypeptide in the subject protein sample relative to the amount of NOVX polypeptide in the control protein sample indicates the subject has a tissue proliferation-associated condition. A control sample is preferably taken from a matched individual, i.e., an individual of similar age, sex, or other general condition but who is not suspected of having a tissue proliferation-associated condition. Alternatively, the control sample may be taken from the subject at a time when the subject is not suspected of having a tissue proliferation-associated disorder. In some embodiments, the NOVX is detected using a NOVX antibody.

In a further aspect, the invention provides a method of determining the presence of or predisposition of a NOVX-associated disorder in a subject. The method includes providing a nucleic acid sample, e.g., RNA or DNA, or both, from the subject and measuring the amount of the NOVX nucleic acid in the subject nucleic acid sample. The amount of NOVX nucleic acid sample in the subject nucleic acid is then compared to the amount of a NOVX nucleic acid in a control sample. An alteration in the amount of NOVX nucleic acid in the sample relative to the amount of NOVX in the control sample indicates the subject has a NOVX-associated disorder.

In a still further aspect, the invention provides a method of treating or preventing or delaying a NOVX-associated disorder. The method includes administering to a subject in which such treatment or prevention or delay is desired a NOVX nucleic acid, a NOVX polypeptide, or a NOVX antibody in an amount sufficient to treat, prevent, or delay a NOVX-associated disorder in the subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their polypeptides. The sequences are collectively referred to as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table 1 provides a summary of the NOVX nucleic acids and their encoded polypeptides. Example 1 provides a description of how the novel nucleic acids were identified.

TABLE 1
Sequences and Corresponding SEQ ID Numbers

| | | SEQ ID | | |
|---|---|---|---|---|
| | | NO | | |
| NOVX | | (nucleic | SEQ ID NO | |
| Assignment | Internal Identification | acid) | (polypeptide) | Homology |
| 1 | 2396e7_clone | 1 | 2 | Chloride ion channel |
| 2 | Cit978skb_139p6_A | 3 | 4 | Fatty acid-binding protein |
| | | | | (FABP) |
| 3 | 94115520_EXT | 5 | 6 | Insulin-like growth factor |
| 4 | GB_ACC#360_L_9_A | 7 | 8 | Cytokeratin-18 |
| 5 | 21426654_EXT | 9 | 10 | Metallocarboxypeptidase |
| 6 | AL031704_A | 11 | 12 | Mast cell protease-6 |
| 7 | 71768093_A | 13 | 14 | Sulfate anion transporter |
| 8 | 416_d_14_A | 15 | 16 | Cytostatin |
| 9 | 416_d_14_B | 17 | 18 | Cytostatin |
| 10 | GM_38019075_A | 19 | 20 | Chemokine receptor |
| 11 | CG54656-05 | 21 | 22 | Chemokine receptor |
| 12 | 32338334_1 | 23 | 24 | Carboxypeptidase |

Where FABP indicates a Fatty Acid Binding Protein

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

For example, NOV1 is homologous to members of the chloride channel family of proteins that are important in maintaining physiological ion balance and neuronal signal transduction. Thus, the NOV1 nucleic acids, polypeptides, antibodies and related compounds according to the invention will be useful in therapeutic and diagnostic applications in disorders characterized by altered ion regulation and neural signaling, e.g. cystic fibrosis, arrythmia seen in long QT syndrome, Dent's disease, Bartter's syndrome, bronchitis and sinusitis.

Also, NOV2 is homologous to a family of fatty acid-binding proteins important in keratinocyte differentiation. Thus NOV2 nucleic acids, polypeptides, antibodies and related compounds according to the invention will be useful in therapeutic and diagnostic applications in disorders characterized by aberrant keratinocyte differentiation, e.g. squamous cell carcinoma and lesional psoriatic skin.

Further, NOV3 is homologous to a family of insulin-like growth factor-binding proteins important in cell proliferation and differentiation. Thus, the NOV3 nucleic acids and polypeptides, antibodies and related compounds according to the invention will be useful in therapeutic and diagnostic applications in proliferative and apoptotic disorders, e.g. cancer, Alzheimer's disease, and obesity.

Also, NOV4 is homologous to the cytokeratin-18 family of proteins important in cytoskeletal stability in keratinocytes and other cell types. Thus, NOV4 nucleic acids, polypeptides, antibodies and related compounds according to the invention will be useful in therapeutic and diagnostic applications in disorders of the liver, pancreas and intestine, e.g. chronic hepatitis and drug-induced hepatotoxicity.

Additionally, NOV5 and NOV12 are homologous to the carboxypeptidase family of proteins important in peptide processing. Thus NOV5 and NOV12 nucleic acids, polypeptides, antibodies and related compounds according to the invention will be useful in therapeutic and diagnostic applications in metabolic disorders of the pancreas, e.g. acute pancreatitis.

Also, NOV6 is homologous to the mast cell protease-6 family of proteins important in mast cell activation and migration. Thus NOV6 nucleic acids, polypeptides, antibodies and related compounds according to the invention will be useful in therapeutic and diagnostic applications in disorders of the immune system, e.g. infectious inflammatory peritonitis.

Further, NOV7 is homologous to members of the sulfate anion channel family of proteins that are important in maintaining physiological ion balance and neuronal signal transduction. Thus, the NOV7 nucleic acids, polypeptides, antibodies and related compounds according to the invention will be useful in therapeutic and diagnostic applications in disorders characterized by altered sulfate anion regulation and neural signaling, e.g. Pendred syndrome, diastrophic dysplasia and other skeletal dysplasias.

Still further, NOV8-9 are homologous to a family of cytostatin-like proteins that are important in modulation of cell shape and motility by controlling cell interactions with the extracellular matrix. Thus, NOV8-9 nucleic acids and polypeptides, antibodies and related compounds according to the invention will be useful in therapeutic and diagnostic applications in disorders characterized by altered cell shape, motility, and apoptosis, e.g. cancer and ischemic injury.

Finally, NOV10-11 are homologous to the chemokine receptor family of proteins that are important in neuronal signal transduction and lymphocyte chemoattraction. Thus, NOV10-11 nucleic acids and polypeptides, antibodies and related compounds according to the invention will be useful in therapeutic and diagnostic applications in disorders characterized by altered immune response to injury and infection, e.g. AIDS, acute lung injury, adult respiratory distress syndrome, and multiple sclerosis.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell motility, cell proliferation, hematopoiesis, wound healing and angiogenesis.

Additional utilities for the NOVX nucleic acids and polypeptides according to the invention are disclosed herein.

NOV1

A NOV1 sequence according to the invention includes a nucleic acid sequence encoding a polypeptide related to the chloride channel family of proteins. A NOV1 nucleic acid is found on human chromosome 19. A NOV1 nucleic acid and its encoded polypeptide includes the sequences shown in Table 2. The disclosed nucleic acid (SEQ ID NO:1) is 739 nucleotides in length and contains an open reading frame (ORF) that begins with an ATG initiation codon at nucleotides 1-3 and ends with a TAA stop codon at nucleotides 737-739. The representative ORF encodes a 246 amino acid polypeptide (SEQ ID NO:2) with a predicted molecular weight of 28,017.3 daltons (Da). PSORT analysis of a NOV1 polypeptide predicts a plasma membrane protein with a certainty of 0.7900. SIGNALP analysis suggests the presence of a signal peptide with the most likely cleavage site occuring between positions 53 and 54 in SEQ ID NO.: 2.

TABLE 2

| | |
|---|---|
| ATGGCATTGTCGATGCCACTGAACAAGTTGAAGGAG | (SEQ ID NO.:1) |
| GAAGACAAAGAGCCCCTCCTTGAGCTCTGGGTCAAG | |
| GCTGTCAGTGATGGTGAAAGCACAGGAATCTGCCTT | |
| TTTTCCCAGAGATTCCTCATGATTCTTTGGCTCAAA | |
| GGAGTTGTCTTCAGTGTCACAACTGTTGATCTGAAA | |
| AGGAAACCTGCAGATCTGCAAAACAAGGCTCCTGGG | |
| AACCACCCACCACTTATAACTTCAACAGTGAAGTCA | |
| AATAAGATTGAGGAAGCTCCTGAAGAAGTCTTATGT | |
| CCTCCCAAGTACTTAAAGCTTTCACCAAAACACCCA | |
| GAATCAAATACTGCTGGAATGGACATCTTTGCCAAA | |
| TTCTCTGCATACATCAAGAATTCAAGGCCAGAGGTT | |
| AATGAAGCATTAGTGAAGCATCTCTTAAAAACCCTG | |
| CAGAAAATGGAATATCTGAATTCTCCTCTCCCTGAT | |
| GAAATTGATGAAAATAGCATGCAGGACACTAAGTTT | |
| TCTACACATAAATTTCTGAATGGCAATAAAATGGCA | |
| TTAGCTGATTGCCATCTGCTGCCCAAACTGCATATT | |
| GTCAAAAAAAAAGAAAAATATAGAAAATATAAAAAT | |
| ATAGAAAAAAAAGGAATGACTGGCATCTGGAGATAC | |
| CTAACGAATACAAGTAGTAGGGATATGTTCAACAAT | |
| ACCTGTCCCAATGATAAAGAGATTGAAATAGCAGCA | |
| GAAACAGTTAATGTAGTAA | |
| MALSMPLNKLKEEDKEPLLELWVKAVSDGESTGICL | (SEQ ID NO.:2) |
| FSQRFLMILWLKGVVFSVTTVDLKRKPADLQNKAPG | |
| NHPPLITSTVKSNKIEEAPEEVLCPPKYLKLSPKHP | |
| ESNTAGMDIFAKFSAYIKNSRPEVNEALVKHLLKTL | |
| QKMEYLNSPLPDEIDENSMQDTKFSTHKFLNGNKMA | |
| LADCHLLPKLHIVKKKEKYRKYKNIEKKGMTGIWRY | |
| LTNTSSRDMFNNTCPNDKEIEIAAETVNVV | |

A NOV1 nucleic acid has a high degree of homology (92% identity) with a human chloride channel protein P64-like mRNA (CC64; GenBank Accession No.: AK001624), as is shown in Table 3. A NOV1 polypeptide also has homology (78% identity, 85% similarity) with an intracellular human chloride channel polypeptide (ICCP; EMBL Accession No.: AAF19055), as is shown in Table 4.

TABLE 3

| | | |
|---|---|---|
| NOV1: 252 | aaataagattgaggaagctcctgaagaagtcttatgtcctcccaagtacttaaagctttc 311 | (SEQ ID NO.: 25) |
| | \|\|\|\|\|\|\|\|\|\|\|\|\|\|\|\| \|\| \|\|\|\|\|\|\|\|\|\|\|\|\|\|\| \|\|\|\|\|\|\|\|\|\|\|\|\|\|\|\|\|\|\|\|\|\|\| | |
| CC64: 436 | aaataagattgaggaatttcttgaagaagtcttatgccctcccaagtacttaaagctttc 495 | (SEQ ID NO.: 26) |
| NOV1: 312 | accaaaacacccagaatcaaatactgctggaatggacatctttgccaaattctctgcata 371 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| CC64: 496 | accaaaacacccagaatcaaatactgctggaatggacatctttgccaaattctctgcata 555 | |
| NOV1: 372 | catcaagaattcaaggccagaggttaatgaagcattagtgaagcatctcttaaaaaccct 431 | |
| | \|\|\|\|\|\|\|\|\|\|\|\|\|\|\|\|\|\|\|\|\|\| \|\|\|\|\|\|\|\|\|\| \| \| \|\| \| \|\|\|\| \| \|\|\|\|\|\|\|\| | |
| CC64: 556 | tatcaagaattcaaggccagaggctaatgaagcactggagaggggtctcctgaaaaccct 615 | |
| NOV1: 432 | gcagaaaatg---gaatatctgaattctcctctccctgatgaaattgatgaaaatagcat 488 | |
| | \|\|\|\|\|\|\| \|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\| | |
| CC64: 616 | gcagaaactggatgaatatctgaattctcctctccctgatgaaattgatgaaaatagtat 675 | |
| NOV1: 489 | gcaggacactaagttttctacacataaatttctgaatggcaataaaatggcattagctga 548 | |
| | \| \|\|\|\|\|\| \|\|\|\|\|\|\|\|\|\|\|\|\| \|\|\|\|\|\|\|\|\|\| \|\|\|\|\|\|\|\| \|\|\|\|\| \|\|\|\|\|\|\|\|\|\| | |
| CC64: 676 | ggaggacataaagttttctacacgtaaatttctggatggcaatgaaatgacattagctga 735 | |
| NOV1: 549 | ttgccatctgctgcccaaactgcatattgtc 579 | |
| | \|\|\|\| \| \|\|\|\|\|\|\|\|\|\|\|\|\|\|\|\|\|\|\|\|\|\|\|\| | |
| CC64: 736 | ttgcaacctgctgcccaaactgcatattgtc 766 | |

TABLE 4

| | | |
|---|---|---|
| NOV1: | 1 MALSMPLNKLKEEDKEPLLELWVKAVSDGESTGICLFSQRFLMILWLKGVVFSVTTVDLK 60 | (SEQ ID NO.: 2) |
| | \|\|\|\|\|\|\|\| \|\|\|\|\|\|\|\|\|+\|\|+\|\|\| \|\|\|\|\| \| \| \|\|\|\| \|\|\|\|\|\|\|\|\|\|\|\|\|\|\|\|\|\| | |
| ICCP: | 1 MALSMPLNGLKEEDKEPLIELFVKAGSDGESIGNCPFSQRLFMILWLKGVVFSVTTVDLK 60 | (SEQ ID NO.: 27) |
| NOV1: | 61 RKPADLQNKAPGNHPPLIT--STVKS--NKTEEAPEEVLCPPKYLKLSPKHPESNTAGMD 116 | |
| | \|\|\|\|\|\|\|\| \|\|\| \|\|\| \|\| \| \|\|+ \|\|\|\|\| \|\|\|\|\|\|\|\|\|\|\|\|\|\|\|\|\|\|\|\|\|\|\|\|\| | |
| ICCP: | 61 RKPADLQNLAPGTHPPFITFNSEVKTDVNKIEEFLEEVLCPPKYLKLSPKHPESNTAGMD 120 | |
| NOV1: | 117 IFAKFSAYIKNSRPEVNEALVKHLLKTLQKM-EYLNSPLPDEIDENSMQDTKFSTHKFLN 175 | |
| | \|\|\|\|\|\|\|\|\|\|\|\|\|\|\| \|\|\|\| + \|\|\|\|\|\|\|+ \|\|\|\|\|\|\|\|\|\|\|\|\|\|\|\|+\| \|\|\|\| +\|\| | |
| ICCP: | 121 IFAKFSAYIKNSRPEANEALERGLLKTLQKLDEYLNSPLPDEIDENSMEDIKFSTRRFLD 180 | |
| NOV1: | 176 GNKMALADCHLLPKLHIVKKKEKYRKYKNIE-KKGMTGIWRYLTNTSSRDMFNNTCPNDK 234 | |
| | \|++\| \|\|\|\|+\|\|\|\|\|\|\|\|\| \| \|\|+\| + \|\|\|\|\|\|\|\|\|\|\|\| \|\|\| \| \|\|\|\|+\|\| | |
| ICCP: | 181 GDEMTLADCNLLPKLHIVKVVAK--KYRNFDIPKGMTGIWRYLTNAYSRDEFTNTCPSDK 238 | |
| NOV1: | 235 EIEIAAETVNVV 246 | |
| | \|+\|\|\| \| | |
| ICCP: | 239 EVEIAYSDV 247 | |

Where | indicates identity and + indicates similarity.

Transporters, channels, and pumps that reside in cell membranes are key to maintaining the right balance of ions in cells, and are vital for transmitting signals from nerves to tissues. The consequences of defects in ion channels and transporters are diverse, depending on where they are located and what their cargo is. In the heart, defects in potassium channels do not allow proper transmission of electrical impulses, resulting in the arrythmia seen in long QT syndrome. In the lungs, failure of a sodium and chloride transporter found in epithelial cells leads to the congestion of cystic fibrosis, while one of the most common inherited forms of deafness, Pendred syndrome, looks to be associated with a defect in a sulphate transporter.

Chloride channels (CLC) perform important roles in the regulation of cellular excitability, in transepithelial transport, cell volume regulation, and acidification of intracellular organelles. This variety of functions requires a large number of different chloride channels that are encoded by genes belonging to several unrelated gene families. The CLC family of chloride channels has nine known members in mammals that show a differential tissue distribution and function both in plasma membranes and in intracellular organelles. CLC proteins have about 10-12 transmembrane domains. They probably function as dimers and may have two pores. The functional expression of channels altered by site-directed mutagenesis has led to important insights into their structure-function relationship. Their physiological relevance is obvious from three human inherited diseases (myotonia congenita, Dent's disease and Bartter's syndrome) that result from mutations in some of their members and from a knock-out mouse model (See Jentsch et al.,1999, Pflugers Arch. 437:783).

Recent studies of hereditary renal tubular disorders have facilitated the identification and roles of chloride channels and cotransporters in the regulation of the most abundant anion, Cl—, in the ECF. Thus, mutations that result in a loss of function of the voltage-gated chloride channel, CLC-5, are associated with Dent's disease, which is characterized by low-molecular weight proteinuria, hypercalciuria, nephrolithiasis, and renal failure. Mutations of another voltage-gated chloride channel, CLC-Kb, are associated with a form of Bartter's syndrome, whereas other forms of Bartter's syndrome are caused by mutations in the bumetanide-sensitive sodium-potassium-chloride cotransporter (NKCC2) and the potassium channel, ROMK. Finally, mutations of the thiazide-sensitive sodium-chloride cotransporter (NCCT) are associated with Gitelman's syndrome (See Thakker, 1999, Adv Nephrol. Necker Hosp. 29:289). These studies have helped to elucidate some of the renal tubular mechanisms regulating mineral homeostasis and the role of chloride channels.

A more prominent case of chloride channel dysfunction is cystic fibrosis. Cystic fibrosis (CF) is a genetic disease with multisystem involvement in which defective chloride transport across membranes causes dehydrated secretions. Cystic fibrosis (CF) affects approximately 1 in 2000 people making it one of the commonest fatal, inherited diseases in the Caucasian population. Dysfunction of the cystic fibrosis transmembrane conductance regulator (CFTR) Cl— channel is also associated with a wide spectrum of diseases (See Hwang & Sheppard, 1999, Trends Pharmacol. Sci. 20:448). The protein encoded by the CF gene, the cystic fibrosis transmembrane conductance regulator (CFTR), functions as a cyclic adenosine monophosphate-regulated chloride channel. The ability to detect CFTR mutations has led to the recognition of its association with a variety of conditions, including chronic bronchitis, sinusitis with nasal polyps, pancreatitis, and, in men, infertility (Choudari et al, 1999, Gastroenterol. Clin. North Am. 28:543). In the search for modulators of CFTR, pharmacological agents that interact directly with the CFTR Cl— channel have been identified. Some agents stimulate CFTR by interacting with the nucleotide-binding domains that control channel gating, whereas others inhibit CFTR by binding within the channel pore and preventing Cl— permeation. Knowledge of the molecular pharmacology of CFTR might lead to new treatments for diseases caused by the dysfunction of CFTR.

NOV1 represents a new member of the chloride channel family. NOV1 can be used as a marker for human chromosome 19. NOV1 is useful in determining changes in expression of genes contained within the chloride channel protein family. NOV1 satisfies a need in the art by providing new diagnostic or therapeutic compositions useful in the treatment of disorders associated with alterations in the expression of members of chloride channel-associated proteins. NOV1 nucleic acids, polypeptides, antibodies, and other compositions of the present invention are useful in the treatment and/or diagnosis of a variety of diseases and pathologies, including by way of nonlimiting example, those involving cystic fibrosis, congenital myotonia, Dent disease, an X-linked renal tubular disorder, leukoencephalopathy, malignant hyperthermia, hypertension, arrythmia seen in long QT syndrome, Dent's disease, Bartter's syndrome, bronchitis, sinusitis and other pathologies and disorders.

NOV2

A NOV2 sequence according to the invention includes a nucleic acid sequence encoding a polypeptide related to the fatty acid-binding protein family of proteins. A NOV2 nucleic acid is found on human chromosome 5. A NOV2 nucleic acid and its encoded polypeptide includes the sequences shown in Table 5. The disclosed nucleic acid (SEQ ID NO:3) is 550 nucleotides in length and contains an open reading frame (ORF) that begins with an ATG initiation codon at nucleotides 27-29 and ends with a TAA stop codon at nucleotides 543-545. The representative ORF encodes a 172 amino acid polypeptide (SEQ ID NO:4) with a predicted molecular weight of 19,464.4 Da. PSORT analysis of a NOV2 polypeptide predicts a mitochondrial matrix protein with a certainty of 0.3600. SIGNALP analysis suggests the lack of a signal peptide.

TABLE 5

| | |
|---|---|
| TCTGAGGACACAGCCACACTCTTGTCATGCCATTGCCCTTCTATTCTTTCCTTATAAC | (SEQ ID NO.: 3) |
| ATCATGTAAGAGGGCACAGCATGTTTCCCATGCTGGACCCTGCTCTGCTCACTCCAC | |
| ACACCTTCTGACACCCACCATGGACACTGTTCAGCAACTGGAAGAAAGAGGGCACC | |
| TGATGGACAGCAAAGGCTTTGATGAAAATAAATACATGAAGGAACTAGGAGTGGGA | |
| CTAGCCCTCTGCGAAAAAAAGGGTGCTATGGCCAAAAAAGATTGTATTAGCTTTTTT | |
| GATGGCAAAAACCTCACCATAAAAATGGAGAGTACTTTAAAATCATACAGTTTTCTC | |
| ACACTCAGGGGAGGGAAATTCAAAGAAACTACAGGTGACGGCAGAAAAACTCAGA | |
| CTTGCACCTTTACATATGGCACATTGGTTCGACATCAGAAGTGGAATGGAAAGGAAG | |
| GCAAAATAAGAAAATTGAAAGACAGGAAATTAGTGGTGGACTGCATCATAAACAAT | |
| GTCACCTGTACTCAGATCTATGAAAAAGTAGAATAAAAACT | |
| MPLPFYSFLITSCKRAQHVSHAGPCSAHSTHLLTPTMDTVQQLEERGHLMDSKGFDENK | (SEQ ID NO.: 4) |
| YMKELGVGLALCEKKGAMAKKDCISFFDGKNLTIKMESTLKSYSFLTLRGGKFKETTGD | |
| GRKTQTCTFTYGTLVRHQKWNGKEGKIRKLKDRKLVVDCIINNVTCTQIYEKVE | |

A NOV2 nucleic acid has a high degree of homology (99% identity) with an uncharacterized region of human chromosome 5, including the clone CTB-139P6 (CHR5; GenBank Accession No.: AC010293), as is shown in Table 6. A NOV2 polypeptide has homology (71% identity, 79% similarity) with a human epidermal fatty acid-binding protein polypeptide (FABP; EMBL Accession No.: Q01469), as is shown in Table 7. A NOV2 polypeptide also has homology (71% identity, 79% similarity) with a human melanogenic inhibitor polypeptide (hMI; PatP Accession No.: R55866) as is shown in Table 8.

TABLE 6

| | | |
|---|---|---|
| NOV2: | 1 tctgaggacacagccacactcttgtcatgccattgcccttctattctttccttataacat 60 | (SEQ ID NO.: 3) |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Chr5: | 19410 tctgaggacacagccacactcttgtcatgccattgcccttctattctttccttataacat 19469 | (SEQ ID NO.: 28) |
| NOV2: | 61 catgtaagagggcacagcatgtttcccatgctggaccctgctctgctcactccacacacc 120 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Chr5: | 19470 catgtaagagggcacagcatgtttcccatgctggaccctgctctgctcactccacacacc 19529 | |
| N0V2: | 121 ttctgacacccaccatggacactgttcagcaactggaagaaagagggcacctgatggaca 180 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Chr5: | 19530 ttctgacacccaccatggacactgttcagcaactggaagaaagagggcacctgatggaca 19589 | |
| NOV2: | 181 gcaaaggctttgatgaa-aataaatacatgaaggaactaggagtgggactagccctctgc 239 | |
| | \|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\| | |
| Chr5: | 19590 gcaaaggctttgatgaataataaatacatgaaggaactaggagtgggactagccctctgc 19649 | |
| NOV2: | 240 gaaaaaaagggtgctatggccaaaaaagattgtattagcttttttgatggcaaaaacctc 299 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Chr5: | 19650 gaaaaaaagggtgctatggccaaaaaagattgtattagcttttttgatggcaaaaacctc 19709 | |
| N0V2: | 300 accataaaaatggagagtactttaaaatcatacagttttctcacactcaggggagggaaa 359 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Chr5: | 19710 accataaaaatggagagtactttaaaatcatacagttttctcacactcaggggagggaaa 19769 | |
| NOV2: | 360 ttcaaagaaactacaggtgacggcagaaaaactcagacttgcacctttacatatggcaca 419 | |
| | \|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\| | |
| Chr5: | 19770 ttcaaagaaactacaggtgacggcagaaaaactcagac-tgcacctttacatatggcaca 19828 | |
| NOV2: | 420 ttggttcgacatcagaagtggaatggaaaggaaggcaaaataagaaaattgaaagacagg 479 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Chr5: | 19829 ttggttcgacatcagaagtggaatggaaaggaaggcaaaataagaaaattgaaagacagg 19888 | |
| NOV2: | 480 aaattagtggtggactgcatcataaacaatgtcacctgtactcagatctatgaaaaagta 539 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Chr5: | 19889 aaattagtggtggactgcatcataaacaatgtcacctgtactcagatctatgaaaaagta 19948 | |
| NOV2: | 540 gaataaaaact 550 | |
| | \|\|\|\|\|\|\|\|\|\|\| | |
| Chr5: | 19949 gaataaaaact 19959 | |

TABLE 7

| | | |
|---|---|---|
| NOV2: | 1 MDTVQQLEERGHLMDSKGFDENKYMKELGVGLALCEKKGAMAKKDCISFFDGKNLTIKME 60 | (SEQ ID NO.: 29) |
| | \| \|\|\|\|\|\| \| \|+\|\|\|\|\|\|\| \|\|\|\|\|\|\|\|+\|\| \| \|\|\|\|\| \|\|\| \|\|\|\|\|\|\|\| \| | |
| FABP: | 1 MATVQQLEGRNRLVDSKGFDE--YMKELGVGIAL-RKMGAMAKPDCIITCDGKNLTIKTE 57 | (SEQ ID NO.: 30) |
| NOV2: | 61 STLKSYSFLTLRGGKFKETTGDGRKTQT-CTFTYGTLVRHQKWNGKEGKI-RKLKDRKLV 118 | |
| | \|\|\|\|+ \| \| \|\|+\|\|\| \|\|\|\|\|\|\| \| \|\| \| \|\|+\|\|+\|+\|\|\| \| \|\|\|\|\| \|\|\| | |
| FABP: | 58 STLKTTQFSCTLGEKFEETTADGRKTQTVCNFTDGALVQHQEWDGKESTITRKLKDGKLV 117 | |
| NOV2: | 119 VDCIINNVTCTQIYEKVE 136 | |
| | \|+\|++\|\|\|\|\|\|+\|\|\|\|\|\| | |
| FABP: | 118 VECVMNNVTCTRIYEKVE 135 | |

Where | indicates identity and + indicates similarity.

TABLE 8

| | | |
|---|---|---|
| NOV2: | 1 MDTVQQLEERGHLMDSKGFDENKYMKELGVGLALCEKKGAMAKKDCISFFDGKNLTIKME 60 | (SEQ ID NO.: 29) |
| | \| \|\|\|\|\|\| \| \|+\|\|\|\|\|\|\| \|\|\|\|\|\|\|\|+\|\| \| \|\|\|\|\| \|\|\| \|\|\|\|\|\|\|\| \| | |
| HMI: | 1 MATVQQLEGRWRLVDSKGFDE--YMKELGVGIAL-RKMGAMAKPDCIITCDGKNLTIKTE 57 | (SEQ ID NO.: 31) |
| NOV2: | 61 STLKSYSFLTLRGGKFKETTGDGRKTQT-CTFTYGTLVRHQKWNGKEGKI-RKLKDRKLV 118 | |
| | \|\|\|\|+ \| \| \|\|+\|\|\| \|\|\|\|\|\|\| \| \|\| \| \|\|+\|\|+\|+\|\|\| \| \|\|\|\|\| \|\|\| | |
| HMI: | 58 STLKTTQFSCTLGEKFEETTADGRKTQTVCNFTDGALVQHQEWDGKESTITRKLKDGKLV 117 | |
| NOV2: | 119 VDCIINNVTCTQIYEKVE 136 | |
| | \|+\|++\|\|\|\|\|\|+\|\|\|\|\|\| | |
| HMI: | 118 VECVMNNVTCTRIYEKVE 135 | |

Where | indicates identity and + indicates similarity.

Fatty acid metabolism in mammalian cells depends on a flux of fatty acids, between the plasma membrane and mitochondria or peroxisomes for beta-oxidation, and between other cellular organelles for lipid synthesis. The fatty acid-binding protein (FABP) family consists of small, cytosolic proteins believed to be involved in the uptake, transport, and solubilization of their hydrophobic ligands. Members of this family have highly conserved sequences and tertiary structures. Fatty acid-binding proteins were first isolated in the intestine (FABP2; OMIM-134640) and later found in liver (FABP1; OMIM-134650), striated muscle (FABP3; OMIM-134651), adipocytes (FABP4; OMIM-600434) and epidermal tissues (E-FABP; GDB ID:136450).

Epidermal fatty acid binding protein (E-FABP) was cloned as a novel keratinocyte protein by Madsen and co-workers from the skin of psoriasis patients (See Madsen et al.,1992, J. Invest. Dermatol. 99:299). Later using quantitative Western blot analysis, Kingma and colleagues have shown that in addition to the skin, bovine E-FABP is expressed in retina, testis, and lens (See Kingma et al., 1998, Biochemistry 37:3250). Since E-FABP was originally identified from the skin of psoriasis patients, it is also known as psoriasis-associated fatty acid-binding protein (PA-FABP). PA-FABP is a cytoplasmic protein, and is expressed in keratinocytes. It is highly up-regulated in psoriatic skin. It shares similarity to other members of the fatty acid-binding proteins and belongs to the fabp/p2/crbp/crabp family of transporter. PA-FABP is believed to have a high specificity for fatty acids, with highest affinity for c18 chain length. Decreasing the chain length or introducing double bonds reduces the affinity. PA-FABP may be involved in keratinocyte differentiation.

Immunohistochemical localization of the expression of E-FABP in psoriasis, basal and squamous cell carcinomas has been carried out in order to obtain indirect information, at the cellular level, on the transport of the fatty acids (See Masouye et al., 1996, Dermatology 192:208). E-FABP was localized in the upper stratum spinosum and stratum granulosum in normal and non-lesional psoriatic skin. In conrast, lesional psoriatic epidermis strongly expressed E-FABP in all suprabasal layers, like nonkeratinized oral mucosa. The basal layer did not express E-FABP reactivity in any of these samples. Accordingly, basal cell carcinomas were E-FABP negative whereas only well-differentiated cells of squamous cell carcinomas expressed E-FABP. This suggests that E-FABP expression is related to the commitment of keratinocyte differentiation and that the putative role of E-FABP should not be restricted to the formation of the skin lipid barrier. Since the pattern of E-FABP expression mimics cellular FA transport, our results suggest that lesional psoriatic skin and oral mucosa have a higher metabolism/transport for fatty acids than normal and non-lesional psoriatic epidermis.

NOV2 represents a new member of the fatty acid-binding protein family. NOV2 can be used as a marker for human chromosome 5. NOV2 is useful in determining changes in expression of genes contained within the fatty acid-binding protein family. NOV2 satisfies a need in the art by providing new diagnostic or therapeutic compositions useful in the treatment of disorders associated with alterations in the expression of members of fatty acid-binding protein associated proteins. NOV2 nucleic acids, polypeptides, antibodies, and other compositions of the present invention are useful in the treatment and/or diagnosis of a variety of diseases and pathologies, including by way of nonlimiting example, those involving psoriatic skin and cancer, e.g. basal and squamous cell carcinomas.

NOV3

A NOV3 sequence according to the invention includes a nucleic acid sequence encoding a polypeptide related to the insulin-like growth factor family of proteins. A NOV3 nucleic acid is found on human chromosome 10. A NOV3 nucleic acid and its encoded polypeptide includes the sequences shown in Table 9. The disclosed nucleic acid (SEQ ID NO:5) is 915 nucleotides in length and contains an open reading frame (ORF) that begins with an ATG initiation codon at nucleotides 1-3 and ends with a TGA stop codon at nucleotides 913-915. The representative ORF encodes a 304 amino acid polypeptide (SEQ ID NO:6) with a predicted molecular weight of 32,944.7 Da. A NOV3 polypeptide is likely to be detected in kidney, spleen, thyroid, brain and salivary gland. PSORT analysis of a NOV3 polypeptide predicts a secreted protein with a certainty of 0.8200. SIGNALP analysis suggests the presence of a signal peptide with the most likely cleavage site occuring between positions 30 and 31 in SEQ ID NO.: 6.

TABLE 9

| | |
|---|---|
| ATGCTGCCGCCGCCGCGGCCCGCAGCTGCCTTGGCGCTGCCTGTGCTCCTGCTACTG | (SEQ ID NO.: 5) |
| CTGGTGGTGCTGACGCCGCCCCCGACCGGCGCAAGGCCATCCCCAGGCCCAGATTA | |
| CCTGCGGCGCGGCTGGATGCGGCTGCTAGCGGAGGGCGAGGGCTGCGCTCCCTGCC | |
| GGCCAGAAGAGTGCGCCGCGCCGCGGGGCTGCCTGGCGGGCAGGGTGCGCGACGCG | |
| TGCGGCTGCTGCTGGGAATGCGCCAACCTCGAGGGCCAGCTCTGCGACCTGGACCCC | |
| AGTGCTCACTTCTACGGGCACTGCGGCGAGCAGCTTGAGTGCCGGCTGGACACAGG | |
| CGGCGACCTGAGCCGCGGAGAGGTGCCGGAACCTCTGTGTGCCTGTCGTTCGCAGA | |
| GTCCGCTCTGCGGGTCCGACGGTCACACCTACTCCCAGATCTGCCGCCTGCAGGAGG | |
| CGGCCCGCGCTCGGCCCGATGCCAACCTCACTGTGGCACACCCGGGGCCCTGCGAA | |
| TCGGGGCCCCAGATCGTGTCACATCCATATGACACTTGGAATGTGACAGGGCAGGA | |
| TGTGATCTTTGGCTGTGAAGTGTTTGCCTACCCCATGGCCTCCATCGAGTGGAGGAA | |
| GGATGGCTTGGACATCCAGCTGCCAGGGGATGACCCCCACATCTCTGTGCAGTTTAG | |
| GGGTGGACCCCAGAGGTTTGAGGTGACTGGCTGGCTGCAGATCCAGGCTGTGCGTC | |
| CCAGTGATGAGGGCACTTACCGCTGCCTTGGCCGCAATGCCCTGGGTCAAGTGGAG | |
| GCCCCTGCTAGCTTGACAGTGCTCACACCTGACCAGCTGAACTCTACAGGCATCCCC | |
| CAGCTGCGATCACTAAACCTGGTTCCTGAGGAGGAGGCTGAGAGTGAAGAGAATGA | |
| CGATTACTACTAG | |
| MLPPPRPAAALALPVLLLLLVVLTPPPTGARPSPGPDYLRRGWMRLLAEGEGCAPCRPEE | (SEQ ID NO.: 6) |
| CAAPRGCLAGRVRDACGCCWECANLEGQLCDLDPSAHFYGHCGEQLECRLDTGGDLSR | |
| GEVPEPLCACRSQSPLCGSDGHTYSQICRLQEAARARPDANLTVAHPGPCESGPQIVSHP | |
| YDTWNVTGQDVIFGCEVFAYPMASIEWRKDGLDIQLPGDDPHISVQFRGGPQRFEVTGW | |
| LQIQAVRPSDEGTYRCLGRNALGQVEAPASLTVLTPDQLNSTGIPQLRSLNLVPEEEAESE | |
| ENDDYY | |

A NOV3 nucleic acid has a high degree of homology (100% identity) with an uncharacterized region of human chromosome 10, including the clone RP11-108L7 (CHR10; GenBank Accession No.: AL133215), as is shown in Table 10. A NOV3 polypeptide has a high degree of homology (99% identity) with a human prostacyclin-stimulating factor-2 polypeptide (PSF2; PATP Accession No.: Y93650), as is shown in Table 11. The expression patterns of a NOV3 nucleic acid were analyzed by expression profiling, as is shown in Example 3.

TABLE 10

| | | |
|---|---|---|
| NOV3: | 1 atgctgccgccgccgcggcccgcagctgccttggcgctgcctgtgctcctgctactgctg 60 | (SEQ ID NO.: 32) |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| CHR10: | 35670 atgctgccgccgccgcggcccgcagctgccttggcgctgcctgtgctcctgctactgctg 35611 | (SEQ ID NO.: 33) |
| NOV3: | 61 gtggtgctgacgccgcccccgaccggcgcaaggccatccccaggcccagattacctgcgg 120 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| CHR10: | 35610 gtggtgctgacgccgcccccgaccggcgcaaggccatccccaggcccagattacctgcgg 35551 | |
| NOV3: | 121 cgcggctggatgcggctgctagcggagggcgagggctgcgctccctgccggccagaagag 180 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| CHR10: | 35550 cgcggctggatgcggctgctagcggagggcgagggctgcgctccctgccggccagaagag 35491 | |
| NOV3: | 181 tgcgccgcgccgcggggctgcctggcgggcagggtgcgcgacgcgtgcggctgctgctgg 240 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| CHR10: | 35490 tgcgccgcgccgcggggctgcctggcgggcagggtgcgcgacgcgtgcggctgctgctgg 35431 | |
| NOV3: | 241 gaatgcgccaacctcgagggccagctctgcgacctggaccccagtgctcacttctacggg 300 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| CHR10: | 35430 gaatgcgccaacctcgagggccagctctgcgacctggaccccagtgctcacttctacggg 35371 | |
| NOV3: | 301 cactgcggcgagcagcttgagtgccggctggacacaggcggcgacctgagccgcggagag 360 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| CHR10: | 35370 cactgcggcgagcagcttgagtgccggctggacacaggcggcgacctgagccgcggagag 35311 | |
| NOV3: | 361 gtgccggaacctctgtgtgcctgtcgttcgcagagtccgctctgcgggtccgacggtcac 420 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| CHR10: | 35310 gtgccggaacctctgtgtgcctgtcgttcgcagagtccgctctgcgggtccgacggtcac 35251 | |
| NOV3: | 421 acctactcccagatctgccgcctgcaggaggcggcccgcgctcggcccgatgccaacctc 480 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| CHR10: | 35250 acctactcccagatctgccgcctgcaggaggcggcccgcgctcggcccgatgccaacctc 35191 | |
| NOV3: | 481 actgtggcacacccggggccctgcgaatcggg 512 | |
| | \|\|\|\|\|\|\|\|\|\|\|\|\|\|\|\|\|\|\|\|\|\|\|\|\|\|\|\|\|\|\|\| | |
| CHR10: | 35190 actgtggcacacccggggccctgcgaatcggg 35159 | |

TABLE 11

| | | |
|---|---|---|
| NOV3: | 1 MLPPPRPAAALALPVLLLLLVVLTPPPTGARPSPGPDYLRRGWMRLLAEGEGCAPCRPEE 60 | (SEQ ID NO.: 6) |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| PSF2: | 1 MLPPPRPAAALALPVLLLLLVVLTPPPTGARPSPGPDYLRRGWMRLLAEGEGCAPCRPEE 60 | (SEQ ID NO.: 34) |
| NOV3: | 61 CAAPRGCLAGRVRDACGCCWECANLEGQLCDLDPSAHFYGHCGEQLECRLDTGGDLSRGE 120 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| PSF2: | 61 CAAPRGCLAGRVRDACGCCWECANLEGQLCDLDPSAHFYGHCGEQLECRLDTGGDLSRGE 120 | |
| NOV3: | 121 VPEPLCACRSQSPLCGSDGHTYSQICRLQEAARARPDANLTVAHPGPCESGPQIVSHPYD 180 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| PSF2: | 121 VPEPLCACRSQSPLCGSDGHTYSQICRLQEAARARPDANLTVAHPGPCESGPQIVSHPYD 180 | |
| NOV3: | 181 TWNVTGQDVIFGCEVFAYPMASIEWRKDGLDIQLPGDDPHISVQFRGGPQRFEVTGWLQI 240 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| PSF2: | 181 TWNVTGQDVIFGCEVFAYPMASIEWRKDGLDIQLPGDDPHISVQFRGGPQRFEVTGWLQI 240 | |
| NOV3: | 241 QAVRPSDEGTYRCLGRNALGQVEAPASLTVLTPDQLNSTGIPQLRSLNLVPEEEAESEEN 300 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| PSF2: | 241 QAVRPSDEGTYRCLARNALGQVEAPASLTVLTPDQLNSTGIPQLRSLNLVPEEEAESEEN 300 | |
| NOV3: | 301 DDYY 304 | |
| | \|\|\|\| | |
| PSF2: | 301 DDYY 304 | |

Where | indicates identity and + indicates similarity.

The insulin-like growth factor binding protein (IGFBP) family comprises six structurally distinct, but highly homologous proteins. They have been identified in serum and other biological fluids, tissue extracts, and cell culture media. cDNAs encoding human IGFBP-4, -5, and -6 have been cloned and expressed these BPs in yeast as ubiquitin (Ub)-IGFBP fusion proteins. Western ligand blotting with 1251-IGF II under non-reducing conditions of recombinant human (rh) IGFBP-containing yeast lysates reveals specific binding bands for IGFBP-4, -5, and -6 at apparent molecular masses of 24-26, 30-32, and 24-26 kDa, respectively, indicating processing of the fusion proteins. High-performance liquid chromatography-purified rhIGFBPs have virtually the same amino acid composition, amino acid number, and NH2-terminal sequences as the native BPs. Except for the affinity of rhIGFBP-6 for IGF I (Ka=8.5×10(8) M-1), the affinity constants of the three IGFBPs for IGF I and II lie between 1.7 and 3.3×10(10) M-1, i.e. 25-100 times higher than the IGF I and II affinities of the type I IGF receptor. When present in excess, rhIGFBP-4, -5, and -6 inhibit IGF I- and II-stimulated DNA and glycogen synthesis in human osteoblastic cells, but rhIGFBP-6 has only a weak inhibitory effect on IGF I in agreement with its relatively lower IGF I affinity constant. IGFBPs contribute to the control of IGF-mediated cell growth and metabolism. (See Kiefer et al., 1992, J. Biol. Chem. 267:12692.).

Insulin-like growth factor proteins are associated with cancer progression. The down-regulation of T1A12/mac25, a novel insulin-like growth factor binding-like protein related gene, is associated with disease progression in breast carcinomas. To define genes that are essential to the initiation and progression of breast cancer Burger and colleagues utilized subtractive hybridization and differential display cloning techniques and isolated over 950 cDNAs from breast cell-lines derived from matched normal and tumor tissue. Of these, 102 cDNAs were characterized by DNA sequencing and Northern blot analysis. GenBank searches showed that one of these genes, T1A12 is identical to mac25, an insulin-like growth factor-binding protein related gene. Antibodies generated against the C-terminal region of the T1A12/mac25 protein were used to investigate its expression in 60 primary breast tissues. Sections of 12 benign, 16 ductal carcinoma in situ and 32 infiltrating ductal carcinoma specimens were examined. Strong immunoperoxidase staining was observed in luminal epithelial cells of normal lobules and ducts, in apocrine cells of cysts and fibroadenomas. Moderate to weak protein expression was found in hyperplastic and DCIS cells, but no specific staining was detected in invasive carcinoma cells. FISH mapping using a PAC clone localized the T1A12/mac25 gene to 4q12-13. Microsatellite length polymorphism was studied using markers for 4q in paired normal and tumor breast tissues. Thirty-three per cent (10/30) of the samples were found to be polymorphic with D4S189 and D4S231 microsatellite markers and LOH was detected in 50% (5/10) of these informative samples. The data indicate that T1A12/mac25 expression is abrogated during breast cancer progression concomitant with loss of heterozygosity on chromosome 4q. T1A12/mac25 may therefore have a tumor suppressor-like function and its expression could indicate a disease with a more favorable status, having a better prognosis (See Burger et al., Oncogene 16:2459).

NOV3 represents a new member of the insulin-like growth factor family. NOV3 can be used as a marker for human chromosome 10. NOV3 is useful in determining changes in expression of genes contained within the insulin-like growth factor protein family. NOV3 satisfies a need in the art by providing new diagnostic or therapeutic compositions useful in the treatment of disorders associated with alterations in the expression of members of insulin-like growth factor-like protein associated proteins. NOV3 nucleic acids, polypeptides, antibodies, and other compositions of the present invention are useful in the treatment and/or diagnosis of a variety of diseases and pathologies, including by way of nonlimiting example, those involving cell proliferative disorders, e.g. cancer.

NOV4

A NOV4 sequence according to the invention includes a nucleic acid sequence encoding a polypeptide related to the cytokeratin-18 family of proteins. A NOV4 nucleic acid and its encoded polypeptide includes the sequences shown in Table 12. The disclosed nucleic acid (SEQ ID NO:7) is 1,299 nucleotides in length and contains an open reading frame (ORF) that begins with an ATG initiation codon at nucleotides 5-7 and ends with a TAA stop codon at nucleotides 1,286-1,288. The representative ORF encodes a 427 amino acid polypeptide (SEQ ID NO:8) with a predicted molecular weight of 48,096.8 Da. PSORT analysis of a NOV4 polypeptide predicts localization in the endoplasmic reticulum membrane with a certainty of 0.5500. SIGNALP analysis suggests the lack of a signal peptide. Putative untranslated regions upstream and downstream of the coding sequence are underlined in SEQ ID NO.: 7).

TABLE 12

| | |
|---|---|
| CAGCATGAGCTTCACCACTCCCTCCACCTTCTCCACCAACTACCAGTCCCTGGGCTC | (SEQ ID NO.: 7) |
| TGTCCAGCCGCCCAGCTATGGCACCTGGCCGGTCAGCAGCGCAGCCAGCATCTATGC | |
| AGGCACTGGGGGGCTTGGGTCCCAGATCTCCATGTCCTGTTCTACCAGTTCTGGGG | |
| CGGCTTGGGGTCTGGGGGCCTGGCCACAGAGATGGCTGGGGGTCTGGCAGAAATGG | |
| GGGGCATCCAGAATGAGAAGGAGACCATGCAAAGCCTGAACGACCACCTGGACTAC | |
| CTGGACAGAGTGAGGAACCTGGAGACCGAGAACTGGAGGCTGGAGAGCAAAATCC | |
| AGGAGTATCTGGAGAAGAGACCCCATGTCAGAGACTGGGGCCATTACTTCAAGACC | |
| ATCAAGGAACTGAGGGCTCAGATCTTCGCAAATACTGTGGACAATGTCCACATCATT | |
| CTGCAGATCGACAATGCCCGTCTTGCTGCTGATGACTTCAGAGTCAAGTATGAGACA | |
| GAGCTGGCCATGCGCCAGTCTGTGGAGAGCAACATCCATGGGCTCTGCAAGGTCATT | |
| GATGACACCAATGTCACTCTGCTGCAGCTGGAGACAGAGATGGGCGCTCTCAAGGA | |
| GGAGCTGCTCCTCATGAAGAAGAACCATGAAGAGGAAGTAAAAGGCTTGCAAGTCC | |
| AGATTGCCAACTCTGGGTTGGCCGTGGAGGTAGATGCCCCCAAATCTCAAGTCCTCG | |
| CCAAGGTCATGGCAGACATCAGGGCCCAATATGATGAGCTGTCTCAGAAGAACTCA | |
| GAGAAGCTAGGCAAGTACTGGTCTCAGCAGACTGAGGAGAGCACCACAGTGGTCAC | |
| CACACACTCTGCCAAGGTCAGAGCTGCTGAGATGACAACGGAGCTGAGACGTACAG | |
| TCCAGTGCTTGGAGATTGACCTGGACTCAATGAGAAATCGAAGACCAGCTTGGAG | |
| AACAGCCTGAGGGAGGTGGAGGCCCGCTACGCCCTGCAGATGGAGCAGCTCAACAG | |
| AATCCTGCTGTACTTGGAGTCAAAGCTGGCACAGAACTGGGCAGAGGGCCAGCGCA | |
| AGGTCCAGGAGTACAAGGACTTGCTGAACATCAGGGTCAAGCTGGAGGCTGAGATC | |
| GCCACCTACCGCCGCCTGCTGGAAGACAGCGAGGGCCTCAATCTTGGTGATGCCCTG | |
| GACAGCAGCAACTCCATGCAAACCATCCAAAAGACCACCACCCGCCAGATAGTGGA | |
| TAGCAAAGTGGTGTCTGAGATCAGTGACACCAAAGTTCTGAGACATTAAGCCAGCA | |
| GAAG | |
| MSFTTPSTFSTNYQSLGSVQPPSYGTWPVSSAASIYAGTGGLGSQISMSCSTSFWGGLGSG | (SEQ ID NO.: 8) |
| GLATEMAGGLAEMGGIQNEKETMQSLNDHLDYLDRVRINLETENWRLESKIQEYLEKRP | |
| HVRDWGHYFKTIKELRAQIFANTVDNVHIILQIDNARLAADDFRVKYETELAMRQSVES | |
| NIHGLCKVLDDTNVTLLQLETEMGALKEELLLMKKMIEEEVKGLQVQIANSGLAVEVDA | |
| PKSQVLAKVMADIRAQYDELSQKNSEKLGKYWSQQTEESTTVVTTHSAKVRAAEMTTE | |
| LRRTVQCLEIDLDSMRNLKTSLENSLREVEARYALQMEQLNRILLYLESKLAQNW | |
| AEGQRKVQEYKDLLNIRVKLEAEIATYRRLLEDSEGLNLGDALDSSNSMQTIQKTTTRQI | |
| VDSKVVSEISDTKVLRH | |

A NOV4 nucleic acid has a high degree of homology (90% identity) with a human keratin-18 mRNA (K-18; GenBank Accession No.: M26326), as is shown in Table 13. A NOV4 polypeptide has homology (82% identity, 89% similarity) with a human keratin 18 polypeptide (hK18; GenBank Accession No.: S05481), as is shown in Table 14.

TABLE 13

| | | | | |
|---|---|---|---|---|
| NOV4: | 1 | CAGCATGAGCTTCACCACTCCCTCCACCTTCTCCACCAACTACCAGTCCCTGGGCTCTGT | 60 | (SEQ ID NO.: 7) |
| | | \|\|\|\|\|\|\|\|\|\|\|\|\|\|\|\|\|\|\|\| \|\|\|\|\|\|\|\|\|\|\|\|\|\|\|\|\|\|\|\|\|\|\| \|\|\|\|\|\|\|\|\|\|\|\|\|\|\| | | |
| K-18: | 48 | CAGCATGAGCTTCACCACTCGCTCCACCTTCTCCACCAACTACCGGTCCCTGGGCTCTGT | 107 | (SEQ ID NO.: 35) |
| NOV4: | 61 | CCAGCCGCCCAGCTATGGCACCTGGCCGGTCAGCAGCGCAGCCAGCATCTATGCAGGCAC | 120 | |
| | | \|\|\|\| \|\|\|\|\|\|\|\|\|\| \|\|\| \|\| \|\|\|\|\|\|\|\|\|\|\| \|\|\|\|\|\| \|\|\|\|\|\|\|\|\|\|\|\|\|\|\|\| \| | | |
| K-18: | 108 | CCAGGCGCCCAGCTACGGCGCCCGGCCGGTCAGCAGCGCGGCCAGCGTCTATGCAGGCGC | 167 | |
| NOV4: | 121 | TGGGGGGCT-TGGGTCCCAGATCTCCATGTCCTGTTCTACCAGTTTCTGGGGCGGCTTGG | 179 | |
| | | \|\|\|\|\|\| \|\| \|\|\| \|\|\|\| \|\|\|\|\|\|\| \|\|\|\|\| \| \|\| \|\|\|\|\| \|\|\| \|\|\|\|\|\|\|\| \|\|\| | | |
| K-18: | 168 | TGGGGG-CTCTGGTTCCCGGATCTCCGTGTCCCGCTCCACCAGCTTCAGGGGCGGCATGG | 226 | |
| NOV4: | 180 | GGTCTGGGGGCCTGGCCACAGAGATGGCTGGGGGTCTGGCAGAAATGGGGGGCATCCAGA | 239 | |
| | | \|\|\|\| \|\|\|\|\|\|\|\|\|\|\|\|\|\| \| \|\|\| \|\| \|\|\|\|\|\|\|\|\|\|\|\|\| \|\|\|\|\|\| \|\|\|\|\|\|\|\|\|\| | | |
| K-18: | 227 | GGTCCGGGGGCCTGGCCACCGGGATAGCCGGGGGTCTGGCAGGAATGGGAGGCATCCAGA | 286 | |
| NOV4: | 240 | ATGAGAAGGAGACCATGCAAAGCCTGAACGACCACCTGGACT---ACCTGGACAGAGTGA | 296 | |
| | | \| \|\|\|\|\|\|\|\|\|\|\|\|\|\|\|\|\|\|\|\|\|\|\|\|\|\|\|\|\|\|\| \|\|\|\|\| \|\| \|\|\|\|\|\|\|\|\|\|\|\|\|\|\| | | |
| K-18: | 287 | ACGAGAAGGAGACCATGCAAAGCCTGAACGACCGCCTGGCCTCTTACCTGGACAGAGTGA | 346 | |
| NOV4: | 297 | GGAACCTGGAGACCGAGAACTGGAGGCTGGAGAGCAAAATCCAGGAGTATCTGGAGAAGA | 356 | |
| | | \|\|\| \|\|\|\|\|\|\|\|\|\|\|\|\|\|\|\| \|\|\|\|\|\|\|\|\|\|\|\|\|\|\|\|\|\|\|\|\| \|\|\|\| \| \|\|\|\|\|\|\|\|\| | | |
| K-18: | 347 | GGAGCCTGGAGACCGAGAACCGGAGGCTGGAGAGCAAAATCCGGGAGCACTTGGAGAAGA | 406 | |
| NOV4: | 357 | -G--ACCCCATGTCAGAGACTGGGGCCATTACTTCAAGACCATCAAGGAACTGAGGGCTC | 413 | |
| | | \| \|\|\|\|\|\| \|\|\|\|\|\|\|\|\|\|\|\| \|\|\|\|\|\|\|\|\|\|\|\|\|\|\| \|\|\|\| \|\|\|\| \|\|\|\|\|\|\|\|\|\| | | |
| K-18: | 407 | AGGGACCCCAGGTCAGAGACTGGAGCCATTACTTCAAGATCATCGAGGACCTGAGGGCTC | 466 | |
| NOV4: | 414 | AGATCTTCGCAAATACTGTGGACAATGTCCACATCATTCTGCAGATCGACAATGCCCGTC | 473 | |
| | | \|\|\|\|\|\|\|\|\|\|\|\|\|\|\|\|\|\|\|\|\|\|\|\|\|\|\| \|\| \|\|\|\| \|\|\|\|\|\|\|\|\|\| \|\|\|\|\|\|\|\|\|\|\|\|\| | | |
| K-18: | 467 | AGATCTTCGCAAATACTGTGGACAATGCCCGCATCGTTCTGCAGATTGACAATGCCCGTC | 526 | |
| NOV4: | 474 | TTGCTGCTGATGACTTCAGAGTCAAGTATGAGACAGAGCTGGCCATGCGCCAGTCTGTGG | 533 | |
| | | \|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| K-18: | 527 | TTGCTGCTGATGACTTTAGAGTCAAGTATGAGACAGAGCTGGCCATGCGCCAGTCTGTGG | 586 | |
| NOV4: | 534 | AGAGCAACATCCATGGGCTCTGCAAGGTCATTGATGACACCAATGTCACTCTGCTGCAGC | 593 | |
| | | \|\|\| \| \|\|\|\|\|\|\|\|\|\|\|\|\|\| \|\|\|\|\|\|\|\|\|\|\|\|\|\|\|\|\|\|\|\|\|\|\| \|\|\|\| \| \|\|\|\|\|\|\| | | |
| K-18: | 587 | AGAACGACATCCATGGGCTCCGCAAGGTCATTGATGACACCAATATCACACGACTGCAGC | 646 | |
| NOV4: | 594 | TGGAGACAGAGATGGGCGCTCTCAAGGAGGAGCTGCTCCTCATGAAGAAGAACCATGAAG | 653 | |
| | | \|\|\|\|\|\|\|\|\|\|\|\|\| \| \|\|\|\|\|\|\|\|\|\|\|\|\|\|\|\|\|\|\|\|\| \|\|\|\|\|\|\|\|\|\|\|\|\|\|\|\| \|\|\|\| | | |
| K-18: | 647 | TGGAGACAGAGATCGAGGCTCTCAAGGAGGAGCTGCTCTTCATGAAGAAGAACCACGAAG | 706 | |
| NOV4: | 654 | AGGAAGTAAAAGGCTTGCAAGTCCAGATTGCCAACTCTGGGTTGGCCGTGGAGGTAGATG | 713 | |
| | | \|\|\|\|\|\|\|\|\|\|\|\|\|\| \| \|\|\|\| \|\|\|\|\|\|\|\|\|\|\| \|\|\|\|\|\|\|\|\|\| \|\|\|\|\|\|\|\|\|\|\|\|\|\|\| | | |
| K-18: | 707 | AGGAAGTAAAAGGCCTACAAGCCCAGATTGCCAGCTCTGGGTTGACCGTGGAGGTAGATG | 766 | |
| NOV4: | 714 | CCCCCAAATCTCAAGTCCTCGCCAAGGTCATGGCAGACATCAGGGCCCAATATGATGAGC | 773 | |
| | | \|\|\|\|\|\|\|\|\|\|\|\|\| \| \|\|\|\|\|\|\|\|\|\| \|\|\|\|\|\|\|\|\|\|\|\|\|\| \|\|\|\|\|\|\|\|\|\|\|\|\| \|\|\|\| | | |
| K-18: | 767 | CCCCCAAATCTCAGGACCTCGCCAAGATCATGGCAGACATCCGGGCCCAATATGACGAGC | 826 | |
| NOV4: | 774 | TGTCTCAGAAGAACTCAGAGAAGCTAGGCAAGTACTGGTCTCAGCAGACTGAGGAGAGCA | 833 | |
| | | \|\| \|\|\| \|\|\|\|\|\|\| \|\|\|\| \|\|\|\|\|\| \|\|\|\|\|\|\|\|\|\|\|\|\|\|\|\|\|\|\|\| \|\|\|\|\|\|\|\|\|\|\| | | |
| K-18: | 827 | TGGCTCGGAAGAACCGAGAGGAGCTAGACAAGTACTGGTCTCAGCAGATTGAGGAGAGCA | 886 | |
| NOV4: | 834 | CCACAGTGGTCACCACACACTCTGCCAAGGTCAGAGCTGCTGAGATGACA---ACGGAGC | 890 | |
| | | \|\|\|\|\|\|\|\|\|\|\|\|\|\|\|\|\|\|\| \|\|\|\|\| \|\|\|\| \|\|\|\|\|\|\|\|\|\|\|\| \|\|\|\|\|\|\|\|\|\| \|\|\| | | |
| K-18: | 887 | CCACAGTGGTCACCACACAGTCTGCTGAGGTTGGAGCTGCTGAGACGACGCTCACAGAGC | 946 | |
| NOV4: | 891 | TGAGACGTACAGTCCAGTGCTTGGAGATTGACCTGGACTCAATGAGAAATCTGAAGACCA | 950 | |
| | | \|\|\|\|\|\|\|\|\|\|\|\|\|\|\|\|\|\| \|\|\|\|\|\|\|\|\| \|\|\|\|\|\|\|\|\|\|\| \|\|\|\|\|\|\|\|\|\|\|\|\|\|\| \|\|\| | | |
| K-18: | 947 | TGAGACGTACAGTCCAGTCCTTGGAGATCGACCTGGACTCCATGAGAAATCTGAAGGCCA | 1006 | |
| NOV4: | 951 | GCTTGGAGAACAGCCTGAGGGAGGTGGAGGCCCGCTACGCCCTGCAGATGGAGCAGCTCA | 1010 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\| | | |
| K-18: | 1007 | GCTTGGAGAACAGCCTGAGGGAGGTGGAGGCCCGCTACGCCCTACAGATGGAGCAGCTCA | 1066 | |
| NOV4: | 1011 | ACAGAATCCTGCTGTACTTGGAGTCAAAGCTGGCACAGAACTGGGCAGAGGGCCAGCGCA | 1070 | |
| | | \|\| \| \|\|\|\|\|\|\|\|\| \|\| \| \|\|\|\|\|\| \|\|\|\|\|\|\|\|\|\|\|\| \| \|\|\|\|\|\|\|\|\|\| \|\|\|\|\|\|\| | | |
| K-18: | 1067 | ACGGGATCCTGCTGCACCTTGAGTCAGAGCTGGCACAGACCCGGGCAGAGGGACAGCGCC | 1126 | |
| NOV4: | 1071 | AGGTCCAGGAGTACAAGGACTTGCTGAACATCAGGGTCAAGCTGGAGGCTGAGATCGCCA | 1130 | |
| | | \|\|\| \|\|\|\|\|\|\|\|\| \|\|\| \| \|\|\|\|\|\|\|\|\|\|\|\| \|\|\|\|\|\|\|\|\|\|\|\|\|\|\|\|\|\|\|\|\|\|\|\|\|\| | | |
| K-18: | 1127 | AGGCCCAGGAGTATGAGGCCCTGCTGAACATCAAGGTCAAGCTGGAGGCTGAGATCGCCA | 1186 | |
| NOV4: | 1131 | CCTACCGCCGCCTGCTGGAAGACAGCGAGGGCCTCAATCTTGGTGATGCCCTGGACAGCA | 1190 | |
| | | \|\|\|\|\|\|\|\|\|\|\|\|\|\|\|\|\|\|\|\|\|\| \|\|\|\|\|\| \| \| \|\|\|\|\|\|\|\|\|\|\|\|\|\|\| \|\|\|\|\|\|\|\|\| | | |
| K-18: | 1187 | CCTACCGCCGCCTGCTGGAAGATGGCGAGGACTTTAATCTTGGTGATGCCTTGGACAGCA | 1246 | |
| NOV4: | 1191 | GCAACTCCATGCAAACCATCCAAAAGACCACCACCCGCCAGATAGTGGATAGCAAAGTGG | 1250 | |
| | | \|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\| \|\|\|\|\|\|\|\|\| | | |
| K-18: | 1247 | GCAACTCCATGCAAACCATCCAAAAGACCACCACCCGCCGGATAGTGGATGGCAAAGTGG | 1306 | |
| NOV4: | 1251 | TGTCTGAGATCAGTGACACCAAAGTTCTGAGACATTAAGCCAGCAGAAG 1299 | | |
| | | \|\|\|\|\|\|\|\|\| \|\| \|\|\|\|\|\|\|\|\|\|\|\|\|\|\|\|\|\| \|\|\|\|\|\|\|\|\|\|\|\|\|\|\|\|\| | | |
| K-18: | 1307 | TGTCTGAGACCAATGACACCAAAGTTCTGAGGCATTAAGCCAGCAGAAG 1355 | | |

TABLE 14

| | | | | |
|---|---|---|---|---|
| NOV4: | 1 | MSFTTPSTFSTNYQSLGSVQPPSYGTWPVSSAASIYAGTGGLGSQISMSCSTSFWGGLGS | 60 | (SEQ ID NO.: 8) |
| | | \|\|\|\|\| \|\|\|\|\|\|\|+\|\|\|\|\|\| \|\|\|\| \|\|\|\|\|\|\|+\|\|\| \|\| \|\|+\|\|+\| \|\|\|\| \|\|+\|\| | | |
| HK18: | 1 | MSFTTRSTFSTNYRSLGSVQAPSYGARPVSSAASVYAGAGGSGSRISVSRSTSFRGGMGS | 60 | (SEQ ID NO.: 36) |
| NOV4: | 61 | GGLATEMAGGLAEMGGIQNEKETMQSLNDHL-DYLDRVRNLETENWRLESKIQEYLEKR- | 118 | |
| | | \|\|\|\|\| +\|\|\|\|\| \|\|\|\|\|\|\|\|\|\|\|\|\|\|\|\| \| \|\|\|\|\|\|+\|\|\|\|\| \|\|\|\|\|\|+\|+\|\|\|+ | | |
| HK18: | 61 | GGLATGIAGGLAGMGGIQNEKETMQSLNDRLASYLDRVRSLETENRRLESKIREHLEKKG | 120 | |
| NOV4: | 119 | PHVRDWGHYFKTIKELRAQIFANTVDNVHILQIDNARLAADDFRVKYETELAMRQSVES | 178 | |
| | | \| \|\|\|\| \|\|\|\| \|++\|\|\|\|\|\|\|\|\|\|\|\| \|+\|\|\|\|\|\|\|\|\|\|\|\|\|\|\|\|\|\|\|\|\|\|\|\|\|\|\|+ | | |
| HK18: | 121 | PHVRDWGHYFKTIKELRAQIFANTVDNVHILQIDNARLAADDFRVKYETELAMRQSVER | 180 | |
| NOV4: | 179 | NIHGLCKVIDDTNVTLLQLETMGALKEELLLMKIIHEEEVKGLQVQIANSGLAVEVDAP | 238 | |
| | | +\|\|\|\| \|\|\|\|\|\|\|+\| \|\|\|\|\|+ \|\|\|\|\|\|\| \|\|\|\|\|\|\|\|\|\|\|\|\| \|\|\|+\|\|\| \|\|\|\|\|\| | | |
| HK18: | 181 | DIHGLRXVIDDTNITRLQLETEIELKEELLFNXKNHEEEVKGLQAQIASSGLTVEVDAP | 240 | |
| NOV4: | 239 | KSQVLAXMADIRAQYDELSQKNSEKLGKYWSQQTEESTTVVTTHSAKVRAAEMT-TELR | 297 | |
| | | \|\|\| \|\|\|+\|\|\|\|\|\|\|\|\|\|++\|\| \|+\| \|\|\|\|\|\| \|\|\|\|\|\|\|\|\| \|\|+\| \|\|\| \| \|\|\|\| | | |
| HK18: | 241 | KSQDLAKIMADIRAQYDELARXNREELDKYWSQQIEESTTVVTTQSAEVGAAETTLTELR | 300 | |
| NOV4: | 298 | RTVQCLEIDLDSMRNLKTSLENSLREVEARYALQMEQLNRILLYLESKLAQNWAEGQRXV | 357 | |
| | | \|\|\|\| \|\|\|\|\|\|\|\|\|\|\|\| \|\|\|\|\|\|\|\|\|\|\|\|\|\|\|\|\|\|\|\|\| \|\|\|+\|\|\|+\|\|\| \|\|\|\|\|\|+ | | |
| HK18: | 301 | RTVQSLEIDLDSMRNLKASLENSLREVEARYALQMEQLNGILLHLESELAQTRAEGQRQA | 360 | |
| NOV4: | 358 | QEYKDLLNIRVKLEAEIATYRPLLEDSEGLNLGDALDSSNSMQTIQKTTTRQIVDSKVVS | 317 | |
| | | \|\|\|+ \|\|\|\|+\|\|\|\|\|\|\|\|\|\|\|\|\|\|\|\| \| \|\|\|\|\|\|\|\|\|\|\|\|\|\|\|\|\|\|\|\|\|+\|\|\| \|\|\|\| | | |
| HK18: | 361 | QEYEALLNIKVKLEAEIATYRRLLEDGEDFNLGDALDSSNSMQTIQKTTTRRIVDGKVVS | 420 | |
| NOV4: | 318 | EISDTKVLRH 328 | | |
| | | \| +\|\|\|\|\|\|\| | | |
| HK18: | 421 | ETNDTKVLRH 430 | | |

Where | indicates identity and + indicates similarity.

Intermediate filaments (IFs) are a structurally related family of cellular proteins that appear to be intimately involved with the cytoskeleton. The common structural motif shared by all IFs is a central alpha-helical ‘rod domain’ flanked by variable N- and C-terminal domains. The rod domain, the canonical feature of IFs, has been highly conserved during evolution. The variable terminals, however, have allowed the known IFs to be classified into 6 distinct types by virtue of their differing amino acid sequences (See Steinert and Roop, 1988, Annu. Rev. Biochem. 57:593). Keratins compose types I and II; intermediate filaments desmin, vimentin, GFAP, and peripherin, type III; neurofilaments, type IV, and nuclear lamins, type V. Nestin (600915) has been classed as type VI (See Lendahl et al., 1990, Cell 60:585).The acidic keratins are coded by genes KRT9 to KRT19. These genes are located on mouse chromosome 11 and human chromosome 17, except for KRT18 which may be located on human chromosome 12 (see later). The basic keratins are coded by genes KRT1 to KRT8, which are located on mouse chromosome 15 and human chromosome 12.

Ku and colleagues described transgenic mice that express point-mutant K18 and develop chronic hepatitis and hepatocyte fragility in association with disruption of hepatocyte keratin filaments. They showed that transgenic mice expressing mutant K18 are highly susceptible to hepatotoxicity after acute administration of acetaminophen or chronic ingestion of griseofulvin. The authors concluded that the predisposition to hepatotoxicity results directly from the keratin mutation since nontransgenic or transgenic mice that express normal human K18 are more resistant. Hepatotoxicity was manifested by a significant difference in lethality, liver histopathology, and biochemical serum testing. Keratin glycosylation decreased in all griseofulvin-fed mice, whereas keratin phosphorylation increased dramatically preferentially in mice expressing normal K18. The phosphorylation increase in normal K18 after griseofulvin feeding appeared to involve sites that are different from those that increased after partial hepatectomy. Ku and co-workers stated that this dramatic phosphorylation increase in nonmutant keratins could provide survival advantage to hepatocytes (See Ku et al., J. Cell Biol. 131:1305).

K8/18 is the major keratin pair in epithelia of the type found in liver, pancreas, and intestine. Transgenic mice that express mutant keratin 18, as already noted, develop chronic hepatitis, and have an increased susceptibility to drug-induced hepatotoxicity. By studying patients with liver disease of unknown cause for mutations in KRT18, Ku and colleagues described a his127leu (H127L) KRT mutation in a patient with cryptogenic cirrhosis that was germline transmitted. The mutant KRT18 isolated from the liver explant, or after expression in bacteria, showed an altered migration on 2-dimensional gel analysis as compared with normal human liver or bacterially expressed KRT18. Electron microscopy of in vitro assembled mutant KRT18 and wildtype KRT8 showed an assembly defect as compared with normal KRT8/18 assembly. The results suggested that mutations in KRT18 predispose to cryptogenic cirrhosis in humans (See Ku et al., 1997, J. Clin. Invest. 99:19).

NOV4 represents a new member of the cytokeratin-18 family. NOV4 is useful in determining changes in expression of genes contained within the cytokeratin-18 protein family. NOV4 satisfies a need in the art by providing new diagnostic or therapeutic compositions useful in the treatment of disorders associated with alterations in the expression of members of cytokeratin-18-like protein-associated proteins. NOV4 nucleic acids, polypeptides, antibodies, and other compositions of the present invention are useful in the treatment and/or diagnosis of a variety of diseases and pathologies, including by way of nonlimiting example, those involving hepatic disorders, e.g. cryptogenic cirrhosis.

NOV5

A NOV5 sequence according to the invention includes a nucleic acid sequence encoding a polypeptide related to the metallocarboxypeptidase family of proteins. A NOV5 nucleic acid maps to human chromosome 20. A NOV5 nucleic acid and its encoded polypeptide includes the sequences shown in Table 15. A NOV5 nucleic acid is likely to be expressed in testis, spleen, salivary gland, brain, heart, thyroid, bone marrow, lung, kidney, uterus, ovary and germ cells. The disclosed nucleic acid (SEQ ID NO:9) is 2,202 nucleotides in length and contains an open reading frame (ORF) that begins with an ATG initiation codon at nucleotides 1-3 and ends with a TGA stop codon at nucleotides 2,200-2,200. The representative ORF encodes a 733 amino acid polypeptide (SEQ ID NO:10) with a predicted molecular weight of 81,573.8 Da. PSORT analysis of a NOV5 polypeptide predicts a lysosomal localization with a certainty of 0.5487 and a secreted protein with a certainty of 0.5469. SIGNALP analysis suggests the presence of a signal peptide, with the most likely cleavage site between position 20 and 21 of SEQ ID NO.: 10).

TABLE 15

| | |
|---|---|
| ATGTGGGGGCTCCTGCTCGCCCTGGCCGGCTCGCGCCGGCCGTCGGCCCGGCTCTG | (SEQ ID NO.: 9) |
| GGGGCGCCCAGGAACTCGGTGCTGGGCCTCGCGCAGCCCGGGACCACCAAGGTCCC | |
| AGGCTCGACCCCGGCCCTGCATAGCAGCCCGGCACAGCCGTCGGCGGAGACAGCTA | |
| ACACCTCAGAACAGCATGTCCGGATTCGAGTCATCAAGAAGAAAAAGGTCATTATG | |
| AAGAAGCGGAAGAAGCTAACTCTAACTCGCCCCACCCCACTGGTGACTGCCGGGCC | |
| CCTTGTGACCCCCACTCCAGCAGGGACCCTCGACCCCGCTGAGAAACAAGAACCAG | |
| GCTGTCCTCCTTTGGGTCTGGAGTCCCTGCGAGTTCAGATAGCCGGCTTGAGGCAT | |
| CCAGCAGCCAGTCCTTTTGGTCTTGGACCACACCGAGGACGGCTCAACATTCAGTCAG | |
| GCCTGGAGGACGGCGATCTATATGATGGAGCCTGGTGTGCTGAGGAGCAGGACGCC | |
| GATCCATGGTTTCAGGTGGACGCTGGGCACCCCACCCGCTTCTCGGGTGTTATCACA | |
| CAGGGCAGGAACTCTGTCTGGAGGTATGACTGGGTCACATCATACAAGGTCCAGTTC | |
| AGCAATGACAGTCGGACCTGGTGGGGAAGTAGGAACCACAGCAGTGGGATGGACGC | |
| AGTGTTTCCTGCCAATTCAGACCCAGAAACTCCAGTGCTGAACCTCCTGCCGGAGCC | |
| CCAGGTGGCCCGCTTCATTCGCCTGCTGCCCCAGACCTGGCTCCAGGGAGGCGCGCC | |
| TTGCCTCCGGGCAGAGATCCTGGCCTGCCCAGTCTCAGACCCCAATGACCTATTCCT | |
| TGAGGCCCCTGCGTCGGGATCCTCTGACCCTCTAGACTTTCAGCATCACAATTACAA | |
| GGCCATGAGGAAGCTGATGAAGCAGGTACAAGAGCAATGCCCCAACATTACCTCGC | |
| ATCTACAGCATTGGGAAGAGCTACCAGGGCCTGAAGCTGTATGTGATGGAAATGTC | |
| GGACAAGCCTGGGGAGCATGAGCTGGGTGAGCCTGAGGTGCGCTACGTGGCTGGCA | |
| TGCATGGGAACGAGGCCCTGGGGCGGGAGTTGCTTCTGCTCCTGATGCAGTTCCTGT | |
| GCCATGAGTTCCTGCGAGGGAACCCACGGGTGACCCGGCTGCTCTCTGAGATGCGC | |
| ATTCACCTGCTGCCCTCCATGAACCCTGATGGCTATGAGATCGCCTACCACCGGGGT | |
| TCAGAGCTGGTGGGCTGGGCCGAGGGCCGCTGGAACAACCAGAGCATCGATCTTAA | |
| CCATAATTTTGCTGACCTCAACACACCACTGTGGGAAGCACAGGACGATGGGAAGG | |
| TGCCCCACATCGTCCCCAACCATCACCTGCCATTGCCCACTTACTACACCCTGCCCA | |
| ATGCCACCGTGGCTCCTGAAACGCGGGCAGTAATCAAGTGGATGAAGCGGATCCCC | |
| TTTGTGCTAAGTGCCAACCTCCACGGGGGTGAGCTCGTGGTGTCCTACCCATTCGAC | |
| ATGACTCGCACCCCGTGGGCTGCCCGCGAGCTCACGCCCACACCAGATGATGCTGTG | |
| TTTCGCTGGCTCAGCACTGTCTATGCTGGCAGTAATCTGGCCATGCAGGACACCAGC | |
| CGCCGACCCTGCCACAGCCAGGACTTCTCCGTGCACGGCAACATCATCAACGGGGC | |
| TGACTGGCACACGGTCCCCGGGAGTATGAATGACTTCAGCTACCTACACACCAACTG | |
| CTTTGAGGTCACTGTGGAGCTGTCCTGTGACAAGTTCCCTCACGAGAATGAATTGCC | |
| CCAGGAGTGGGAGAACAACAAAGACGCCCTCCTCACCTACCTGGAGCAGGTGCGCA | |
| TGGGCATTGCAGGAGTGGTGAGGGACAAGGACACGGAGCTTGGGATTGCTGACGCT | |
| GTCATTGCCGTGGATGGGATTAACCATGACGTGACCACGGCGTGGGGCGGGGAATTA | |
| TTGGCGTCTGCTGACCCCAGGGGACTACATGGTGACTGCCAGTGCCGAGGGCTACCA | |
| TTCAGTGACACGGAACTGTCGGGTCACTTTGAAGAGGGGCCCCTTCCCCTGCAATTT | |
| CGTGCTCACCAAGACTCCCAAACAGAGGCTGCGCGAGCTGCTGGCAGCTGGGGCCA | |
| AGGTGCCCCCGGACCTTCGCAGGCGCCTGGAGCGGCTAAGGGGACAGAAGGATTGA | |
| MWGLLLALAGFAPAVGPALGAPRNSVLGLAQPGTTKVPGSTPALHSSPAQPSAETANTA | (SEQ ID NO.: 10) |
| EQHVRIRVIKKKKVIMKKRKKLTLTRPTPLVTAGPLVTPTPAGTLDPAEKQEPGCPPLGLE | |
| SLRVSDSRLEASSSQSFGLGPHRGRLNIQSGLEDGDLYDGAWCAEEQDADPWFQVDAG | |
| HPTRFSGVITQGRNSVWRYDWVTSYKVQFSNDSRTWWGSRNHSSGMDAVFPMSTSDPET | |
| PVLNLLPEPQVARFIRLLPQTWLQGGAPCLRAELACPVSDPNDLFLEAPASGSSDPLDFQ | |
| HIHNYKAMRKLMKQVQEQCPNITRIYSIGKSYQGLKLYVMEMSDKPGEHELGEPEVRYV | |
| AGMHGNEALGRELLLLLMQFLCHEFLRGNPRVTRLLSEMRIHLLPSMNODGYEIAYHRG | |
| SELVGWAEGRWNNQSLDLNHNFADLNTPLWEAQDDGKVPHIVPNHHLPLPTYYTLPNA | |
| TVAPETRAVIKWMKRIPFVISANLHGGELVVSYPFDMTRTPWAARELTPTPDDAVFRWL | |
| STVYAGSNLAMQDTSRRPCHSQDFSVHGNIJNGADWHTVPGSMNDFSYLHTNCFEVTVE | |
| LSCKDFPHENELPQEWENNKDALLTYLEQVRMGIAGVVRDKDTELGIADAVIAVDGINH | |
| DVTTAWGGDYWRLLTPGDYMVTASAEGYHSVTRNCRVTLKIRGPFPCNFVLTKTPKQR | |
| LRELLAAGAKVPPDLRRRLERLRGQKD | |

A NOV5 polypeptide has homology (84% identity, 89% similarity) with a mouse metallocarboxypeptidase CPX-1 polypeptide (CPX1; EMBL Accession No.: Q9Z100), as is shown in Table 16. Also, a NOV5 polypeptide has a high degree of homology with an uncharacterized human protein APG04 (AGP04; PatP Accession No.: B36174), as is shown in Table 17.

TABLE 16

| | | | | |
|---|---|---|---|---|
| NOV5: | 1 | MWGLLLALAGFAPAVGPALGAPRNSVLGLAQPGTTKVPGSTPALHSSPAQPSAETANTSE | 60 | (SEQ ID NO.: 10) |
| | | \|\|\|\|\|\|\|+ \|\|\|+\|\| \|\|\|\| \|\| \|\|\| \|\|\|\| \| \|\|\| \|\|\|\| + \|\|\| | | |
| CPX1: | 1 | MWGLLLAVTAFAPSVGLGLGAPSASVPGLA-------PGSTLAPHSSVAQPSTKANETSE | 53 | (SEQ ID NO.: 37) |
| NOV5: | 61 | QHVRIRVIKKKKVIMKKRKKLTLTRPTPLVTAGPLVTPTPAGTLDPAEKQEPGCPPLGLE | 120 | |
| | | +\|\|\|+\|\|\|\|\|\|\|+++\|\|\|\|\|\| \| \|\| \|\| \|+\| \|\| \|\| \|\|\|\|\|\|\|\|\|\|\|\|\| | | |
| CPX1: | 54 | RHVRLRVIKKKKIVVKKRKKLR--HPGPLGTARPVVPTHPAKTLTLPEKQEPGCPPLGLE | 111 | |
| NOV5: | 121 | SLRVSDSRLEASSSQSFGLGPHRGRLNIQSGLEDGDLYDGAWCAEEQDADPWFQVDACHP | 180 | |
| | | \|\|\|\|\|\|\|+\|\|\|\|\|\|\|\|\|\|\|\| \|\|\|\|\|\|\|\|\|\|\|\|\|\|\|\|\|\|\|\|\|\|\|\|+\|\| +\|\| \|\|\|\| +\| | | |
| CPX1: | 112 | SLRVSDSQLEASSSQSFGLGAHRGRLNIQSGLEDGDLYDGAWCAEQQDTEPWLQVDAKNP | 171 | |
| NOV5: | 181 | TRFSGVITQGRNSVWRYDWVTSYKVQFSNDSRTWWGSRNHSSGMDAVFPANSDPETPVLN | 240 | |
| | | \|\|+\|++\|\|\|\|\|\|\|\|\|\|\|\|\|\|\|+\|\|\|\|\|\|\|\|+\|\|\| \|\|\| \|+\|\|\| \|\|\|\|\|\|\| \|\|\|\|\|\| | | |
| CPX1: | 172 | VRFAGIVTQGRNSVWRYDWVTSFKVQFSNDSQTWWKSRN-STGMDIVFPANSDAETPVLN | 230 | |
| NOV5: | 241 | LLPEPQVARFIRLLPQTWLQGCAPCLRAEILACPVSDPNNLFLEAPASGSSDPLDFQHHN | 300 | |
| | | \|\|\|\|\|\|\|\|\|\|\|\|\|\|\|\|\|\| \|\|\| \|\|\|\|\|\|\|\|\|\|\|\|\|\|\|\|\|\|\| \|\| \|\|\|+ \|\|\|+\|\|\| | | |
| CPX1: | 231 | LLPEPQVARFIRLLPQTWFQGGVPCLRAEILACPVSDPNDLFLPEAHTLGNNSLDFRHHN | 290 | |
| NOV5: | 301 | YKAMRKLMKQVQEQCPNITRIYSTCKSYQGLKLYVMEMSDKPGEHELGEPEVRYVAGMHG | 360 | |
| | | \|\|\|\|\|\|\|\|\|\|\| \|\|\|\|\|\|\|\|\|\|\|\|\|\|\|+\|\|\|\|\|\|\|\|\|\|\|\| \|\|\|\|\|\|\|\|\|\|\|\|\|\|\|\|\|\|\| | | |
| CPX1: | 291 | YKAMRKLMKQVNEQCPNITRIYSIGKSHQGLKLYVMEMSDHPGEHELGEPEVRYVAGMHG | 350 | |
| NOV5: | 361 | NEALGRELLLLLMQFLCHEFLRGNPRVTRLLSEMRIHLLPSMNPDGYEIAYHRGSELVGW | 420 | |
| | | \|\|\|\|\|\|\|\|\|\|\|\|\|\|\|\|\|\|\|\|\|\|\|+\|\|\|\|\|\|\|+\| \|\|\|\|\|\|\|\|\|\|\|\|\|\| \|\|\|\|\|\|\|\|\|\|\| | | |
| CPX1: | 351 | NEALGRELLLLLMQFLCHEFLRGDPRVTRLLTETRIHLLPSMNPDGYETAYHRGSELVGW | 410 | |
| NOV5: | 421 | AEGRWNNQSIDLNHNFADLNTPLWEAQDDGKVPHIVPNIHLPLPTYYTLPNATVAPETRA | 480 | |
| | | \|\|\|\|\| +\| \|\|\|\|\|\|\|\|\|\|\|\| \|\| \|+\|\|\| \|\| \|\|\|\|\|\|\|\|\|\|\|\|\|\|\|\|\|\|\|\|\|\|\| \| | | |
| CPX1: | 411 | AEGRWTHQGIDLNHNFADLNTQLWYAEDDGLVPDTVPNHHLPLPTYYTLPNATVAPETWA | 470 | |
| NOV5: | 481 | VIKWMKRIPFVLSANLHGGELVVSYPFDMTRTPWAARELTPTPDDAVFRWLSTVYAGSNL | 540 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|+\| | | |
| CPX1: | 471 | VIKWMKRIPFVLSANLHGGELVVSYPFDMTRTPWAARELTPTPDDAVFRWLSTVYAGTNR | 530 | |
| NOV5: | 541 | AMQDTSRRPCHSQDFSVHGNIINCADWNTVPGSMNDFSYLHTNCFEVTVELSCDKFPHEN | 600 | |
| | | \|\|\|\|\| \|\|\|\|\|\|\|\|\|\|+\|\|\|+\|\|\|\|\|\|\|\|\|\|\|\|\|\|\|\|\|\|\|\|\|\|\|\|\|\|\|\|\|\|\|\|\|\|\|\|\|\| | | |
| CPX1: | 531 | AMQDTDRRPCHSQDFSLHGNVINGADWHTVPGSMNDFSYLHTNCFEVTVELSCDKFPHEK | 590 | |
| NOV5: | 601 | ELPQEWENNKDALLTYLEQVRMGIAGVVRDKDTELGIADAVIAVDGINHDVTTAWGGDYW | 660 | |
| | | \|\|\|\|\|\|\|\|\|\|\|\|\|\|\|\|\|\|\|\|\|\|\|\| \|\|\|\|\|\|\|\|\|\|\|\|\|\|\|\|\|\|\|+\|\|\|\|\|\|\|\|\|\|\|\|\|\|\| | | |
| CPX1: | 591 | ELPQEWENNKDALLTYLEQVRMGITGVVRDKDTELGIADAVIAVEGINHDVTTAWGGDYW | 650 | |
| NOV5: | 661 | RLLTPGDYMVTASAEGYHSVTRNCRVTLKRGPFPCNFVLTKTPKQRLRELLAAGAKVPPD | 720 | |
| | | \|\|\|\|\|\|\|\|+\|\|\|\|\|\|\|\|\|+\| ++\|+\|\| + \|\| \|\|\|\|+\|\|\|\|\|\|+\|\|\|\|\|\|\| \|+\|\|\| | | |
| CPX1: | 651 | RLLTPGDYVVTASAEGYHTVRQHCQVTFEEGPVPCNFLLTKTPKERLRELLATRGKLPPD | 710 | |
| NOV5: | 721 | LRRRLERLRGQKD 733 | | |
| | | \|\|\|+\|\|\|\|\|\|\|\| | | |
| CPX1: | 711 | LRRKLERLRGQK 722 | | |

Where | indicates identity and + indicates similarity.

TABLE 17

| | | | | |
|---|---|---|---|---|
| NOV5: | 1 | MWGLLLALAGFAPAVGPALGAPRNSVLGLAQPGTTKVPGSTPALHSSPAQPSAETAN-TS | 59 | (SEQ ID NO.: 10) |
| | | \|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\| \|\| | | |
| ACP04: | 1 | MWGLLLALAAFAPAVGPALGAPRNSVLGLAQPGTTKVPGSTPALHSSPAQPPAETANGTS | 60 | (SEQ ID NO.: 38) |
| NOV5: | 60 | EQHVRIRVIKKKKVIMKKRKKLTLTRPTPLVTACPLVTPTPAGTLDPAEKQEPGCPPLGL | 119 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\| | | |
| AGP04: | 61 | EQHVRIRVIKKKKVIMKKRKKLTLTRPTPLVTAGPLVTPTPAGTLDPAEKQETGCPPLGL | 120 | |
| NOV5: | 120 | ESLRVSDSRLEASSSQSFGLGPHRGRLNIQSGLEDGDLYDGAWCAEEQDADPWFQVDAGH | 179 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| AGP04: | 121 | ESLRVSDSRLEASSSQSFGLGPHRGRLNIQSCLEDGDLYDGAWCAEEQDADPWFQVDAGH | 180 | |
| NOV5: | 180 | PTRFSGVITQGRNSVWRYDWVTSYKVQFSNDSRTWWGSRNHSSGMDAVFPANSDPETPVL | 239 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| AGP04: | 181 | PTRFSGVITQGRNSVWRYDWVTSYKVQFSNDSRTWWCSRNHSSGMDAVFPANSDPETPVL | 240 | |
| N0V5: | 240 | NLLPEPQVARFIRLLPTTWLQGGAPCLRAETLACPVSDPNDLFLEAPASGSSDPLDFQHH | 299 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| AGP04: | 241 | NLLPEPQVARFIRLLPQTWLQGGAPCLRAEILACPVSDPNDLFLEAPASGSSDPLDFQHH | 300 | |
| NOV5: | 300 | NYKAMRKLMKQVQEQCPNITRIYSIGKSYQGLKLYVMEMSDKPGEHELGEPEVRYVAGMH | 359 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| AGP04: | 301 | NYKAMRKLMKQVQEQCPNITRIYSIGKSYQGLKLYVMEMSDKPGEHELGEPEVRYVAGMH | 360 | |
| NOV5: | 360 | GNEALGRELLLLLMQFLCHEFLRGNPRVTRLLSEMRIHLLPSMNPDGYEIAYHRGSELVG | 419 | |
| | | \|\|\|\|\|\|\|\|\|\|\|\|\|\|\|\|\|\|\|\|\|\|\|\|\|\|+\|\|\|\|\|\|\|\|\|\|\|\|\|\|\|\|\|\|\|\|\|\|\|\|\|\|\|\|\|\|\|\|\| | | |
| AGP04: | 361 | GNEALGRELLLLLMQFLCHEFLRGNPQVTRLLSEMRIHLLPSMNPDGYEIAYHRGSELVG | 420 | |
| NOV5: | 420 | WAEGRWNNQSIDLNHNFADLNTPLWEAQDDGKVPHIVPNHHLPLPTYYTLPNATVAPETR | 479 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| AGP04: | 421 | WAEGRWNNQSIDLNHNFADLNTPLWEAQDDGKVPHIVPNHHLPLPTYYTLPNATVAPETR | 480 | |
| N0V5: | 480 | AVIKWMKRIPFVLSANLHGGELVVSYPFDMTRTPWAARELTPTPDDAVFRWLSTVYAGSN | 539 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| AGP04: | 481 | AVIKWMKRIPFVLSANLHGGELVVSYPFDMTRTPWAARELTPTPDDAVFRWLSTVYAGSN | 540 | |
| NOV5: | 540 | LAMQDTSRRPCHSQDFSVHGNIINGADWHTVPGSMNDFSYLHTNCFEVTVELSCDKFPHE | 599 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| AGP04: | 541 | LAMQDTSRRPCHSQDFSVHGNIINGADWHTVPGSNNDFSYLHTNCFEVTVELSCDKFPHE | 600 | |
| NOV5: | 600 | NELPQEWENNXDALLTYLEQVRMCIAGVVRDKDTELGIADAVIAVDGINHDVTTAWGGDY | 659 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| AGP04: | 601 | NELPQEWENNKDALLTYLEQVRMGIAGVVRDKDTELGIADAVIAVDGINHDVTTAWGGDY | 660 | |
| NOV5: | 660 | WRLLTPCDYMVTASAEGYHSVTRNCRVTLKRGPFPCNFVLTKTPKQRLRELLAAGAKVPP | 719 | |
| | | \|\|\|\|\|\|\|\|\|\|\|\|\|\|\|\|\|\|\|\|\|\|\|\|\|\|\|\| + \|\|\|\|\|\|\|\|\|\|\|\|\|\|\|\|\|\|\|\|\|\|\|\|\|\|\|\|\| | | |
| AGP04: | 661 | WRLLTPGDYMVTASAEGYHSVTRNCRVTFEEGPFPCNFVLTKTPKQRLRELLAAGAKVPP | 720 | |
| NOV5: | 720 | DLRRRLERLRGQKD 733 | | |
| | | \|\|\|\|\|\|\|\|\|\|\|\|\|\| | | |
| AGP04: | 721 | DLRRRLERLRGQKD 734 | | |

Where | indicates identity and + indicates similarity.

Metallocarboxypeptidases are members of a gene family with broad gene expression patterns and in vivo functions. The recent finding that Cpe(fat)/Cpe(fat) mice, which lack carboxypeptidase E (CPE) activity because of a point mutation, are still capable of a reduced amount of neuroendocrine peptide processing suggested that additional carboxypeptidases (CPs) participate in this processing reaction. Searches for novel members of the CPE gene family led to the discovery of CPD, CPZ, AEBP1, and CPX-2. Like AEBP1 and CPX-2, CPX-1 contains an N-terminal region of 160 amino acids with sequence similarity to the discoidin domain of a variety of proteins. The 410-residue CP-like domain of CPX-1 has 54% to 62% amino acid sequence identity with AEBP1 and CPX-2 and 33% to 49% amino acid identity with other members of the CPE subfamily. However, several active-site residues that are important for catalytic activity of other CPs are not conserved in CPX-1. Furthermore, CPX-1 expressed in either the baculovirus system or the mouse AtT-20 cell line does not cleave standard CP substrates. Northern blot analysis shows the highest levels of CPX-1 mRNA in testis and spleen and lower levels in salivary gland, brain, heart, lung, and kidney. In situ hybridization of CPX-1 mRNA in embryonic and fetal mouse tissue showed expression throughout the head and thorax, with abundance in primordial cartilage and skeletal structures. In the head, high levels of CPX-1 mRNA are associated with the nasal mesenchyme, primordial cartilage structures in the ear, and the meninges. In the thorax, CPX-1 mRNA is expressed in multiple developing skeletal structures, including chondrocytes and perichondrial cells of the rib, vertebral, and long-bone primordia. CPX-1 may have a role in development, possibly mediating cell interactions via its discoidin domain. (See Lei et al., 1999, DNA Cell Biology 18:175).

NOV5 represents a new member of the metallocarboxypeptidase family of proteins. NOV5 is useful in determining changes in expression of genes contained within the metallocarboxypeptidase protein family. NOV5 will be useful in identifying testis, spleen, salivary gland, brain, heart, thyroid, bone marrow, lung, kidney, uterus, ovary tissue and germ cells. NOV5 satisfies a need in the art by providing new diagnostic or therapeutic compositions useful in the treatment of disorders associated with alterations in the expression of members of the metallocarboxypeptidase-associated protein family of proteins. NOV5 nucleic acids, polypeptides, antibodies, and other compositions of the present invention are useful in the treatment and/or diagnosis of a variety of diseases and pathologies, including by way of nonlimiting example, those involving metabolic disorders of the pancreas, e.g. acute pancreatitis.

NOV6

A NOV6 sequence according to the invention includes a nucleic acid sequence encoding a polypeptide related to the mast cell protease-6 family of proteins. A NOV6 nucleic acid and its encoded polypeptide includes the sequences shown in Table 18. The disclosed nucleic acid (SEQ ID NO: 11) is 846 nucleotides in length and contains an open reading frame (ORF) that begins with an ATG initiation codon at nucleotides 6-8 and ends with a TGA stop codon at nucleotides 840-842. The representative ORF encodes a 278 amino acid polypeptide (SEQ ID NO:12) with a predicted molecular weight of 30,570.1 Da. PSORT analysis of a NOV6 polypeptide predicts a lysosomal localization with a certainty of 0.8650. SIGNALP analysis suggests the presence of a signal peptide, with the most likely cleavage site between position 17 and 18 of SEQ ID NO.: 12). Putative untranslated regions upstream and downstream of the open reading frame are underlined in SEQ ID NO.: 11.

TABLE 18

| | |
|---|---|
| CGCAGATGCTGTGGCTGCTATTCCTGACCCTCCCCTGCCTGGGGGGCTCCATGTCCA | (SEQ ID NO.: 11) |
| AGACCCCAGTGCCCGTCCCAGAGAATGACCTGGTGGGCATTGTGGGGGGCCACAAT | |
| GCCCCCCCGGGGAAGTGGCCGTGGCAGGTCAGCCTGAGGGTCTACAGCTACCACTG | |
| GGCCTCCTGGGCGCACATCTGTGGGGGCTCCCTCATCCACCCCCAGTGGGTGCTGAc | |
| TGCTGCCCACTGCATITTCTGGAAGGACACCGACCCGTCCATCTACCGGATCCACGC | |
| TGGGGACGTGTATCTCTACGGGGGCCGGGGGCTGCTGAACGTCAGCCGGATCATCG | |
| TCCACCCCAACTATGTCACTGCGGGGCTGGGTGCGGATGTGGCCCTGCTCCAGCTGG | |
| TGAGCCCCATGATCGGAGCCGCTAATGTCAGGACGGTCAAGCTCTCCCCGGTCTCGC | |
| TGGAGCTCACCCCGAAGGACCAGTGCTGGGTGACTGGCTGGGGAGCGATCAGGATG | |
| TTCGAGTCGCTGCCGCCGCCCTACCGCCTGCAGCAGGCGAGTGTGCAGGTGCTGGAG | |
| AACGCCGTCTGTGAGCAGCCCTACCGCAACGCCTCAGGGCACACTGGCGACCGGCA | |
| GCTCATCCTGGATGACATGCTGTGTGCCGGCAGCGAGGGCCGAGACTCCTGTCAGG | |
| GTGACTCCGGCGGCCCTCTGGTCTGCAGGCTGCGGGGGTCCTGGCGCCTGGTGGGGG | |
| TGGTCAGCTGGGGCTACGGCTGTACCCTGCGGGACTLTCCCGGCGTCTACACCCACG | |
| TCCAGATCTACGTGCTCTGGATCCTGCAGCAAGTCGGGGAGTTGCCCTGAGCAG | |
| MLWLLFLTLPCLGGSMSKTPVPVPENDLVGIVGGHNAPPGKWPWQVSLRVYSYHWAS | (SEQ ID NO.: 12) |
| WAHICGGSLLHPQWVLTAAHCLFWKDTDPSIYRIHAGDVYLYGGRGLLNVSRIIVIIPNYV | |
| TAGLGADVALLQLVSPMIGAANVRTVKLSPVSLELTPKDQCWVTGWGALRMFESLPPPY | |
| RLQQASVQVLENAVCEQPYRNASGHTGDRQULDDMLCAGSEGRDSCQGDSGGPLVCR | |
| LRGSWRLVGVVSWGYGCTLRDFPGVYThVQIYVLWILQQVGELP | |

A NOV6 nucleic acid has homology (99% identity) with an uncharacterized region of human chromosome 16 including clone LA16-303A1 (CHR16; GenBank Accession No.: HS303A1), as is shown in table 19. A NOV6 polypeptide has homology (51% identity, 89% similarity) with a mouse mast cell protease-6 precursor polypeptide (MCP6; SwissProt Accession No.: P21845), as is shown in Table 20. Also, a NOV6 polypeptide has homology with a human beta-tryptase precursor polypeptide (HBTP; SwissProt Accession No.: P20231), as is shown in Table 21. Expression profiling of a NOV6 nucleic acid is described in Example 4.

TABLE 19

| | | | | |
|---|---|---|---|---|
| NOV6: | 247 | ggaaggacaccgacccgtccatctaccggatccacgctggggacgtgtatctctacgggg | 306 | (SEQ ID NO.: 39) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| CHR16: | 21749 | ggaaggacaccgacccgtccatctaccggatccacgctggggacgtgtatctctacgggg | 21690 | (SEQ ID NO.: 40) |
| NOV6: | 307 | gccgggggctgctgaacgtcagccggatcatcgtccaccccaactatgtcactgcggggc | 366 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| CHR16: | 21689 | gccgggggctgctgaacgtcagccggatcatcgtccaccccaactatgtcactgcggggc | 21630 | |
| NOV6: | 367 | tgggtgcggatgtggccctgctccagctggtgagccccatgatcggagccgctaatgtca | 426 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\| | | |
| CHR16: | 21629 | tgggtgcggatgtggccctgctccagctggtgagccccatgatctgagccgctaatgtca | 21570 | |
| NOV6: | 427 | ggacggtcaagctctccccggtctcgctggagctcaccccgaaggaccagtgctgggtga | 486 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| CHR16:2 | 1569 | ggacggtcaagctctccccggtctcgctggagctcaccccgaaggaccagtgctgggtga | 21510 | |
| NOV6: | 487 | ctggctggggagcgatcaggatgttcg 513 | | |
| | | \|\|\|\|\|\|\|\|\|\|\|\|\|\|\|\|\|\|\|\|\|\|\|\|\|\|\| | | |
| CHR16: | 21509 | ctggctggggagcgatcaggatgttcg 21483 | | |

TABLE 20

| | | | | |
|---|---|---|---|---|
| NOV6: | 69 | PVPENDLVGIVGGHNAPPGKWPWQVSLRVYSYHWASWAHICGGSLIHPQWLTAAHCIFW | 248 | (SEQ ID NO.: 41) |
| | | \| \| \| \|\|\|\|\|\|\| \| \|\|\|\|\|\|\|\|\| + ++ \| \| \|\|\|\|\|\|\|\|\|\|\|\|\|\|\|\|+ | | |
| MCP6: | 23 | PRPANQRVGIVGGHEASESKWPWQVSLR-FKLNY--WIHFCGGSLIHPQWVLTAAHCVGP | 79 | (SEQ ID NO.: 42) |
| NOV6: | 249 | KDTDPSIYRIHAGDVYLYGGRGLLNVSRIIVHPNYVTAGLGADVALLQLVSPMIGAANVR | 428 | |
| | | \| ++\|+ + \|\|\| \| \|\|+++\|\|+\|\|\|+\| \|\| \|\|\|\|\|\|\|+\| \|+ + ++ | | |
| MCP6: | 80 | HIKSPQLFRVQLREQYLYYGDQLLSLNRIVVHPHYYTAEGGADVALLELEVPVNVSTHIH | 139 | |
| NOV6: | 429 | TVKLSPVSLELTPKDQCWVTGWGAIRMFESLPPPYRLQQASVQVLENAVCEQPYRNASGH | 608 | |
| | | + \| \| \| \| \|\|\|\|\|\|\| \| \| \|\|\|\|\| \|+\| \| ++\|\|++\|++ \| + | | |
| MCP6: | 140 | PISLPPASETFPPGTSCWVTGWGDIDNDEPLPPPYPLKQVKVPIVENSLCDRKYHTGL-Y | 198 | |
| NOVG: | 609 | TGDR-QLILDDMLCAGSEGRDSCQGDSGGPLVCRLRGSWRLVGVVSWGYGCTLRDFPGVY | 785 | |
| | | \|\|\| ++ \| \|\|\|\|\|+ \|\|\|\|\|\|\|\|\|\|\|\|\|\|+++\|+\| \|\|\|\|\|\| \|\| + \|\|+\| | | |
| MCP6: | 199 | TGDDFPIVHDGMLCAGNTRPDSCQGDSGGPLVCKVKGTNLQAGVVSWGEGCAQPNKPGIY | 258 | |
| NOV6: | 786 | THVQIYVLWILQQVGE 833 | | |
| | | \| \| \|+ \|\| + \| \| | | |
| MCP6: | 259 | TRVTYYLDWIKRYVPE 274 | | |

Where | indicates identity and + indicates similarity.

TABLE 21

| | | | | |
|---|---|---|---|---|
| NOV6: | 1 | MLWLLFLTLPCLGGSMSKTPVPVPENDLVGIVGGHNAPPGKWPWQVSLRVYSYHWASWAH | 60 | (SEQ ID NO.: 43) |
| | | \|\| \|\| \| \|\| \| \| \| \|\|\|\|\|\| \| \|\|\|\|\|\|\|\|\|\| \| \| \| | | |
| HBTP: | 1 | MLNLLLLALPVLASRAYAAPAPGQALQRVGIVGGQEAPRSKWPWQVSLRV---HGPYWMH | 57 | (SEQ ID NO.: 44) |
| NOV6: | 61 | ICGGSLIHPQWVLTAAHCIFWKDTDPSIYRIHAGDVYLYGGRGLLNVSRIIVHPNYVTAG | 120 | |
| | | \|\|\|\|\|\|\|\|\|\|\|\|\|\|\|\|\|+ \| + \|+ + +\|\| \|\| \|\|\|\|\|\|\|\| + \|\| | | |
| HBTP: | 58 | FCGGSLIHPQWVLTAAHCVCPDVKDLAALRVQLREQHLYYQDQLLPVSRIIVHPQFYTAQ | 117 | |
| NOV6: | 121 | LGADVALLQLVSPMIGAANVRTVXLSPVSLELTPKDQCWVTGWGAIRMFESLPPPYRLQQ | 180 | |
| | | +\|\|\|+\|\|\|+\| \|+ +++\| \|\| \| \| \| \| \|\|\|\|\|\|\| + \| \|\|\|\|+ \|+\| | | |
| HBTP: | 118 | IGADIALLELEEPVKVSSHVHTVTLPPASETFPPGMPCWVTGWGDVDNDERLPPPFPLKQ | 177 | |
| NOV6: | 546 | ASVQVLENAVCEQPYRNASGHTGDR-QLILDDMLCAGSEGRDSCQGDSGGPLVCRLRGSW | 722 | |
| | | \| ++\|\| +\|+ \| + +\|\|\| +++ \|\|\|\|\|\|\|\|+ \|\|\|\|\|\|\|\|\|\|\|\|\|\|++ \|+\| | | |
| HBTP: | 178 | VKVPIMENHICDAKY-HLGAYTGDDVRIVRDDNLCAGNTRDRSCQGDSGGPLVCKVNGTW | 236 | |
| NOV6: | 181 | RLVGVVSWGYGCTLRDFPGVYTHVQIYVLWILQQVGELP 220 | | |
| | | \|\|\|\|\|\| \|\| + \|\|+\|\| \| \|+ \|\| \| + \| | | |
| HBTP: | 237 | LQAGVVSWGEGCAQPNRPGIYTRVTYYLDWIHHYVPKKP 275 | | |

Where | indicates identity and + indicates similarity.

The term mastocytosis denotes a heterogenous group of disorders characterized by abnormal growth and accumulation of mast cells in one or more organs. Cutaneous and systemic variants of the disease have been described. Mast cell disorders have also been categorized according to other aspects, such as family history, age, course of disease, or presence of a concomitant myeloid neoplasm. However, so far, generally accepted disease criteria are missing. Recently, a number of diagnostic (disease-related) markers have been identified in mastocytosis research. These include the mast cell enzyme tryptase, CD2, and mast cell growth factor receptor c-kit (CD117). The mast cell enzyme tryptase is increasingly used as a serum- and immunohistochemical marker to estimate the actual spread of disease (burden of neoplastic mast cells). The clinical significance of novel mastocytosis markers is currently under investigation. First results indicate that they may be useful to define reliable criteria for the delineation of the disease.

NOV6 represents a new member of the mast cell protease-6 family of proteins. NOV6 is useful in determining changes in expression of genes contained within the mast cell protease-6 protein family. NOV6 satisfies a need in the art by providing new diagnostic or therapeutic compositions useful in the treatment of disorders associated with alterations in the expression of members of the mast cell protease-6-associated protein family of proteins. NOV6 nucleic acids, polypeptides, antibodies, and other compositions of the present invention are useful in potential therapeutic applications implicated in disorders characterized by abnormal growth and accumulation of mast cells in one or more organs including, but not limited to skin, ear and brain as well as other pathologies and disorder such as hemophilia, idiopathic thrombocytopenic purpura, autoimmume disease, allergies, immunodeficiencies, transplantation, graft vesus host, anemia, ataxia-telangiectasia, lymphedema, tonsilitis, hypercoagulation, and sudden infant death syndrome.

The NOV6 nucleic acid and protein of the invention, or fragments thereof, may further be useful in diagnostic applications, wherein the presence or amount of the NOV6 nucleic acid or the protein are to be assessed.

NOV7

A NOV7 sequence according to the invention includes a nucleic acid sequence encoding a polypeptide related to the sulfate anion transporter family of proteins. A NOV7 nucleic acid is likely to be expressed in the adrenal gland. A NOV7 nucleic acid and its encoded polypeptide includes the sequences shown in Table 22. The disclosed nucleic acid (SEQ ID NO:13) is 2,145 nucleotides in length and contains an open reading frame (ORF) that begins with an ATG initiation codon at nucleotides 70-72 and ends with a TAG stop codon at nucleotides 1969-1971. The representative ORF encodes a 633 amino acid polypeptide (SEQ ID NO:14) with a predicted molecular weight of 67,472.4 Da. PSORT analysis of a NOV7 polypeptide predicts a peroxisomal localization with a certainty of 0.8000. SIGNALP analysis suggests the lack of a signal peptide. Putative untranslated regions upstream and downstream of the ORF are underlined in SEQ ID NO.: 13).

TABLE 22

| | |
|---|---|
| GATCCGGGGGCTCCTGTGACCATGCCCTCTTCTCGCCCGCAGGTCGGCCACGGGACC | (SEQ ID NO.: 13) |
| TGACGCAACAGGATGGACGAGTCCCCTGAGCCTCTGCAGCAGGGCAGAGGGCCGGT | |
| GCCGGTCCGACGCCAGCGCCCAGCACCCCGGGGTCTGCGTGAGATGCTGAAGGCCA | |
| GGCTGTGGTGCAGCTGCTCGTGCAGTGTGCTGTGCGTCCGGGCGCTGGTGCAGGACC | |
| TGCTCCCCGCCACGCGCTGGCTGCGTCAGTACCGCCCGCGGGAGTACCTGGCAGGC | |
| GACGTCATGTCTGGGCTGGTCATCGGCATCATCCTGGTCCCGCAGGCCATCGCCTAC | |
| TCATTGCTGGCCGGGCTGCAGCCCATCTACAGCCTCTATACGTCCTTCTTCGCCAACC | |
| TCATCTACTTCCTCATGGGCACCTCACGGCATGTCTCCGTGGGCATCTTCAGCCTGCT | |
| TTGCCTCATGGTGGGGCAGGTGGTGGACCGGGAGCTCCAGCTGGCCGGCTTTGACCC | |
| CTCCCAGGACGGCCTGCAGCCCGGAGCCAACAGCAGCACCCTCAACGGCTCGGCTG | |
| CCATGCTGGACTGCGGGCGTGACTGCTACGCCATCCGTGTCGCCACCGCCCTCACGC | |
| TGATGACCGGGCTTTACCAGGTCCTCATGGGCGTCCTCCGGCTGGGCTTCGTGTCCG | |
| CCTACCTCTCACAGCCACTGCTCGATGGCTTTGCCATGGGGGCCTCCGTGACCATCC | |
| TGACCTCGCAGCTCAAACACCTGCTGGGCGTGCGGATCCCGCGGCACCAGGGGCCC | |
| GGCATGGTGGTCCTCACATGGCTGAGCCTGCTGCGCGGCGCCGGGCAGGCCAACGT | |
| GTGCGACGTGGTCACCAGCACGGTGTGCCTGGCGGTGCTGCTAGCCGCGAAGGAGC | |
| TCTCAGACCGCTACCGACACCGCCTGAGGGTGCCGCTGCCCACGGAGCTGCTGGTCA | |
| TCGTGGTGGCCACACTCGTGTCGCACTTCGGGCAGCTCCACAAGCGCTTTGGCTCGA | |
| GCGTGGCTGGCGACATCCCCACGGGTTTCATGCCCCCTCAGGTCCCAGAGCCCAGGC | |
| TGATGCAGCGTGTGGCTTTGGATGCCGTGGCCCTGGCCCTCGTGGCTGCCGCCTTCT | |
| CCATCTCGCTGGCGGAGATGTTCGCCCGCAGTCACGGCTACTCTGTGCGTGCCAACC | |
| AGGAGCTGCTGGCTGTGCATCGTGGTCACCTGCGGGGGGCCTGCCAAGGTGTGGGA | |
| CTCCCGGGCTGTGGCGGATCACCGGCTGACGCGCTGGTCTGGGCAGGCACGGGCAC | |
| CTGTATGCTGGTCAGCACAGAGGCCGGGCTGCTGGCTGGCGTCATCCTCTCGCTGCT | |
| CAGCCTGGCCGGCCGCACCCAAAAGCCACGCACCGCCCTGCTGGCCCGCATCGGGG | |
| ACACGGCCTTCTACGAGGATGCCACAGAGTTCGAGGGCCTCGTCCCTGAGCCCGGC | |
| GTGCGGGTGTTCCGCTTTGGGGGGCCGCTGTACTATGCCAACAAGGACTTCTTCCTG | |
| CAGTCACTCTACAGCCTCACGGGGCTGGACGCAGGGTGCATGGCTGCCAGGAGGAA | |
| GGAGGGGGGCTCAGAGACGGGGGTCGGTGAGGGAGGCCCTGCCCAGGGCGAGGAC | |
| CTGGGCCCGGTTAGCACCAGGGCTGCGCTGGTGCCCGCAGCGGCCGGCTTCCACAC | |
| AGTGGTCATCGACTGCGCCCCGCTGCTGTTCCTAGACGCAGCCGGTGTGAGCACGCT | |
| GCAGGACCTGCGCCGAGACTACGGGGCCCTGGGCATCAGCCTGCTGCTAGCCTGCT | |
| GCAGCCCGCCTGTGAGAGACATTCTGAGCAGAGGAGGCTTCCTCGGGGAGGGCCCC | |
| GGGGACACGGCTGAGGAGGAGCAGCTGTTCCTCAGTGTGCACGATGCCGTGCAGAC | |
| AGCACGAGCCCGCCACAGGGAGCTGGAGGCCACCGATGCCCATCTGTAGCAGGGCC | |
| AGGCCTGCCCAGCAGCCTCTGCTCCCTCCTGGGGACCCACAGCAGACGTCTGCAAGC | |
| CACTGCTGAGACCCTTCCCAGGGAGGAGCCACCCAAGAGCTGCACTCTTGTGCCACA | |
| GCTGCCCTGGGGAAACCGGGGAACCCCAACTGGGAAAGGAGGCCCTCTGATCA | |
| MDESPEPLQQGRGPVPVRRQRPAPRGLREMLKARLWCSCSCSVLCVRALVQDLLPATR | (SEQ ID NO.: 14) |
| WLRQYRPREYLAGDVMSGLVIGIILVPQAIAYSLLAGLQPIYSLYTSFFANLIYFLMGTSR | |
| HVSVGIFSLLCLMVGQVVDRELQLAGFDPSQDGLQPGANSSTLNGSAAMLDCGRDCYAI | |
| RVATALTLMTGLYQVLMGVLRLGFVSAYLSQPLLDGFAMGASVTILTSQLKHLLGVRIP | |
| RHQGPGMVVLTWLSLLRGAGQANVCDVVTSTVCLAVLLAAKELSDRYRHRLRVPLPTE | |
| LLVIVVATLVSHFGQLHKRFGSSVAGDIPTGFMPPQVPEPRLMQRVALDAVALALVAAA | |
| FSISLAEMFARSHGYSVRANQELLAVHRGHLRGACQGVGLPGCGGSPADALVWAGTGT | |
| CMLVSTEAGLLAGVILSLLSLAGRTQKPRTALLARIGDTAFYEDATEFEGLVPEPGVRVF | |
| RFGGPLYYANKDFFLQSLYSLTGLDAGCMAARRKEGGSETGVGEGGPAQGEDLGPVST | |
| RAALVPAAAGFHTVVIDCAPLLFLDAAGVSTLQDLRRDYGALGISLLLACCSPPVRDILSR | |
| GGFLGEGPGDTAEEEQLFSVHDAVQTARARHRELEATDAHL | |

A NOV7 nucleic acid has a high degree of homology (99% identity) with human sulfate anion transporter mRNA (SAT1; GenBank Accession No.: AF297659), as is shown in Table 23. A NOV7 polypeptide has homology (74% identity, 81% similarity) with a rat sulfate anion transporter 1 polypeptide (SAT1; SwissProt Accession No.: P45380), as is shown in Table 24.

TABLE 23

| | | | | |
|---|---|---|---|---|
| NOV7: | 40 | caggtcggccacgggacctgacgcaacaggatggacgagtcccctgagcctctgcagcag | 99 | (SEQ ID NO.: 45) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 93 | caggtcggccacgggacctgacgcaacaggatggacgagtcccctgagcctctgcagcag | 152 | (SEQ ID NO.: 46) |
| NOV7: | 100 | ggcagagggccggtgccggtccgacgccagcgcccagcaccccggggtctgcgtgagatg | 159 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 153 | ggcagagggccggtgccggtccgacggcagcgcccagcaccccggggtctgcgtgagatg | 212 | |
| NOV7: | 160 | ctgaaggccaggctgtggtgcagctgctcgtgcagtgtgctgtgcgtccgggcgctggtg | 219 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 213 | ctgaaggccaggctgtggtgcagctgctcgtgcagtgtgctgtgcgtccgggcgctggtg | 272 | |
| NOV7: | 220 | caggacctgctccccgccacgcgctggctgcgtcagtaccgcccgcgggagtacctggca | 279 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 273 | caggacctgctccccgccacgcgctggctgcgtcagtaccgcccgcgggagtacctggca | 332 | |
| NOV7: | 280 | ggcgacgtcatgtctgggctggtcatcggcatcatcctggtcccgcaggccatcgcctac | 339 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 333 | ggcgacgtcatgtctgggctggtcatcggcatcatcctggtgccgcaggccatcgcctac | 392 | |
| NOV7: | 340 | tcattgctggccgggctgcagcccatctacagcctctatacgtccttcttcgccaacctc | 399 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 393 | tcattgctggccgggctgcagcccatctacagcctctatacgtccttcttcgccaacctc | 452 | |
| NOV7: | 400 | atctacttcctcatgggcacctcacggcatgtctccgtgggcatcttcagcctgctttgc | 459 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 453 | atctacttcctcatgggcacctcacggcatgtctccgtgggcatcttcagcctgctttgc | 512 | |
| NOV7: | 460 | ctcatggtggggcaggtggtggaccgggagctccagctggccggctttgacccctcccag | 519 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 513 | ctcatggtggggcaggtggtggaccgggagctccagctggccggctttgacccctcccag | 572 | |
| NOV7: | 520 | gacggcctgcagcccggagccaacagcagcaccctcaacggctcggctgccatgctggac | 579 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 573 | gacggcctgcagcccggagccaacagcagcaccctcaacggctcggctgccatgctggac | 632 | |
| NOV7: | 580 | tgcgggcgtgactgctacgccatccgtgtcgccaccgccctcacgctgatgaccgggctt | 639 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 633 | tgcgggcgtgactgctacgccatccgtgtcgccaccgccctcacgctgatgaccgggctt | 692 | |
| NOV7: | 640 | taccaggtcctcatgaacatcctccggctgggcttcgtgtccgcctacctctcacagcca | 699 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 693 | taccaggtcctcatgggcgtcctccggctgggcttcgtgtccgcctacctctcacagcca | 752 | |
| NOV7: | 700 | ctgctcgatggctttgccatgggggcctccgtgaccatcctgacctcgcagctcaaacac | 759 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 753 | ctgctcgatggctttgccatgggggcctccgtgaccatcctgacctcgcagctcaaacac | 812 | |
| NOV7: | 760 | ctgctgggcgtgcggatcccgcggcaccaggggcccggcatggtggtcctcacatggctg | 819 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 813 | ctgctgggcgtgcggatcccgcggcaccaggggcccggcatggtggtcctcacatggctg | 872 | |
| NOV7: | 820 | agcctgctgcgcggcgccgggcaggccaacgtgtgcgacgtggtcaccagcacggtgtgc | 879 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 873 | agcctgctgcgcggcgccgggcaggccaacgtgtgcgacgtggtcaccagcacggtgtgc | 932 | |
| NOV7: | 880 | ctggcggtgctgctagccgcgaaggagctctcagaccgctaccgacaccgcctgagggtg | 939 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 933 | ctggcggtgctgctagccgcgaaggagctctcagaccgctaccgacaccgcctgagggtg | 992 | |
| NOV7: | 940 | ccgctgcccacggagctgctggtcatcgtggtggccacactcgtgtcgcacttcgggcag | 999 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 993 | ccgctgcccacggagctgctggtcatcgtggtggccacactcgtgtcgcacttcgggcag | 1052 | |
| NOV7: | 1000 | ctccacaagcgctttggctcgagcgtggctggcgacatccccacgggtttcatgccccct | 1059 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 1053 | ctccacaagcgctttggctcgagcgtggctggcgacatccccacgggtttcatgccccct | 1112 | |
| NOV7: | 1060 | caggtcccagagcccaggctgatgcagcgtgtggctttggatgccgtggccctggccctc | 1119 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 1113 | caggtcccagagcccaggctgatgcagcgtgtggctttggatgccgtggccctggccctc | 1172 | |
| NOV7: | 1120 | gtggctgccgccttctccatctcgctggcggagatgttcgcccgcagtcacggctactct | 1179 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 1173 | gtggctgccgccttctccatctcgctggcggagatgttcgcccgcagtcacggctactct | 1232 | |
| NOV7: | 1180 | gtgcgtgccaaccaggagctgctggctgtg 1209 | | |
| | | \|\|\|\|\|\|\|\|\|\|\|\|\|\|\|\|\|\|\|\|\|\|\|\|\|\|\|\|\|\| | | |
| SAT1: | 1233 | gtgcgtgccaaccaggagctgctggctgtg 1262 | | |

TABLE 24

| | | | | |
|---|---|---|---|---|
| NOV7: | 70 | MDESPEPLQQGRGPVPVRRQRPAPRGLREMLKARLWCSCSCSVLCVRALVQDLLPATRWL | 249 | (SEQ ID NO.: 47) |
| | | \|\| \|\|\|\| \|+\| \| \|\|\|\| \| +\|\| \| \|\|\|\|\| \|\|+\|\|+ \| +\|\|\|\| \| \| \|\|\| | | |
| SAT1: | 1 | MDASPEPPQKGGTLVLVRRQPPVSQGLLETLKARLKKSCTCSMPCAQALVQGLFPVIRWL | 60 | (SEQ ID NO.: 48) |
| NOV7: | 250 | RQYRPREYLAGDVMSGLVIGIILVPQAIAYSLLAGLQPIYSLYTSFFANLIYFLMGTSRH | 429 | |
| | | \|\|\| +\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SAT1: | 61 | PQYRLKEYLAGDVMSGLVIGIILVPQAIAYSLLAGLQPIYSLYTSFFANLIYFLMGTSRH | 120 | |
| NOV7: | 430 | VSVGIFSLLCLMVGQVVDRELQLAGFDPSQDGLQPGANSSTLNGSAAML----DCGRDCY | 597 | |
| | | \|+\|\|\|\|\|\|\|\|\|\|\|\|\|\|\|\|\|\|\|\|\|\|\|\|\|\|\|\|\| \| \|\| \| \|\|\|\| +\| + \|\|\|\|\|\|+ | | |
| SAT1: | 121 | VNVGIFSLLCLMVGQVVDRELQLAGFDPSQDSLGPGNNDSTLNNTATLTVGLQDCGRDCH | 180 | |
| NOV7: | 598 | AIRVATALTLMTGLYQVLMGVLRLGFVSAYLSQPLLDGFAMGASVTILTSQLKHLLGVRI | 777 | |
| | | \|\|\|+\|\|\|\|\|\|\| \|\|\|\|\|\|\|\|+\|\|\|\|\|\|\| \|\|\|\|\|\|\|\|\|\|\|\|\|\|\|\|\|\|\|\|\|\| \|\|\|\|\|\|\|\| | | |
| SAT1: | 181 | AIRIATALTLMAGLYQVLMGILRLGFVSTYLSQPLLDGFAMGASVTILTSQAKHLLGVRI | 240 | |
| NOV7: | 778 | PRHQGPGMVVLTWLSLLRGAGQANVCDVVTSTVCLAVLLAAKELSDRYRHRLRVPLPTEL | 957 | |
| | | \|\|\|\|\| \|\|\|+ \|\|\|\|\|\|+ \|\|\|\|+\|\|\|\|\|\| \|\|\|\|\|\|\| \|\|\|\|\|\|\|\|\|\| \|+\|\|+\|\|\|\| | | |
| SAT1: | 241 | PRHQGLGMVIHTWLSLLQNVGQANLCDVVTSAVCLAVLLTAKELSDRYRHYLKVPVPTEL | 300 | |
| NOV7: | 958 | LVIVVATLVSHFGQLHKRFGSSVAGDIPTGFMPPQVPEPRLMQRVALDAVALALVAAAFS | 1137 | |
| | | \|\|\|\|\|\|\|+ \|\|\|\|\|\|\| \|\|\|\|\|\|\|\|+\|\|\|\|\|+ \|\|+\|+\|+\| \|\|\|\|\|+\|\|\|\| +\|\|\| | | |
| SAT1: | 301 | LVIVVATIASHFGQLHTRFGSSVAGNIPTGFVAPQIPDPKIMWSVALDAMSLALVGSAFS | 360 | |
| NOV7: | 1138 | ISLAEMFARSHGYSVRANQELLAVHRGHLRGACQGVGLPG---CGGSPA---DALVWAGT | 1299 | |
| | | \|\|\|\|\|\|\|\|\|\|\|\|\|\|\| \|\|\|\|\|\|\|\| \| \| \|\| \| + \| \|\| \| | | |
| SAT1: | 361 | ISLAEMFARSHGYSVSANQELLAV------GCCNV--LPAFFHCFATSAALSKTLVKIAT | 412 | |
| NOV7: | 1300 | GTCMLVSTEAGLLAGVILSLLSLA 1371 | | |
| | | \| \| + \| \|+\| \|\| \|\| | | |
| SAT1: | 413 | G-CQTQLSSVVSAAVVLLVLLVLA 435 | | |

Where | indicates identity and + indicates similarity.

Where | indicates identity and + indicates similarity.

Sulfate anion transporter proteins are members of the superfamily of anion exchangers. Two vertebrate sulfate transporters that play a role in sulfate incorporation in tissues are members of the superfamily of anion exchangers: the diastrophic dysplasia sulfate transporter, which is mutant in diastrophic dysplasia and certain other skeletal dysplasias, and downregulated in adenoma, which is mutant in familial chloride diarrhea. By subtractive hybridization, Schweinfest and co-workers isolated a cDNA for a tumor suppressor candidate gene, which they called DRA (downregulated in adenoma), from a normal colon tissue cDNA library. Its expression, which appeared to be limited to the mucosa of normal colon, was significantly decreased in adenomas and adenocarcinomas of the colon and was downregulated early in tumorigenesis (See Schweinfest et al., 1993, Proc. Nat. Acad. Sci. U.S.A. 90:4166). These two sulfate transporters contain twelve membrane-spanning domains and are sensitive to the anion-exchanger inhibitor DIDS. Girard and colleagues demonstrated that HEVECs express 2 functional classes of sulfate transporters defined by their differential sensitivity to the DIDS anion-exchanger inhibitor. They reported the molecular characterization of a DIDS-resistant sulfate transporter from human HEVECs, designated SUT1 (See Girard et al., 1999, Proc. Nat. Acad. Sci. U.S.A. 96:12772). SUT1 belongs to the family of sodium-coupled anion transporters and exhibits 40 to 50% amino acid identity with the rat renal sodium/sulfate cotransporter NaSi1, as well as with the human and rat sodium/dicarboxylate cotransporters NADC1/SDCT1 and NADC3/SDCT2. Functional expression studies in cRNA-injected Xenopus laevis oocytes showed that SUT1 mediates high levels of sodium-dependent sulfate transport, which is resistant to DIDS inhibition. Northern blot analysis showed that SUT1 exhibits a highly restricted tissue distribution, with abundant expression in placenta. Reverse transcription-PCR analysis indicated that SUT1 and DTDST were coexpressed in HEVECs. SUT1 and DTDST may correspond, respectively, to the DIDS-resistant and DIDS-sensitive components of sulfate uptake in HEVEC (See Girard et al., 1999, Proc. Nat. Acad. Sci. U.S.A. 96:12772).

Girard and colleagues also mapped the SUT1 gene to 7q33 by finding a sequence tagged site (STS) corresponding to nucleotides 2579-2833 of the SUT1 cDNA. This STS mapped to chromosome 7 at D7S509, which maps to 7q33 close to 7q32. They confirmed these mapping data by identifying ESTs with sequence identity to SUT1 cDNA that mapped between markers D7S500 and D7S509 on 7q33 (See Girard et al., 1999, Proc. Nat. Acad. Sci. U.S.A. 96:12772).

NOV7 represents a new member of the sulfate anion transporter family of proteins. NOV7 is useful in determining changes in expression of genes contained within the sulfate anion transporter protein family. NOV7 satisfies a need in the art by providing new diagnostic or therapeutic compositions useful in the treatment of disorders associated with alterations in the expression of members of the sulfate anion transporter-associated protein family of proteins. NOV7 nucleic acids, polypeptides; antibodies, and other compositions of the present invention are useful in the treatment and/or diagnosis of a variety of diseases and pathologies, including by way of nonlimiting example, those involving disorders such as Pendred syndrome, skeletal dysplasias, diastrophic dysplasia, cancer, adenoma.

NOV8

A NOV8 sequence according to the invention includes a nucleic acid sequence encoding a polypeptide related to the cytostatin family of proteins. A NOV8 nucleic acid was identified on human chromosome 1. A NOV8 nucleic acid and its encoded polypeptide includes the sequences shown in Table 25. The disclosed nucleic acid (SEQ ID NO:15) is 406 nucleotides in length and contains an open reading frame (ORF) that begins with an ATG initiation codon at nucleotides 1-3 and ends with a TAA stop codon at nucleotides 397-399. The representative ORF encodes a 132 amino acid polypeptide (SEQ ID NO:16) with a predicted molecular weight of 15,599.6 Da. PSORT analysis of a NOV8 polypeptide predicts a peroxisomal localization with a certainty of 0.6400. SIGNALP analysis suggests the lack of a signal peptide. Putative untranslated regions downstream of the ORF are underlined in SEQ ID NO.: 15).

TABLE 25

(SEQ ID NO.: 15)

GTGGAGGAGGCTTTCTGTAATACCTGGAAGCTGACCGACCAGAACTTTGA
TGAGTACATGAAGGCTCTAGGGATGGGCTTTGTCACTAGGCAGGTGGGAA
ATGTGGACAAACCAAGAGTGATTATCAGTCAAGAAGAAGACAAGGTGGTG
ATCAGGATTCAAAGTATGTTCAAGAACACAGAGGTTAGTTTCCATCTGGG
AGAAGAGTTTGATGAAACCACTACAGATGACAGAAACTGCAAGTTTGTTG
TTAGTCTGGACAGAGACAAACTCATTCACATACAGAAATGGGATGACAAA
GAAACATATTTTATAAGAGAAATTAAGTATGGTGAAATGGTTATGACCTT
TACTTTTGGTGATGATGTGGTTGCCGTTCACCACTATAAGAAGGCATAAA
AATGTT

(SEQ ID NO.: 16)

VEEAFCNTWKLTDQNFDEYMKALGMGFVTRQVGNVDKPRVIISQEEDKVV
LRIQSMFKNTEVSFHLGEEFDETTTDDRNCKFVVSLDRDKLIHIQKWDDK
ETYFIREIKYGEMVMTFTFGDDVVAVLIHYKKA

A NOV8 nucleic acid has homology (88% identity) with a human cytostatin II mRNA (CYT2; Patn Accession No.: T7475 1), as is shown in Table 26. A NOV8 polypeptide has homology (80% identity, 86% similarity) with a human cytosatin II polypeptide (CYT2; PatP Accession No.: W22408), as is shown in Table 27. A NOV8 polypeptide also has homology (80% identity, 86% similarity) with a human fatty acid-binding protein (FABP; SwissProt. Accession No.: 015540), as is shown in Table 28. Expression profiling of a NOV8 nucleic acid is described in Example 6.

TABLE 26

| | | | | |
|---|---|---|---|---|
| NOV8: | 2 | TGGAGGAGGCTTTCTGTAATACCTGGAAGCTGACCGAC---CAGAACTTTGATGAGTACA | 58 | (SEQ ID NO.: 49) |
| | | \|\|\| \|\|\|\|\|\|\|\|\|\|\|\|\| \|\|\|\|\|\|\|\|\|\|\|\|\|\|\|\| \|\| \|\|\|\|\|\|\|\|\|\|\|\|\|\|\|\|\|\|\| | | |
| CYT2: | 17 | TGGTGGAGGCTTTCTGTGCTACCTGGAAGCTGACCAACAGTCAGAACTTTGATGAGTACA | 76 | (SEQ ID NO.: 50) |
| NOV8: | 59 | TGAAGGCTCTAGGGATGGGCTTTGTCACTAGGCAGGTGGGAAATGTGGACAAACCAAGAG | 118 | |
| | | \|\|\|\|\|\|\|\|\|\|\|\|\| \|\|\|\|\|\|\|\|\| \|\|\|\|\|\|\|\|\|\|\|\|\|\|\|\|\|\|\|\|\|\| \|\|\|\|\|\|\|\| \| | | |
| CYT2: | 77 | TGAAGGCTCTAGGCGTGGGCTTTGCCACTAGGCAGGTGGGAAATGTGACCAAACCAACGG | 136 | |
| NOV8: | 119 | TGATTATCAGTCAAGAAGAAGACAAGGTGGTGATCAGGATTCAAAGTATGTTCAAGAACA | 178 | |
| | | \| \|\|\|\|\|\|\|\|\|\|\|\|\| \|\|\|\|\|\|\|\|\| \|\|\|\|\| \|\|\|\|\|\|\| \|\| \|\| \| \|\|\|\|\|\|\|\|\|\| | | |
| CYT2: | 137 | TAATTATCAGTCAAGAAGGAGACAAAGTGGTCATCAGGACTCTCAGCACATTCAAGAACA | 196 | |
| NOV8: | 179 | CAGAGGTTAGTTTCCATCTGGGAGAAGAGTTTGATGAAACCACTACAGATGACAGAAACT | 238 | |
| | | \| \|\|\| \|\|\|\|\|\|\|\|\|\| \|\|\|\|\|\|\|\|\|\|\|\|\|\|\|\|\|\|\|\|\|\|\|\|\|\|\| \|\|\|\|\|\|\| \|\|\|\|\|\|\| | | |
| CYT2: | 197 | CGGAGATTAGTTTCCAGCTGGGAGAAGAGTTTGATGAAACCACTGCAGATGATAGAAACT | 256 | |
| NOV8: | 239 | GCAAGTTTGTTGTTAGTCTGGACAGAGACAAACTCATTCACATACAGAAATGGGATGACA | 298 | |
| | | \| \|\|\|\| \|\|\|\|\|\|\|\|\| \|\|\|\|\| \|\|\|\|\|\|\|\|\|\| \|\|\|\|\|\|\|\|\|\|\|\|\|\|\|\|\|\|\|\|\| \|\| | | |
| CYT2: | 257 | GTAAGTCTGTTGTTAGCCTGGATGGAGACAAACTTGTTCACATACAGAAATGGGATGGCA | 316 | |
| NOV8: | 299 | AAGAAACATATTTTATAAGAGAAATTAAGTATGGTGAAATGGTTATGACCTTTACTTTTG | 358 | |
| | | \|\|\|\|\|\|\|\| \|\|\|\|\| \|\|\|\|\|\|\|\|\|\|\|\|\|\| \|\|\|\| \|\|\|\|\|\|\|\|\|\|\|\|\|\| \|\|\|\|\|\|\|\|\| | | |
| CYT2: | 317 | AAGAAACAAATTTTGTAAGAGAAATTAAGGATGGCAAAATGGTTATGACCCTTACTTTTG | 376 | |
| NOV8: | 359 | GTGATGATGTGGTTGCCGTTCACCACTATAAGAAGGCATAAAAATGT 405 | | |
| | | \|\|\|\|\|\| \|\| \| \|\|\|\| \|\|\|\| \|\|\|\|\|\|\| \|\|\|\|\|\|\|\|\|\|\|\|\|\|\|\|\| | | |
| CYT2: | 377 | GTGATG-TG-G-TTGCTGTTCGCCACTATGAGAAGGCATAAAAATGT 420 | | |

TABLE 27

| | | | | |
|---|---|---|---|---|
| NOV8: | 7 | EAFCNTWKLTD-QNFDEYMKALGMGFVTRQVGNVDKPRVIISQEEDKVVIRIQSMFKNTE | 183 | (SEQ ID NO.: 51) |
| | | \|\|\|\| \|\|\|\|\|+ \|\|\|\|\|\|\|\|\|\|\|+\|\| \|\|\|\|\|\|\| \|\| \|\|\|\|\|\| \|\|\|\|\|\| \| \|\|\|\|\| | | |
| CYT2: | 3 | EAFCATWKLTNSQNFDEYMKALGVGFATRQVGNVTKPTVIISQEGDKVVIRTLSTFKNTE | 62 | (SEQ ID NO.: 52) |
| NOV8: | 184 | VSFHLGEEFDETTTDDRNCKFVVSLDRDKLIHIQKWDDKETYFIREIKYGEMVMTFTFGD | 363 | |
| | | +\|\| \|\|\|\|\|\|\|\|\| \|\|\|\|\|\| \|\|\|\|\| \|\|\|+\|\|\|\|\|\| \|\|\| \|+\|\|\|\| \|+\|\|\|\| \|\|\|\| | | |
| CYT2: | 63 | ISFQLGEEFDETTADDRNCKSVVSLDGDKLVHIQKWDGKETNFVREIKDGKMVMTLTFGD | 122 | |
| NOV8: | 364 | DVVAVHHYKKA 396 | | |
| | | \|\|\|\| \|\|+\|\| | | |
| CYT2: | 123 | -VVAVRHYEKA 132 | | |

Where | indicates identity and + indicates similarity.

Where | indicates identity and + indicates similarity.

TABLE 28

| | | | | |
|---|---|---|---|---|
| NOV8: | 7 | EAFCNTWKLTD-QNFDEYMKALGMGFVTRQVGNVDKPRVIISQEEDKVVIRIQSMFKNTE | 183 | (SEQ ID NO.: 53) |
| | | \|\|\|\| \|\|\|\|\|+ \|\|\|\|\|\|\|\|\|\|\|+\|\| \|\|\|\|\|\|\| \|\| \|\|\|\|\|\| \|\|\|\|\|\| \| \|\|\|\|\| | | |
| FABP: | 3 | EAFCATWKLTNSQNFDEYMKALGVGFATRQVGNVTKPTVIISQEGDKVVIRTLSTFKNTE | 62 | (SEQ ID NO.: 54) |
| NOV8: | 184 | VSFHLGEEFDETTTDDRNCKFVVSLDRDKLIHIQKWDDKETYFIREIKYGEMVMTFTFGD | 363 | |
| | | +\|\| \|\|\|\|\|\|\|\|\| \|\|\|\|\|\| \|\|\|\|\| \|\|\|+\|\|\|\|\|\| \|\|\| \|+\|\|\|\| \|+\|\|\|\| \|\|\|\| | | |
| FABP: | 63 | ISFQLGEEFDETTADDRNCKSVVSLDGDKLVHIQKWDGKETNFVREIKDGKMVMTLTFGD | 122 | |
| NOV8: | 364 | DVVAVHHYKKA 396 | | |
| | | \|\|\|\| \|\|+\|\| | | |
| FABP: | 123 | -VVAVRHYEKA 132 | | |

Where | indicates identity and + indicates similarity.

Where | indicates identity and + indicates similarity.

NOV9

A NOV9 sequence according to the invention includes a nucleic acid sequence encoding a polypeptide related to the cytostatin family of proteins. A NOV9 nucleic acid was identified on human chromosome 1. A NOV9 nucleic acid and its encoded polypeptide includes the sequences shown in Table 29. The disclosed nucleic acid (SEQ ID NO:17) is 418 nucleotides in length and contains an open reading frame (ORF) that begins with an ATG initiation codon at nucleotides 4-6 and ends with a TAA stop codon at nucleotides 409-411. The representative ORF encodes a 135 amino acid polypeptide (SEQ ID NO:18). Putative untranslated regions upstream and downstream of the ORF are underlined in SEQ ID NO.: 17.

TABLE 29

(SEQ ID NO.: 17)

ATAATGGTAAGGGTGGAGGAGGCTTTCTGTAATACCTGGAAGCTGACCGA
CCAGAACTTTGATGAGTACATGAAGGCTCTAGGGATGGGCTTTGTCACTA
GGCAGGTGGGAAATGTGGACAAACCAAGAGTGATTATCAGTCAAGAAGAA
GACAAGGTGGTGATCAGGATTCAAAGTATGTTCAAGAACACAGAGGTTAG
TTTCCATCTGGGAGAAGAGTTTGATGAAACCACTACAGATGACAGAAACT
GCAAGTTTGTTGTTAGTCTGGACAGAGACAAACTCATTCACATACAGAAA
TGGGATGACAAAGAAACATATTTTATAAGAGAAATTAAGTATGGTGAAAT
GGTTATGACCTTTACTTTTGGTGATGATGTGGTTGCCGTTCACCACTATA
AGAAGGCATAAAAATGTT

(SEQ ID NO.: 18)

MVRVEEAFCNTWKLTDQNFDEYMKALGMGFVTRQVGNVDKPRVIISQEED
KVVIRIQSMFKNTEVSFHLGEEFDETTTDDRNCKFVVSLDRDKLIHIQKW
DDKETYFIREIKYGEMVMTFTFGDDVVAVHHYKKA

A NOV9 nucleic acid has homology (88% identity) with a human cytostatin II mRNA (CYT2; Patn Accession No.: T74751). A NOV9 polypeptide has homology (80% identity, 86% similarity) with a human cytosatin II polypeptide (CYT2; PatP Accession No.: W22408). A NOV9 polypeptide also has homology (80% identity, 86% similarity) with a human fatty acid-binding protein (FABP; SwissProt. Accession No.: 015540). A region of a NOV9 polypeptide also has a high degree of homology (100%) with NOV8, as is shown in Table 30.

TABLE 30

| | | | | |
|---|---|---|---|---|
| NOV9: | 4 | VEEAFCNTWKLTDQNFDEYMKALGMGFVTRQVGNVDKPRVIISQEEDKVVIRIQSMFKNT | 63 | (SEQ ID NO.: 55) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| NOV8: | 1 | VEEAFCNTWKLTDQNFDEYMKALGMGFVTRQVGNVDKPRVIISQEEDKVVIRIQSMFKNT | 60 | (SEQ ID NO.: 56) |
| NOV9: | 64 | EVSFHLGEEFDETTTDDRNCKFVVSLDRDKLIHIQKWDDKETYFIREIKYGEMVMTFTFG | 123 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| NOV8: | 61 | EVSFHLGEEFDETTTDDRNCKFVVSLDRDKLIHIQKWDDKETYFIREIKYGEMVMTFTFG | 120 | |
| NOV9: | 124 | DDVVAVHHYKKA 135 | | |
| | | \|\|\|\|\|\|\|\|\|\|\|\| | | |
| NOV8: | 121 | DDVVAVHHYKKA 132 | | |

Where | indicates identity.

Where | indicates identity.

Cytostatin, which was originally isolated from a microbial cultured broth as a low molecular weight inhibitor of cell adhesion to extracellular matrix (ECM), has anti-metastatic activity against B16 melanoma cells in vivo. Inhibition of cell adhesion to ECM by cytostatin has been evaluated (See Kawada et al., 1999, Biochim. Biophys. Acta 1452:209). Cytostatin inhibited tyrosine phosphorylation of focal adhesion kinase (FAK) and paxillin upon B16 cell adhesion to fibronectin. While the amount of FAK was not affected by cytostatin, electrophoretically slow-migrating paxillin appeared. Alkaline phosphatase treatment diminished cytostatin-induced slow-migrating paxillin. Furthermore, cytostatin increased intracellular serine/threonine-phosphorylated proteins and was found to be a selective inhibitor of protein phosphatase 2A (PP2A). Cytostatin inhibited PP2A with an IC(50) of 0.09 microgram/ml in a non-competitive manner against a substrate, p-nitrophenyl phosphate, but it had no apparent effect on other protein phosphatases including PP1, PP2B and alkaline phosphatase even at 100 microgram/ml. On the contrary, dephosphocytostatin, a cytostatin analogue, without inhibitory effect on PP2A did not affect B16 cell adhesion including FAK and paxillin. These results indicate that cytostatin inhibits cell adhesion through modification of focal contact proteins such as paxillin by inhibiting a PP2A type protein serine/threonine phosphatase.

Differential induction of apoptosis by cytostatin vis-à-vis another apoptosis inducer-bactobolin has been analyzed. Since, most solid tumor cells are less sensitive to apoptosis induced by anticancer drugs than hematopoietic cancer cells, Kawada and co-workers used B16 melanoma and EL-4 lymphoma cells as models for solid tumor- and hematopoietic cancer-derived cell lines respectively. It was found that apoptosis in B16 cells was induced strongly by bactobolin, but weakly by cytostatin. In contrast, apoptosis in EL-4 cells was induced strongly by cytostatin, but weakly by bactobolin. (See Kawada et al., 1999, Jpn. J. Cancer Res. 90:219).

The nucleotide sequence encoding Human cytostatin can be used for inhibiting cell growth and modulate cellular differentiation. The cytostatin II polypeptides encoded by the gene can be used for inhibiting tumour growth in a subject, for stimulating growth of or protecting nervous system cells from toxic agents or for protecting against or treating viral or microbial infections in mammals. The activity of haematopoiesis by cytostatins indicate a possible immunosuppressive activity or a lineage specific stimulation of haematopoiesis. Cytostatins thus could be used for treating conditions requiring immunosuppression. Antagonists to cytostatin may be used in vitro or in vivo to induce deficiencies or enhancement in the immune or in the haematopoietic systems. They may be used e.g. to treat cardiac myocyte hypertrophy or leukemia. The cytostatin gene product can also be used to modulate angiogenesis, to inhibit metastasis of various cancers including but not limited to breast cancer, brain and other tumors. The cytostatin polypeptide can be used amongst other things to modulate breast development and milk production. The retinoid binding potential of cytostatin derived polypetides may be used on photo receptor cells in vivo or in vitro. The cytostatin polypeptides might also be used in cerebella granular cells and photo receptor cells to provide protection from lipid peroxidation associated with the oxidative stress induced during early stages of ischemia, apoptosis, and excitatory amino acid induced cell death.

NOV8-9 represent two new members of the cytostatin family of proteins. The high degree of homology between NOV8 and NOV9 indicates that NOV8-9 consitute a new sub-family of the cytostatin family of proteins, and are useful to identify sub-family-specific binding proteins. NOV8-9 are useful in determining changes in expression of genes contained within the cytostatin protein family. NOV8-9 satisfies a need in the art by providing new diagnostic or therapeutic compositions useful in the treatment of disorders associated with alterations in the expression of members of the cytostatin-associated protein family of proteins. NOV8-9 nucleic acids, polypeptides, antibodies, and other compositions of the present invention are useful in the treatment and/or diagnosis of a variety of diseases and pathologies, including by way of nonlimiting example, those involving disorders characterized by altered cell shape, motility, and apoptosis, e.g. cancer and ischemic injury.

NOV10

A NOV10 sequence according to the invention includes a nucleic acid sequence encoding a polypeptide related to the chemokine receptor family of proteins. A NOV10 nucleic acid was identified on human chromosome 1. A NOV10 nucleic acid and its encoded polypeptide includes the sequences shown in Table 31. The disclosed nucleic acid (SEQ ID NO:19) is 1,119 nucleotides in length and contains an open reading frame (ORF) that begins with an ATG initiation codon at nucleotides 1-3 and ends with a TGA stop codon at nucleotides 1,117-1,119. The representative ORF encodes a 372 amino acid polypeptide (SEQ ID NO:20) with a predicted molecular weight of 42,793.9 Da. PSORT analysis of a NOV10 polypeptide predicts a plasma membrane protein with a certainty of 0.6400. SIGNALP analysis suggests the presence of a signal peptide with the most likely cleavage site occuring between positions 47 and 48 of SEQ ID NO.: 20).

TABLE 31

| | |
|---|---|
| ATGGAGCACACGCACGCCCACCTCGCAGCCAACAGCTCGCTGTCTTGGTGGTCCCCCG | (SEQ ID NO.: 19) |
| GCTCGGCCTGCGGCTTGGGTTTCGTGCCCGTGGTCTACTACAGCCTCTTGCTGTGCCT | |
| CGGTTTACCAGCAAATATCTTGACAGTGATCATCCTCTCCCAGCTGGTGGCAAGAAGA | |
| CAGAAGTCCTCCTACAACTATCTCTTGGCACTCGCTGCTGCCGACATCTTGGTCCTCT | |
| TTTTCATAGTGTTTGTGGACTTCCTGTTGGAAGATTTCATCTTGAACATGCAGATGCC | |
| TCAGGTCCCCGACAAGATCATAGAAGTGCTGGAATTCTCATCCATCCACACCTCCATA | |
| TGGATTACTGTACCGTTAACCATTGACAGGTATATCGCTGTCTGCCACCCGCTCAAGT | |
| ACCACACGGTCTCATACCCAGCCCGCACCCGGAAAGTCATTGTAAGTGTTTACATCAC | |
| CTGCTTCCTGACCAGCATCCCCTATTACTGGTGGCCCAACATCTGGACTGAAGACTAC | |
| ATCAGCACCTCTGTGCATCACGTCCTCATCTGGATCCACTGCTTCACCGTCTACCTGG | |
| TGCCCTGCTCCATCTTCTTCATCTTGAACTCAATCATTGTGTACAAGCTCAGGAGGAA | |
| GAGCAATTTTCGTCTCCGTGGCTACTCCACGGGGAAGACCACCGCCATCTTGGTTCAC | |
| CATTACCTCCATCTTTGCCACACTTTGGGCCCCCCCGCATCTCATGATTCTTTACCAC | |
| CTCTATGGGGCGCCCATCCAGAACCGCTGGCTGGTACACATCATGTCCGACATTGCCA | |
| ACATGCTAGCCCTTCTGAACACAGCCATCAACTTCTTCCTCTACTGCTTCATCAGCAA | |
| GCGGTTCCGCACCATGGCAGCCGCCACGCTCAAGGCTTTCTTCAAGTGCCAGAAGCAA | |
| CCTGTACAGTTCTACACCAATCATAACTTTTCCATAACAAGTAGCCCCTGGATCTCGC | |
| CGGCAAACTCACACTGCATCAAGATGCTGGTGTACCAGTATGACAAAAATGGAAAACC | |
| TATAAAAAGTCGTAATGACAGCAAAAGCTCCTACCAGTTTGAAGATGCCATTGGAGCT | |
| TGTGTCATCATCCTGTGA | |
| MEHTHAHLAANSSLSWWSPGSACGLGFVPVVYYSLLLCLGLPANILTVIILSQLVARR | (SEQ ID NO.: 20) |
| QKSSYNYLLALAAADILVLFFIVFVDFLLEDFILNMQMPQVPDKIIEVLEFSSIHTSI | |
| WITVPLTIDRYIAVCHPLKYHTVSYPARTRKVIVSVYITCFLTSIPYYWWPNIWTEDY | |
| ISTSVHHVLIWIHCFTVYLVPCSIFFILNSIIVYKLRRKSNFRLRGYSTGKTTAILFT | |
| ITSIFATLWAPRIIMILYHLYGAPIQNRWLVHIMSDIANMLALLNTAINFFLYCFISK | |
| RFRTMAAATLKAFFKCQKQPVQFYTNHNFSITSSPWISPANSHCIKMLVYQYDKNGKP | |
| IKSRNDSKSSYQFEDAIGACVIIL | |

A NOV10 polypeptide has homology (29% identity, 51% similarity) with a human chemokine receptor type I (HCR1; SwissProt Accession No.: P32246), as is shown in Table 32.

TABLE 32

| | | | | |
|---|---|---|---|---|
| NOV10: | 22 | ACGLGFVPVVYYSLLLCLGLPANILTVIILSQLVARRQKSSYNYLLALAAADILVLFFIV | 81 | (SEQ ID NO.: 57) |
| | | \| \| +\| +\| \|\|+ +\|\| \|\|\| \|++\| \| + +\| \|\|\| \|\| +\|+\| \|\| + | | |
| HCR1: | 22 | AFGAQLLPPLY-SLVFVIGLVGNILVVLVLVQYKRLKNMTSI-YLLNLAISDLLFLFTLP | 88 | (SEQ ID NO.: 58) |
| NOV10: | 82 | F-VDFLL-EDFILNMQMPQVPDKIIEVLEFSSIHTSIWITVPLTIDRYIAVCHP---LKY | 137 | |
| | | \| +\|+ \| +\|++ \| \|\|+ ++ +++ \|+ + \|\|\|\|\|\|+\|+ \| \|+ | | |
| HCR1: | 89 | FWIDYKLKDDWVFGDAMC----KILSGFYYTGLYSEIFFIILLTIDRYLAIVHAVFALRA | 144 | |
| NOV10: | 138 | HTVSYPARTRKVIVSVYITCFLTSIP-YYWWPNIWTEDYISTSVH--HVLI--W------ | 186 | |
| | | \|\|++ \| +\| ++ \| \| \|+\| \|+ \| + + \|+\| \| + \| | | |
| HCR1: | 145 | RTVTFGVITSIIIWALAI---LASMPGLYFSKTQWEFTHHTCSLHFPHESLREWKLFQAL | 201 | |
| NOV10: | 187 | -IHCFTVYLVPCSIFFILNSIIVYKLRRKSNFRLRGYSTGKTTAILFTITSIFATLWAPR | 244 | |
| | | ++ \| + \| \| + \| + \|+ \| \|+ \| + \| ++\| \| \|\| \| \| | | |
| HCR1: | 202 | KLNLFGLVL-PLLVMIICYTGIIKILLRRPNEK-----KSKAVRLIFVIMIIFFLFWTPY | 255 | |
| NOV10: | 245 | IIMILYHLYGAPI------QNRWLVHIMSDIANMLALLNTAINFFLYCFISKRFR 294 | | |
| | | + \|\| ++ + \|+\| \| + + ++\| + +\| +\| \|+ +\|\|\| | | |
| HCR1: | 256 | NLTILISVFQDFLFTHECEQSRHL-DLAVQVTEVIAYTHCCVNPVIYAFVGERFR 309 | | |

Where | indicates identity and + indicates similarity.

Where | indicates identity and + indicates similarity.

NOV11

A NOV11 sequence according to the invention includes a nucleic acid sequence encoding a polypeptide related to the chemokine receptor family of proteins. A NOV11 nucleic acid was identified on human chromosome 1. A NOV11 nucleic acid and its encoded polypeptide includes the sequences shown in Table 33. The disclosed nucleic acid (SEQ ID NO:21) is 1,343 nucleotides in length and contains an open reading frame (ORF) that begins with an ATG initiation codon at nucleotides 2-4 and ends with a TGA stop codon at nucleotides 1.061-1,063. The representative ORF encodes a 353 amino acid polypeptide (SEQ ID NO:22). PSORT analysis of a NOV11 polypeptide predicts a plasma membrane protein with a certainty of 0.6400. SIGNALP analysis suggests the presence of a signal peptide with the most likely cleavage site occuring between positions 47 and 48 of SEQ ID NO.: 22. Putative untranslated regions upstream and downstream of the ORF are underlined in SEQ ID NO.: 21.

TABLE 33

| | |
|---|---|
| TATGGAGCACACGCACGCCCACCTCGCAGCCAACAGCTCGCTGTCTTGGTGGTCCCCCGG | (SEQ ID NO.: 21) |
| CTCGGCCTGCGGCTTGGGTTTCGTGCCCGTGGTCTACTACAGCCTCTTGCTGTGCCTCGG | |
| TTTACCAGCAAATATCTTGACAGTGATCATCCTCTCCCAGCTGGTGGCAAGAAGACAGAA | |
| GTCCTCCTACAACTATCTCTTGGCACTCGCTGCTGCCGACATCTTGGTCCTCTTTTTCAT | |
| AGTGTTTGTGGACTTCCTGTTGGAAGATTTCATCTTGAACATGCAGATGCCTCAGGTCCC | |
| CGACAAGATCATAGAAGTGCTGGAATTCTCATCCATCCACACCTCCATATGGATTACTGT | |
| ACCGTTAACCATTGACAGGTATATCACTGTCTGCCACCCGCTCAAGTACCACACGGTCTC | |
| ATACCCAGCCCGCACCCGGAAAGTCATTGTAAGTGTTTACATCACCTGCTTCCTGACCAG | |
| CATCCCCTATTACTGGTGGCCCAACATCTGGACTGAAGACTACATCAGCACCTCTGTGCA | |
| TCACGTCCTCATCTGGATCCACTGCTTCACCGTCTACCTGGTGCCCTGCTCCATCTTCTT | |
| CATCTTGAACTCAATCATTGTGTACAAGCTCAGGAGGAAGAGCAATTTTCGTCTCCGTGG | |
| CTACTCCACGGGGAAGACCACCGCCATCTTGTTCACCATTACCTCCATCTTTGCCACACT | |
| TTGGGCCCCCCGCATCATCATGATTCTTTACCACCTCTATGGGGCGCCCATCCAGAACCG | |
| CTGGCTGGTACACATCATGTCCGACATTGCCAACATGCTAGCCCTTCTGAACACAGCCAT | |
| CAACTTCTTCCTCTACTGCTTCATCAGCAAGCGGTTCCGCACCATGGCAGCCGCCACGCT | |
| CAAGGCTTTCTTCAAGTGCCAGAAGCAACCTGTACAGTTCTACACCAATCATAACTTTTC | |
| CATAACAAGTAGCCCCTGGATCTCGCCGGCAAACTCACACTGCATCAAGATGCTGGTGTA | |
| CCAGTATGACAAAAATGGAAAACCTATAAAAGTATCCCCGTGATTCCATAGGTGTGGCAA | |
| CTACTGCCTCTGTCTAATCCATTTCCAGATGGGAAGGTGTCCCATCCTATGGCTGAGCAG | |
| CTCTCCTTAAGAGTGCTAATCCGATTTCCTGTCTCCCGCAGACTGGGCAATTCTCAGACT | |
| GGTAGATGAGAAGAGATGGAAGAGAAGAAAGGAGAGCATGAAGCTTGTTTTTACTTATGC | |
| ATTTATTTCCACAGAGTCGTAATGACAGCAAAAGCTCCTACCAGTTTGAAGATGCCATTG | |
| GAGCTTGTGTCATCATCCTGTGA | |
| MEHTHAHLAANSSLSWWSPGSACGLGFVPVVYYSLLLCLGLPANILTVIILSQLVARRQK | (SEQ ID NO.: 22) |
| SSYNYLLALAAADILVLFFIVFVDFLLEDFILNMQMPQVPDKIIEVLEFSSIHTSIWITV | |
| PLTIDRYITVCHPLKYHTVSYPARTRKVIVSVYITCFLTSIPYYWWPNIWTEDYISTSVH | |
| HVLIWIHCFTVYLVPCSIFFILNSIIVYKLRRKSNFRLRGYSTGKTTAILFTTTSIFATL | |
| WAPRIIMILYHLYGAPIQNRWLVHIMSDIANMLALLNTAINFFLYCFISKRFRTMAAATL | |
| KAFFKCQKQPVQFYTNHNFSITSSPWISPANSHCIKMLVYQYDKNGKPIKVSP | |

A NOV11 polypeptide has homology (29% identity, 51% similarity) with a human chemokine receptor type I (HCR1; SwissProt Accession No.: P32246). NOV11 also has a high degree of homology (99% identity) with a NOV10 polypeptide, as is shown in Table 34. Expression profiling of a NOV11 nucleic acid is described in Example 5.

TABLE 34

| | | | | |
|---|---|---|---|---|
| NOV11: | 1 | MEHTHAHLAANSSLSWWSPGSACGLGFVPVVYYSLLLCLGLPANILTVIILSQLVARRQK | 60 | (SEQ ID NO.: 59) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| NOV10: | 1 | MEHTHAHLAANSSLSWWSPGSACGLGFVPVVYYSLLLCLGLPANILTVIILSQLVARRQK | 60 | (SEQ ID NO.: 60) |
| NOV11: | 61 | SSYNYLLALAAADILVLFFIVFVDFLLEDFILNMQMPQVPDKIIEVLEFSSIHTSIWITV | 120 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| NOV10: | 61 | SSYNYLLALAAADILVLFFIVFVDFLLEDFILNMQMPQVPDKIIEVLEFSSIHTSIWITV | 120 | |
| NOV11: | 121 | PLTIDRYITVCHPLKYHTVSYPARTRKVIVSVYITCFLTSIPYYWWPNIWTEDYISTSVH | 180 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| NOV10: | 121 | PLTIDRYITVCHPLKYHTVSYPARTRKVIVSVYITCFLTSIPYYWWPNIWTEDYISTSVH | 180 | |
| NOV11: | 181 | HVLIWIHCFTVYLVPCSIFFILNSIIVYKLRRKSNFRLRGYSTGKTTAILFTITSIFATL | 240 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| NOV10: | 181 | HVLIWIHCFTVYLVPCSIFFILNSIIVYKLRRKSNFRLRGYSTGKTTAILFTITSIFATL | 240 | |
| NOV11: | 241 | WAPRIIMILYHLYGAPIQNRWLVHIMSDIANMLALLNTAINFFLYCFISKRFRTMAAATL | 300 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| NOV10: | 241 | WAPRIIMILYHLYGAPIQNRWLVHIMSDIANMLALLNTAINFFLYCFISKRFRTMAAATL | 300 | |
| NOV11: | 301 | KAFFKCQKQPVQFYTNHNFSITSSPWISPANSHCIKMLVYQYDKNGKPIK 350 | | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| NOV10: | 301 | KAFFKCQKQPVQFYTNHNFSITSSPWISPANSHCIKMLVYQYDKNGKPIK 350 | | |

Where | indicates identity.

Where | indicates identity.

Chemokine receptors are G protein-coupled receptors that mediate migration and activation of leukocytes as an important part of a protective immune response to injury and infection (See Rojo et al., 1999 Biol. Res. 32:263). In addition, chemokine receptors are used by HIV-1 to infect CD4 positive cells. The structural bases of chemokine receptor recognition and signal transduction are currently being investigated. High-resolution X-ray diffraction and NMR spectroscopy of chemokines indicate that all these peptides exhibit a common folding pattern, in spite of its low degree of primary-sequence homology. Chemokines' functional motifs have been identified by mutagenesis studies, and a possible mechanism for receptor recognition and activation is proposed, but high-resolution structure data of chemokine receptors is not yet available. Studies with receptor chimeras have identified the putative extracellular domains as the major selectivity determinants. Single-amino acid substitutions in the extracellular domains produce profound changes in receptor specificity, suggesting that motifs in these domains operate as a restrictive barrier to a common activation motif. Similarly HIV-1 usage of chemokine receptors involve interaction of one or more extracellular domains of the receptor with conserved and variable domains on the viral envelope protein gp 120, indicating a highly complex interaction. Elucidating the structural requirements for receptor interaction with chemokines and with HIV-1 will provide important insights into understanding the mechanisms of chemokine recognition and receptor activation. In addition, this information can greatly facilitate the design of effective immunomodulatory and anti-HIV-1 therapeutic agents.

Chemokines are a superfamily of small cytokine-like molecules which have been described primarily on the basis of their ability to mediate the migration of various cell types, particularly those of lymphoid origin (See Zlotnick A, et. al.; 1999, Crit Rev Immunol. 19:1). The receptors for these molecules are all seven-transmembrane domain G protein-coupled receptors that have historically been excellent targets for small-molecule drugs. This fact, coupled with the advent of large-scale DNA database mining and the recognition that chemokine receptors are also coreceptors for HIV, has driven discovery in this field at a tremendous rate. This process has included not just an expansion of the number of known chemokines and chemokine receptors, but also a greater appreciation for the variety of functions that chemokines are involved in.

Chemokines and chemokine receptors have emerged as crucial factors controlling the development and function of leukocytes (See Pelchen-Matthews A, et. al.; 1999, Immunol Rev. 168:33). Recent studies have indicated that, in addition to these essential roles, both chemokines and chemokine receptors play critical roles in viral infection and replication. Not only are chemokine receptors key components of the receptor/fusion complexes of primate immunodeficiency viruses, but chemokines can also influence virus entry and infection. Many viruses, in particular herpesviruses, encode chemokines and chemokine receptors that influence the replication of both the parent virus and other unrelated viruses. The cell surface expression of the chemokine receptors is regulated through their interaction with membrane trafficking pathways. Ligands induce receptor internalization and downmodulation through endocytosis, and recycling is regulated within endosomes. Part of the mechanism through which chemokines protect cells from HIV infection is through ligand-induced internalization of the specific chemokine receptor co-receptors. In addition, mechanisms may exist to regulate the trafficking of newly synthesized receptors to the cell surface.

Eosinophils play a central role in the pathophysiology of allergic disease (See Simon L, et al., 2000, Immunol Cell Biol 78:415). The mechanisms that regulate eosinophil migration are complex; however, chemokines and cytokines produced in both the early and late phases of the asthmatic response appear to cooperate in eosinophil recruitment. In particular, there exists a unique synergy between eotaxin and IL-5. The role of chemokine/cytokine cooperativity has been investigated in the extracellular matrix, adhesion molecule/integrin interactions, receptor polarization and aggregation and the convergence and divergence of intracellular signalling pathways. Understanding the mechanisms whereby eosinophils migrate will allow the development of specific therapeutic strategies aimed at attenuating specific components of the allergic response.

New information about the role of tissue inflammation in the pathogenesis of multiple sclerosis (MS) has driven a search for effective and specific therapeutics that address leukocyte trafficking (See Ransohoff R M, et. al.; 2000, Expert Opin Investig Drugs 9:1079). These developments in understanding MS are complemented by advances in clarifying the molecular mechanisms of leukocyte extravasation and providing the knowledge base needed to modulate tissue inflammation. Of particular interest are the chemokines and their receptors. Chemokines constitute a large family of chemoattractant peptides that regulate the vast spectrum of leukocyte migration events

NOV10 and NOV11 represent a new subfamily of the chemokine family of proteins. NOV10-11 are useful in determining changes in expression of genes contained within the chemokine protein family. NOV10-11 satisfy a need in the art by providing new diagnostic or therapeutic compositions useful in the treatment of disorders associated with alterations in the expression of members of the chemokine-associated protein family of proteins. NOV10-11 nucleic acids, polypeptides, antibodies, and other compositions of the present invention are useful in the treatment and/or diagnosis of a variety of diseases and pathologies, including by way of nonlimiting example, those involving disorders characterized by altered response to pathogens, e.g. HIV and hepatitis, and neuroepithelial disorders, e.g. dysplasia, carcinoma, and injury resulting from trauma and surgury.

NOV12

A NOV12 sequence according to the invention includes a nucleic acid sequence encoding a polypeptide related to the carboxypeptidase family of proteins. A NOV12 nucleic acid and its encoded polypeptide includes the sequences shown in Table 35. The disclosed nucleic acid (SEQ ID NO:23) is 2,392 nucleotides in length and contains an open reading frame (ORF) that begins with an ATG initiation codon at nucleotides 233-235 and ends with a TGA stop codon at nucleotides 2,283-2,185. The representative ORF encodes a 650 amino acid polypeptide (SEQ ID NO:24) with a predicted molecular weight of 74,326.3 Da. PSORT analysis of a NOV12 polypeptide predicts a mitochondrial matrix localization with a certainty of 0.4513. SIGNALP analysis suggests the lack of a signal peptide.

TABLE 35

| | |
|---|---|
| TCGGCGCGAGGATTCAGTGGATGAAGAGTACTTATTGCTAGAATGTTCTTCCTCATATGAACTTGACAACGTTCTGCTCTCTAATT | (SEQ ID NO.: 23) |
| CCATTTATTTAGCTGTTTCGAATTGATGAGGATGCAGCGAGGAGCTGCCATCTGTGAAATGGGCCCTCACCAGACTCCGAATCTGC | |
| CAGTATCTTGCTCTTGGGACTTCCAGCCTCCGGAACTGTAAACACAGCAACAAAAAAGTTATGAGAACCAAGAGCTCTGAGAAGGC | |
| TGCCAACGATGATCACAGTGTCCGTGTGGCCCGTGAAGATGTCAGAGAGAGTTGCCCACCTCTTGGTCTGGAAACCTTAAAAATCA | |
| CAGACTTCCAGCTCCATGCCTCCACGGTGAAGCGCTATGGCCTGGGGGCACATCGAGGGAGACTCAACATCCAGGCGGGCATTAAT | |
| GAAAATGATTTTTATGACGGAGCGTGGTGCGCGGGAAGAAATGACCTCCAGCAGTGGATTGAAGTGGATGCTCGGCGCCTGACCAG | |
| ATTCACTGGTGTCATCACTCAAGGGAGGAACTCCCTCTGGCTGAGTGACTGGGTGACATCCTATAAGGTCATGGTGAGCAATGACA | |
| GCCACACGTGGGTCACTGTTAAGAATGGATCTGGAGACATGATATTTGAGGGAAACAGTGAGAAGGAGATCCCTGTTCTCAATGAG | |
| CTACCCGTCCCCATGGTGGCCCGCTACATCCGCATAAACCCTCAGTCCTGGTTTGATAATGGGAGCATCTGCATGAGAATGGAGAT | |
| CCTGGGCTGCCCACTGCCAGATCCTAATAATTATTATCACCGCCGGAACGAGATGACCACCACTGATGACCTGGATTTTAAGCACC | |
| ACAATTATAAGGAAATGCGCCAGGTACAGTTGATGAAAGTTGTGAATGAAATGTGTCCCAATATCACCAGAATTTACAACATTGGA | |
| AAAAGCCACCAGGGCCTGAAGCTGTATGCTGTGGAGATCTCAGATCACCCTGGGGAGCATGAAGTCGGTGAGCCCGAGTTCCACTA | |
| CATCGCGGGGGCCCACGGCAATGAGGTGCTGGGCCGGGAGCTGCTGCTGCTGCTGGTGCAGTTCGTGTGTCAGGAGTACTTGGCCC | |
| GGAATGCGCGCATCGTCCACCTGGTGGAGGAGACGCGGATTCACGTCCTCCCCTCCCTCAACCCCGATGGCTACGAGAAGGCCTAC | |
| GAAGGGGGCTCGGAGCTGGGAGGCTGGTCCCTGGGACGCTGGACCCACGATGGAATTGACATCAACAACAACTTTCCTGATTTAAA | |
| CACGCTGCTCTGGGAGGCAGAGGATCGACAGAATGTCCCCAGGAAAGTTCCCAATCACTATATTGCAATCCCTGAGTGGTTTCTGT | |
| CGGAAAATGCCACGGTGGTGGCTGCCGAGACCAGAGCAGTCATAGCCTGGATGGAAAAAATCCCTTTTGTGCTGGGCGGCAACCTG | |
| CAGGGCGGCGAGCTGGTGGTGGCGTACCCCTACGACCTGGTGCGGTCCCCCTGGAAGACGCAGGAACACACCCCCACCCCCGACGA | |
| CCACGTGTTCCGCTGGCTGGCCTACTCCTATGCCTCCACACACCGCCTCATGACAGACGCCCGGAGGAGGGTGTGCCACACGGAGG | |
| ACTTCCAAAAGGAGGAGGGCACTGTCAATGGGGCCTCCTGGCACACCGTCGCTGGAAGTCTGAACGATTTCAGCTACCTTCATACA | |
| AACTGCTTCGAACTGTCCATCTACGTGGGCTGTGATAAATACCCACATGAGAGCCAGCTGCCCGAGGAGTGGGAGAATAACCGGGA | |
| ATCTCTGATCGTGTTCATGGAGCAGGTTCATCGTGGCATTAAAGGCTTGGTGAGAGATTCACATGGAAAAGGAATCCCAAACGCCA | |
| TTATCTCCGTAGAAGGCATTAACCATGACATCCGAACAGCCAACGATGGGGATTATCTGGCGCCTCCTGAACCCTGGAGAGTATGT | |
| GGTCACAGCAAAGCCGAAGGTTTCACTGCATCCACCAAGAACTGTATGGTTGGCTATGACATGGGGCCACAAGGTGTGACTTCACA | |
| CTTAGCAAAAACCAACATGGCCAGGATCCGAGAGATCATGGAGAAGTTTGGGAAGCAGCCCGTCAGCCTGCCAGCCAGGCGGCTGA | |
| AGCTGCGGGGGCGGAAGAGACGACAGCGTGGGTGACCCTCCTGGGCCCTTGAGACTCGTCTGGGACCCATGCAAATTAAACCAACC | |
| TGGTAGTAGCTCCATAGTGGACTCACTCACTGTTGTTTCCTCTGTAATTCAAGAAGTGCCTGGAAGAGAGGGTGCATTGTGAGGCA | |
| GGTCCCAAAAGGGAAGGCTGGAGGCTGAGGCTGTTTTCTTTTCTTTGTTCCCATTTATCCAAATAACTTG | |
| MRTKSSEKAANDDHSVRVAREDVRESCPPLGLETLKITDFQLHASTVKRYGLGAHRGRLNIQAGINENDFYDGAWCAGRNDLQQWI | (SEQ ID NO.: 24) |
| EVDARRLTRFTGVITQGRNSLWLSDWVTSYKVMVSNDSHTWVTVKNGSGDMIFEGNSEKEIPVLNELPVPMVARYIRINPQSWFDN | |
| GSICMRMEILGCPLPDPNNYYHRRNEMTTTDDLDFKHHNYKEMRQVQLMKVVNEMCPNITRIYNIGKSHQGLKLYAVEISDHPGEH | |
| EVGEPEFHYIAGAHGNEVLGRELLLLLVQFVCQEYLARNARIVHLVEETRIHVLPSLNPDGYEKAYEGGSELGGWSLGRWTHDGID | |
| INNNFPDLNTLLWEAEDRQNVPRKVPNHYIAIPEWFLSENATVVAAETRAVIAWMEKIPFVLGGNLQGGELVVAYPYDLVRSPWKT | |
| QEHTPTPDDHVFRWLAYSYASTHRLMTDARRRVCHTEDFQKEEGTVNGASWHTVAGSLNDFSYLHTNCFELSIYVGCDKYPHESQL | |
| PEEWENNRESLIVFMEQVHRGIKGLVRDSHGKGIPNAIISVEGINHDIRTANDGDYWRLLNPGEYVVTAKAEGFTASTKNCMVGYD | |
| MGATRCDFTLSKTNMARIREIMEKFGKQPVSLPARRLKLRGRKRRQRG | |

A NOV12 polypeptide has a high degree of homology (99% identity, 99% similarity) with a human membrane-bound protein PRO1310 polypeptide (P1310; PatP Accession No.: Y66645), as is shown in Table 36. Also, a NOV12 polypeptide has a high degree of homology (94% identity, 97% similarity) with a human lung tumor-specific antigen polypeptide (HLTA; PatP Accession No.: B44409), as is shown in Table 37.

TABLE 36

| | | | | |
|---|---|---|---|---|
| NOV12: | 212 | KHSNKKVMRTKSSEKAANDDHSVRVAREDVRESCPPLGLETLKITDFQLHASTVKRYGLG | 391 | (SEQ ID NO.: 61) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| P1310: | 103 | KHSNKKVMRTKSSEKAANDDHSVRVAREDVRESCPPLGLETLKITDFQLHASTVKRYGLG | 162 | (SEQ ID NO.: 62) |
| NOV12: | 392 | AHRGRLNIQAGINENDFYDGAWCAGRNDLQQWIEVDARRLTRFTGVITQGRNSLWLSDWV | 571 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| P1310: | 163 | AHRGRLNIQAGINENDFYDGAWCAGRNDLQQWIEVDARRLTRFTGVITQGRNSLWLSDWV | 222 | |
| NOV12: | 572 | TSYKVMVSNDSHTWVTVKNGSGDMIFEGNSEKEIPVLNELPVPMVARYIRINPQSWFDNG | 751 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| P1310: | 223 | TSYKVMVSNDSHTWVTVKNGSGDMIFEGNSEKEIPVLNELPVPMVARYIRINPQSWFDNG | 282 | |
| NOV12: | 752 | SICMRMEILGCPLPDPNNYYHRRNEMTTTDDLDFKHHNYKEMRQVQLMKVVNEMCPNITR | 931 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\| | | |
| P1310: | 283 | SICMRMEILGCPLPDPNNYYHRRNEMTTTDDLDFKHHNYKEMRQ--LMKVVNEMCPNITR | 340 | |
| NOV12: | 932 | IYNIGKSHQGLKLYAVEISDHPGEHEVGEPEFHYIAGAHGNEVLGRELLLLLVQFVCQEY | 1111 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| P1310: | 341 | IYNIGKSHQGLKLYAVEISDHPGEHEVGEPEFHYIAGAHGNEVLGRELLLLLVQFVCQEY | 400 | |
| NOV12: | 1112 | LARNARIVHLVEETRIHVLPSLNPDGYEKAYEGGSELGGWSLGRWTHDGIDINNNFPDLN | 1291 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| P1310: | 401 | LARNARIVHLVEETRIHVLPSLNPDGYEKAYEGGSELGGWSLGRWTHDGIDINNNFPDLN | 460 | |
| NOV12: | 1292 | TLLWEAEDRQNVPRKVPNHYIAIPEWFLSENATVVAAETRAVIAWMEKIPFVLGGNLQGG | 1471 | |
| | | \|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\| | | |
| P1310: | 461 | TLLWEAEDRQNVPRKVPNHYIAIPEWFLSENATV-AAETRAVIAWMEKIPFVLGGNLQGG | 519 | |
| NOV12: | 1472 | ELVVAYPYDLVRSPWKTQEHTPTPDDHVFRWLAYSYASTHRLMTDARRRVCHTEDFQKEE | 1651 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| P1310: | 520 | ELVVAYPYDLVRSPWKTQEHTPTPDDHVFRWLAYSYASTHRLMTDARRRVCHTEDFQKEE | 579 | |
| NOV12: | 1652 | GTVNGASWHTVAGSLNDFSYLHTNCFELSIYVGCDKYPHESQLPEEWENNRESLIVFMEQ | 1831 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| P1310: | 580 | GTVNGASWHTVAGSLNDFSYLHTNCFELSIYVGCDKYPHESQLPEEWENNRESLIVFMEQ | 639 | |
| NOV12: | 1832 | VHRGIKGLVRDSHGKGIPNAIISVEGINHDIRTANDGDYWRLLNPGEYVVTAKAEGFTAS | 2011 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| P1310: | 640 | VHRGIKGLVRDSHGKGIPNAIISVEGINHDIRTANDGDYWRLLNPGEYVVTAKAEGFTAS | 699 | |
| NOV12: | 2012 | TKNCMVGYDMGATRCDFTLSKTNMARIREIMEKFGKQPVSLPARRLKLRGRKRRQRG 2182 | | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| P1310: | 700 | TKNCMVGYDMGATRCDFTLSKTNMARIREIMEKFGKQPVSLPARRLKLRGRKRRQRG 756 | | |

Where | indicates identity and + indicates similarity.

Where | indicates identity and + indicates similarity.

TABLE 37

| | | | | |
|---|---|---|---|---|
| NOV12: | 656 | NSEKEIPVLNELPVPMVARYIRINPQSWFDNGSICMRMEILGCPLPDPNNYYHRRNEMTT | 835 | (SEQ ID NO.: 63) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| HLTA: | 1 | NSEKEIPVLNELPVPMVARYIRINPQSWFDNGSICMRMEILGCPLPDPNNYYHRRNEMTT | 60 | (SEQ ID NO.: 64) |
| NOV12: | 836 | TDDLDFKHHNYKEMRQVQLMKVVNEMCPNITRIYNIGKSHQGLKLYAVEISDHPGEHEVG | 1015 | |
| | | \|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\| | | |
| HLTA: | 61 | TDDLDFKHHNYKEMRQ--LMKVVNEMCPNITRIYNIGKSHQGLKLYAVEISDHPGEHEVG | 118 | |
| NOV12: | 1016 | EPEFHYIAGAHGNEVLGRELLLLLVQFVCQEYLARNARIVHLVEETRIHVLPSLNPDGYE | 1195 | |
| | | \|\|\|\|\|\|\|\|\|\|\|\|\|\|\|\|\|\|\|\|\|\|\|\|+ \|+\|\|\|\| \|+\|\|\|\|\| \|\|\|\|\|\|\|\|+\|\|\|\|\|\|\|\|\|\| | | |
| HLTA: | 119 | EPEFHYIAGAHGNEVLGRELLLLLLHFLCQEYSAQNARIVRLVEETRIHILPSLNPDGYE | 178 | |
| NOV12: | 1196 | KAYEGGSELGGWSLGRWTHDGIDINNNFPDLNTLLWEAEDRQNVPRKVPNHYIAIPEWFL | 1375 | |
| | | \|\|\|\|\|\|\|\|\|\|\|\|\|\|\|\|\|\|\|\|\|\|\|\|\|\|\|\|\|\|\|\|+\|\|\|\|\|\|\|+\|\| \|\|\|\|\|\|\|\|\|\|\|\|\|\|\|\| | | |
| HLTA: | 179 | KAYEGGSELGGWSLGRWTHDGIDINNNFPDLNSLLWEAEDQQNAPRKVPNHYIAIPEWFL | 238 | |
| NOV12: | 1376 | SENATVVAAETRAVIAWMEKIPFVLGGNLQGGELVVAYPYDLVRSPWKTQEHTPTPDDHV | 1555 | |
| | | \|\|\|\|\|\| \| \|\|\|\|\|\|\|\|\|\|\|\|\|\|\|\|\|\|\|\|\|\|\|\|\|\|\|\|\|\|\|\|+\|\|\| \|\|\|\|\|\|\|\|\|\|\|\|\|\| | | |
| HLTA: | 239 | SENATV-ATETRAVIAWMEKIPFVLGGNLQGGELVVAYPYDMVRSLWKTQEHTPTPDDHV | 297 | |
| NOV12: | 1556 | FRWLAYSYASTHRLMTDARRRVCHTEDFQKEEGTVNGASWHTVAGSLNDFSYLHTNCFEL | 1735 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| HLTA: | 298 | FRWLAYSYASTHRLMTDARRRVCHTEDFQKEEGTVNGASWHTVAGSLNDFSYLHTNCFEL | 357 | |
| NOV12: | 1736 | SIYVGCDKYPHESQLPEEWENNRESLIVFMEQVHRGIKGLVRDSHGKGIPNAIISVEGIN | 1915 | |
| | | \|\|\|\|\|\|\|\|\|\|\|\|\|+\|\|\|\|\|\|\|\|\|\|\|\|\|\|\|\|\|\|\|\|\|\|\|\|\|+\|\|\| \|\|\|\| \|\|+\|\|\|\|\|+\| | | |
| HLTA: | 358 | SIYVGCDKYPHESELPEEWENNRESLIVFMEQVHRGIKGIVRDLQGKGISNAVISVEGVN | 417 | |
| NOV12: | 1916 | HDIRTANDGDYWRLLNPGEYVVTAKAEGFTASTKNCMVGYDMGATRCDFTLSKTNMARIR | 2095 | |
| | | \|\|\|\|\|\|+\|\|\|\|\|\|\|\|\|\|\|\|\|\|\|\|\|\|\|\|\|\| \|\|\|\|\|\|\|\|\|\|\|\|\|\|\|\|\|\|\|\|+\|\|\|+\|\|\|\| | | |
| HLTA: | 418 | HDIRTASDGDYWRLLNPGEYVVTAKAEGFITSTKNCMVGYDMGATRCDFTLTKTNLARIR | 477 | |
| NOV12: | 2096 | EIMEKFGKQPVSLPARRLKLRGRKRRQRG 2182 | | |
| | | \|\|\|\| \|\|\|\|\|\|\|\|\|+\|\|\|\|\|\|\|\|\|\|\|\|\|\| | | |
| HLTA: | 478 | EIMETFGKQPVSLPSRRLKLRGRKRRQRG 506 | | |

Where | indicates identity and + indicates similarity.

Where | indicates identity and + indicates similarity.

Carboxypeptidase-like proteins are important in cell differentiation. Layne and co-workers found that the aortic carboxypeptidase-like protein, a novel protein with discoidin and carboxypeptidase-like domains, is up-regulated during vascular smooth muscle cell differentiation. Phenotypic modulation of vascular smooth muscle cells plays an important role in the pathogenesis of arteriosclerosis. In a screen of proteins expressed in human aortic smooth muscle cells, they identified a novel gene product designated aortic carboxypeptidase-like protein (ACLP). The approximately 4-kilobase human cDNA and its mouse homologue encode 1158 and 1128 amino acid proteins, respectively, that are 85% identical. ACLP is a nonnuclear protein that contains a signal peptide, a lysine- and proline-rich 11-amino acid repeating motif, a discoidin-like domain, and a C-terminal domain with 39% identity to carboxypeptidase E. By Western blot analysis and in situ hybridization, Layne et al. detected abundant ACLP expression in the adult aorta. ACLP was expressed predominantly in the smooth muscle cells of the adult mouse aorta but not in the adventitia or in several other tissues. In cultured mouse aortic smooth muscle cells, ACLP mRNA and protein were up-regulated 2-3-fold after serum starvation. Using a recently developed neural crest cell to smooth muscle cell in vitro differentiation system, Layne and co-workers found that ACLP mRNA and protein were not expressed in neural crest cells but were up-regulated dramatically with the differentiation of these cells. These results indicate that ACLP may play a role in differentiated vascular smooth muscle cells (See Layne et al., 1998, J Biol Chem 273:15654).

NOV12 represents a new member of the carboxypeptidase family of proteins. NOV12 is useful in determining changes in expression of genes contained within the carboxypeptidase protein family. NOV12 satisfies a need in the art by providing new diagnostic or therapeutic compositions useful in the treatment of disorders associated with alterations in the expression of members of the carboxypeptidase-associated protein family of proteins. NOV12 nucleic acids, polypeptides, antibodies, and other compositions of the present invention are useful in the treatment and/or diagnosis of a variety of diseases and pathologies, including by way of nonlimiting example, those involving disorders of vascular smooth muscle cell differentiation, e.g. heart failure, atherosclerosis, hypertension and stroke.

The nucleic acids and proteins of the invention are useful in potential therapeutic applications implicated in disorders characterized by aberrant cell proliferation, differentiation and migration, e.g. cancer, angiogenesis, atherosclerosis and obesity, neurological disorders, e.g. stroke, Pendred syndrome, multiple sclerosis and Alzheimer's disease, keratinocyte defects, e.g. lesional psoriatic skin, ischemic disorders, e.g. diabetic retinopathy, hepatic disorders, e.g. cirrhotic hepatitis, and pancreatic disorders e.g. acute pancreatitis. For example, a cDNA encoding a sulfate anion transporter-like protein may be useful in gene therapy for treating Pendred syndrome and other such disorders, and the sulfate anion transporter-like protein may be useful when administered to a subject in need thereof. By way of nonlimiting example, the compositions of the present invention will have efficacy for treatment of patients suffering from disorders of the ion regulatory system. The novel nucleic acids encoding a chloride channel-like protein, and the chloride channel-like protein of the invention, or fragments thereof, may further be useful in the treatment of cystic fibrosis, Dent's disease, Bartter's syndrome and Gittelman's syndrome, development of powerful assay systems for functional analysis of various human disorders which will help in understanding of pathology of the disease, and development of new drug targets for various disorders. They may also be used in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

NOVX Nucleic Acids

The nucleic acids of the invention include those that encode a NOVX polypeptide or protein. As used herein, the terms polypeptide and protein are interchangeable.

In some embodiments, a NOVX nucleic acid encodes a mature NOVX polypeptide. As used herein, a “mature” form of a polypeptide or protein described herein relates to the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an open reading frame described herein. The product “mature” form arises, again by way of nonlimiting example, as a result of one or more naturally occurring processing steps that may take place within the cell in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

Among the NOVX nucleic acids is the nucleic acid whose sequence is provided in SEQ D) NO: 1, 3, 5, 7, 9, 11, 13, 15, 17, 19, 21 or 23, or a fragment thereof. Additionally, the invention includes mutant or variant nucleic acids of SEQ ID NO: 1, 3, 5, 7, 9, 11, 13, 15, 17, 19, 21 or 23, or a fragment thereof, any of whose bases may be changed from the corresponding bases shown in SEQ ID NO: 1, 3, 5, 7, 9, 11, 13, 15, 17, 19, 21 or 23, while still encoding a protein that maintains at least one of its NOVX-like activities and physiological functions (i.e., modulating angiogenesis, neuronal development). The invention further includes the complement of the nucleic acid sequence of SEQ ID NO: 1, 3, 5, 7, 9, 11, 13, 15, 17, 19, 21 or 23, including fragments, derivatives, analogs and homologs thereof. The invention additionally includes nucleic acids or nucleic acid fragments, or complements thereto, whose structures include chemical modifications.

One aspect of the invention pertains to isolated nucleic acid molecules that encode NOVX proteins or biologically active portions thereof. Also included are nucleic acid fragments sufficient for use as hybridization probes to identify NOVX-encoding nucleic acids (e.g., NOVX mRNA) and fragments for use as polymerase chain reaction (PCR) primers for the amplification or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt), 100 nt, or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules that are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated NOVX nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of SEQ ID NO: 1, 3, 5, 7, 9, 11, 13, 15, 17, 19, 21 or 23, or a complement of any of this nucleotide sequence, can be isolated using standard molecular biology techniques and the sequence information provided herein. Using all or a portion of the nucleic acid sequence of SEQ ID NO: 1, 3, 5, 7, 9, 11, 13, 15, 17, 19, 21 or 23, as a hybridization probe, NOVX nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.) A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at lease 6 contiguous nucleotides of SEQ ID NO: 1, 3, 5, 7, 9, 11, 13, 15, 17, 19, 21 or 23, or a complement thereof. Oligonucleotides may be chemically synthesized and may be used as probes.

In another embodiment, an isolated nucleic acid molecule of the invention comprises a nucleic acid molecule that is a complement of the nucleotide sequence shown in SEQ ID NO: 1, 3, 5, 7, 9, 11, 13, 15, 17, 19, 21 or 23, or a portion of this nucleotide sequence. A nucleic acid molecule that is complementary to the nucleotide sequence shown in SEQ ID NO: 1, 3, 5, 7, 9, 11, 13, 15, 17, 19, 21 or 23 is one that is sufficiently complementary to the nucleotide sequence shown in SEQ ID NO: 1, 3, 5, 7, 9, 11, 13, 15, 17, 19, 21 or 23 that it can hydrogen bond with little or no mismatches to the nucleotide sequence shown in SEQ ID NO: 1, 3, 5, 7, 9, 11, 13, 15, 17, 19, 21 or 23, thereby forming a stable duplex.

As used herein, the term “complementary” refers to Watson-Crick or Hoogsteen base pairing between nucleotide units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of SEQ ID NO: 1, 3, 5, 7, 9, 11, 13, 15, 17, 19, 21 or 23, e.g., a fragment that can be used as a probe or primer, or a fragment encoding a biologically active portion of NOVX. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, 85%, 90%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489, which is incorporated herein by reference in its entirety).

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of a NOVX polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a NOVX polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding huma NOVX protein. Homologous nucleic acid sequences include those nucleic acid sequences that encode conservative amino acid substitutions (see below) SEQ ID NO: 2, 4, 6, 8, 10, 12, 14, 16, 18, 20, 22 or 24, as well as a polypeptide having NOVX activity. Biological activities of the NOVX proteins are described below. A homologous amino acid sequence does not encode the amino acid sequence of a huma NOVX polypeptide.

The nucleotide sequence determined from the cloning of the huma NOVX gene allows for the generation of probes and primers designed for use in identifying and/or cloning NOVX homologues in other cell types, e.g., from other tissues, as well as NOVX homologues from other mammals. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 or more consecutive sense strand nucleotide sequence of SEQ ID NO: 1, 3, 5, 7, 9, 11, 13, 15, 17, 19, 21 or 23; or an anti-sense strand nucleotide sequence of SEQ ID NO: 1, 3, 5, 7, 9, 11, 13, 15, 17, 19, 21 or 23; or of a naturally occurring mutant of SEQ ID NO: 1, 3, 5, 7, 9, 11, 13, 15, 17, 19, 21 or 23.

Probes based on the huma NOVX nucleotide sequence can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a NOVX protein, such as by measuring a level of a NOVX-encoding nucleic acid in a sample of cells from a subject e.g., detecting NOVX mRNA levels or determining whether a genomic NOVX gene has been mutated or deleted.

A “polypeptide having a biologically active portion of NOVX” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of NOVX” can be prepared by isolating a portion of SEQ ID NO: 1, 3, 5, 7, 9, 11, 13, 15, 17, 19, 21 or 23 that encodes a polypeptide having a NOVX biological activity (biological activities of the NOVX proteins are described below), expressing the encoded portion of NOVX protein (e.g., by recombinant expression in vitro) and assessing the activity of the encoded portion of NOVX. For example, a nucleic acid fragment encoding a biologically active portion of NOVX can optionally include an ATP-binding domain. In another embodiment, a nucleic acid fragment encoding a biologically active portion of NOVX includes one or more regions.

NOVX Variants

The invention further encompasses nucleic acid molecules that differ from the nucleotide sequences shown in SEQ ID NO: 1, 3, 5, 7, 9, 11, 13, 15, 17, 19, 21 or 23 due to the degeneracy of the genetic code. These nucleic acids thus encode the same NOVX protein as that encoded by the nucleotide sequence shown in SEQ ID NO: 1, 3, 5, 7, 9, 11, 13, 15, 17, 19, 21 or 23 e.g., the polypeptide of SEQ ID NO: 2, 4, 6, 8, 10, 12, 14, 16, 18, 20, 22 or 24. In another embodiment, an isolated nucleic acid molecule of the invention has a nucleotide sequence encoding a protein having an amino acid sequence shown SEQ ID NO: 2, 4, 6, 8, 10, 12, 14, 16, 18, 20, 22 or 24.

In addition to the huma NOVX nucleotide sequence shown in SEQ ID NO: 1, 3, 5, 7, 9, 11, 13, 15, 17, 19, 21 or 23, it will be appreciated by those skilled in the art that DNA sequence polymorphisms that lead to changes in the amino acid sequences of NOVX may exist within a population (e.g., the human population). Such genetic polymorphism in the NOVX gene may exist among individuals within a population due to natural allelic variation. As used herein, the terms “gene” and “recombinant gene” refer to nucleic acid molecules comprising an open reading frame encoding a NOVX protein, preferably a mammalia NOVX protein. Such natural allelic variations can typically result in 1-5% variance in the nucleotide sequence of the NOVX gene. Any and all such nucleotide variations and resulting amino acid polymorphisms in NOVX that are the result of natural allelic variation and that do not alter the functional activity of NOVX are intended to be within the scope of the invention.

Moreover, nucleic acid molecules encoding NOVX proteins from other species, and thus that have a nucleotide sequence that differs from the human sequence of SEQ ID NO: 1, 3, 5, 7, 9, 11, 13, 15, 17, 19, 21 or 23 are intended to be within the scope of the invention. Nucleic acid molecules corresponding to natural allelic variants and homologues of the NOVX cDNAs of the invention can be isolated based on their homology to the huma NOVX nucleic acids disclosed herein using the human cDNAs, or a portion thereof, as a hybridization probe according to standard hybridization techniques under stringent hybridization conditions. For example, a soluble huma NOVX cDNA can be isolated based on its homology to human membrane-bound NOVX. Likewise, a membrane-bound huma NOVX cDNA can be isolated based on its homology to soluble huma NOVX.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO: 1, 3, 5, 7, 9, 11, 13, 15, 17, 19, 21 or 23. In another embodiment, the nucleic acid is at least 10, 25, 50, 100, 250, 500 or 750 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tₘ) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of SEQ ID NO: 1, 3, 5, 7, 9, 11, 13, 15, 17, 19, 21 or 23 corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO: 1, 3, 5, 7, 9, 11, 13, 15, 17, 19, 21 or 23, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of SEQ ID NO: 1, 3, 5, 7, 9, 11, 13, 15, 17, 19, 21 or 23, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981, Proc Natl Acad Sci USA 78: 6789-6792.

Conservative Mutations

In addition to naturally-occurring allelic variants of the NOVX sequence that may exist in the population, the skilled artisan will further appreciate that changes can be introduced by mutation into the nucleotide sequence of SEQ ID NO: 1, 3, 5, 7, 9, 11, 13, 15, 17, 19, 21 or 23, thereby leading to changes in the amino acid sequence of the encoded NOVX protein, without altering the functional ability of the NOVX protein. For example, nucleotide substitutions leading to amino acid substitutions at “non-essential” amino acid residues can be made in the sequence of SEQ ID NO: 1, 3, 5, 7, 9, 11, 13, 15, 17, 19, 21 or 23. A “non-essential” amino acid residue is a residue that can be altered from the wild-type sequence of NOVX without altering the biological activity, whereas an “essential” amino acid residue is required for biological activity. For example, amino acid residues that are conserved among the NOVX proteins of the present invention, are predicted to be particularly unamenable to alteration.

Another aspect of the invention pertains to nucleic acid molecules encoding NOVX proteins that contain changes in amino acid residues that are not essential for activity. Such NOVX proteins differ in amino acid sequence from SEQ ID NO: 2, 4, 6, 8, 10, 12, 14, 16, 18, 20, 22 or 24, yet retain biological activity. In one embodiment, the isolated nucleic acid molecule comprises a nucleotide sequence encoding a protein, wherein the protein comprises an amino acid sequence at least about 75% homologous to the amino acid sequence of SEQ ID NO: 2, 4, 6, 8, 10, 12, 14, 16, 18, 20, 22 or 24. Preferably, the protein encoded by the nucleic acid is at least about 80% homologous to SEQ ID NO: 2, 4, 6, 8, 10, 12, 14, 16, 18, 20, 22 or 24, more preferably at least about 90%, 95%, 98%, and most preferably at least about 99% homologous to SEQ ID NO: 2, 4, 6, 8, 10, 12, 14, 16, 18, 20, 22 or 24.

An isolated nucleic acid molecule encoding a NOVX protein homologous to the protein of can be created by introducing one or more nucleotide substitutions, additions or deletions into the nucleotide sequence of SEQ ID NO: 1, 3, 5, 7, 9, 11, 13, 15, 17, 19, 21 or 23, such that one or more amino acid substitutions, additions or deletions are introduced into the encoded protein.

Mutations can be introduced into the nucleotide sequence of SEQ ID NO: 1, 3, 5, 7, 9, 11, 13, 15, 17, 19, 21 or 23 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in NOVX is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of SEQ ID NO: 1, 3, 5, 7, 9, 11, 13, 15, 17, 19, 21 or 23 the encoded protein can be expressed by any recombinant technology known in the art and the activity of the protein can be determined.

In one embodiment, a mutant NOVX protein can be assayed for (1) the ability to form protein:protein interactions with other NOVX proteins, other cell-surface proteins, or biologically active portions thereof, (2) complex formation between a mutant NOVX protein and a NOVX receptor; (3) the ability of a mutant NOVX protein to bind to an intracellular target protein or biologically active portion thereof; (e.g., avidin proteins); (4) the ability to bind NOVX protein; or (5) the ability to specifically bind an anti-NOVX protein antibody.

Antisense NOVX Nucleic Acids

Another aspect of the invention pertains to isolated antisense nucleic acid molecules that are hybridizable to or complementary to the nucleic acid molecule comprising the nucleotide sequence of SEQ ID NO: 1, 3, 5, 7, 9, 11, 13, 15, 17, 19, 21 or 23, or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO: 2, 4, 6, 8, 10, 12, 14, 16, 18, 20, 22 or 24 or antisense nucleic acids complementary to a NOVX nucleic acid sequence of SEQ ID NO: 1, 3, 5, 7, 9, 11, 13, 15, 17, 19, 21 or 23 are additionally provided.

In one embodiment, an antisense nucleic acid molecule is antisense to a “coding region” of the coding strand of a nucleotide sequence encoding NOVX. The term “coding region” refers to the region of the nucleotide sequence comprising codons which are translated into amino acid residues (e.g., the protein coding region of huma NOVX corresponds to SEQ ID NO: 2, 4, 6, 8, 10, 12, 14, 16, 18, 20, 22 or 24). In another embodiment, the antisense nucleic acid molecule is antisense to a “noncoding region” of the coding strand of a nucleotide sequence encoding NOVX. The term “noncoding region” refers to 5′ and 3′ sequences which flank the coding region that are not translated into amino acids (i.e., also referred to as 5′ and 3′ untranslated regions).

Given the coding strand sequences encoding NOVX disclosed herein (e.g., SEQ ID NO: 1, 3, 5, 7, 9, 11, 13, 15, 17, 19, 21 or 23), antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA -DNA analogue (Inoue et al. (1987) FEBS Lett 215: 327-330).

Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

NOVX Ribozymes and PNA Moieties

In still another embodiment, an antisense nucleic acid of the invention is a ribozyme. Ribozymes are catalytic RNA molecules with ribonuclease activity that are capable of cleaving a single-stranded nucleic acid, such as a mRNA, to which they have a complementary region. Thus, ribozymes (e.g., hammerhead ribozymes (described in Haselhoff and Gerlach (1988) Nature 334:585-591)) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX DNA disclosed herein (i.e., SEQ ID NO: 1, 3, 5, 7, 9, 11, 13, 15, 17, 19, 21 or 23). For example, a derivative of a Tetrahymena L-19 IVS RNA can be constructed in which the nucleotide sequence of the active site is complementary to the nucleotide sequence to be cleaved in a NOVX-encoding mRNA. See, e.g., Cech et al. U.S. Pat. No. 4,987,071; and Cech et al. U.S. Pat. No. 5,116,742. Alternatively, NOVX mRNA can be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX (e.g., the NOVX promoter and/or enhancers) to form triple helical structures that prevent transcription of the NOVX gene in target cells. See generally, Helene. (1991) Anticancer Drug Des. 6: 569-84; Helene. et al. (1992) Ann. N.Y. Acad. Sci. 660:27-36; and Maher (1992) Bioassays 14: 807-15.

In various embodiments, the nucleic acids of NOVX can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl) amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810) or the blood-brain barrier (see, e.g., PCT Publication No. WO89/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

NOVX Polypeptides

A NOVX polypeptide of the invention includes the NOVX-like protein whose sequence is provided SEQ ID NO: 2, 4, 6, 8, 10, 12, 14, 16, 18, 20, 22 or 24. The invention also includes a mutant or variant protein any of whose residues may be changed from the corresponding residue shown SEQ ID NO: 2, 4, 6, 8, 10, 12, 14, 16, 18, 20, 22 or 24 while still encoding a protein that maintains its NOVX-like activities and physiological functions, or a functional fragment thereof. In some embodiments, up to 20% or more of the residues may be so changed in the mutant or variant protein. In some embodiments, the NOVX polypeptide according to the invention is a mature polypeptide.

In general, a NOVX-like variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” protein or biologically active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX protein in which the protein is separated from cellular components of the cells from which it is isolated or recombinantly produced. In one embodiment, the language “substantially free of cellular material” includes preparations of NOVX protein having less than about 30% (by dry weight) of non-NOVX protein (also referred to herein as a “contaminating protein”), more preferably less than about 20% of non-NOVX protein, still more preferably less than about 10% of non-NOVX protein, and most preferably less than about 5% non-NOVX protein. When the NOVX protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of NOVX protein in which the protein is separated from chemical precursors or other chemicals that are involved in the synthesis of the protein. In one embodiment, the language “substantially free of chemical precursors or other chemicals” includes preparations of NOVX protein having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically active portions of a NOVX protein include peptides comprising amino acid sequences sufficiently homologous to or derived from the amino acid sequence of the NOVX protein, e.g., the amino acid sequence shown SEQ ID NO: 2, 4, 6, 8, 10, 12, 14, 16, 18, 20, 22 or 24 that include fewer amino acids than the full length NOVX proteins, and exhibit at least one activity of a NOVX protein. Typically, biologically active portions comprise a domain or motif with at least one activity of the NOVX protein. A biologically active portion of a NOVX protein can be a polypeptide which is, for example, 10, 25, 50, 100 or more amino acids in length.

A biologically active portion of a NOVX protein of the present invention may contain at least one of the above-identified domains conserved between the NOVX proteins, e.g. TSR modules. Moreover, other biologically active portions, in which other regions of the protein are deleted, can be prepared by recombinant techniques and evaluated for one or more of the functional activities of a native NOVX protein.

In an embodiment, the NOVX protein has an amino acid sequence shown SEQ ID NO: 2, 4, 6, 8, 10, 12, 14, 16, 18, 20, 22 or 24. In other embodiments, the NOVX protein is substantially homologous to SEQ ID NO: 2, 4, 6, 8, 10, 12, 14, 16, 18, 20, 22 or 24 and retains the functional activity of the protein of SEQ ID NO: 2, 4, 6, 8, 10, 12, 14, 16, 18, 20, 22 or 24 yet differs in amino acid sequence due to natural allelic variation or mutagenesis, as described in detail below. Accordingly, in another embodiment, the NOVX protein is a protein that comprises an amino acid sequence at least about 45% homologous to the amino acid sequence of SEQ ID NO: 2, 4, 6, 8, 10, 12, 14, 16, 18, 20, 22 or 24 and retains the functional activity of the NOVX proteins of SEQ ID NO: 2, 4, 6, 8, 10, 12, 14, 16, 18, 20, 22 or 24.

Determining Homology between Two or More Sequences

To determine the percent homology of two amino acid sequences or of two nucleic acids, the sequences are aligned for optimal comparison purposes (e.g., gaps can be introduced in either of the sequences being compared for optimal alignment between the sequences). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch 1970 J Mol Biol 48: 443453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO: 1, 3, 5, 7, 9, 11, 13, 15, 17, 19, 21 or 23.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region. The term “percentage of positive residues” is calculated by comparing two optimally aligned sequences over that region of comparison, determining the number of positions at which the identical and conservative amino acid substitutions, as defined above, occur in both sequences to yield the number of matched positions, dividing the number of matched positions by the total number of positions in the region of comparison (i.e., the window size), and multiplying the result by 100 to yield the percentage of positive residues.

Chimeric and Fusion Proteins

The invention also provides NOVX chimeric or fusion proteins. As used herein, a NOVX “chimeric protein” or “fusion protein” comprises a NOVX polypeptide operatively linked to a non-NOVX polypeptide. An “NOVX polypeptide” refers to a polypeptide having an amino acid sequence corresponding to NOVX, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically active portion of a NOVX protein. In another embodiment, a NOVX fusion protein comprises at least two biologically active portions of a NOVX protein. Within the fusion protein, the term “operatively linked” is intended to indicate that the NOVX polypeptide and the non-NOVX polypeptide are fused in-frame to each other. The non-NOVX polypeptide can be fused to the N-terminus or C-terminus of the NOVX polypeptide.

For example, in one embodiment a NOVX fusion protein comprises a NOVX polypeptide operably linked to the extracellular domain of a second protein. Such fusion proteins can be further utilized in screening assays for compounds that modulate NOVX activity (such assays are described in detail below).

In another embodiment, the fusion protein is a GST-NOVX fusion protein in which the NOVX sequences are fused to the C-terminus of the GST (i.e., glutathione S-transferase) sequences. Such fusion proteins can facilitate the purification of recombinant NOVX.

In another embodiment, the fusion protein is a NOVX-immunoglobulin fusion protein in which the NOVX sequences comprising one or more domains are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. In one nonlimiting example, a contemplated NOVX ligand of the invention is the NOVX receptor. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, e,g., cancer as well as modulating (e.g., promoting or inhibiting) cell survival, as well as acute and chronic inflammatory disorders and hyperplastic wound healing, e.g. hypertrophic scars and keloids. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX Agonists and Antagonists

The present invention also pertains to variants of the NOVX proteins that function as either NOVX agonists (mimetics) or as NOVX antagonists. Variants of the NOVX protein can be generated by mutagenesis, e.g., discrete point mutation or truncation of the NOVX protein. An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX protein that function as either NOVX agonists (mimetics) or as NOVX antagonists can be identified by screening combinatorial libraries of mutants, e.g., truncation mutants, of the NOVX protein for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl Acid Res 11:477.

Polypeptide Libraries

In addition, libraries of fragments of the NOVX protein coding sequence can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the NOVX protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique that enhances the frequency of functional mutants in the libraries, can be used in combination with the screening assays to identify NOVX variants (Arkin and Yourvan (1992) PNAS 89:7811-7815; Delgrave et al. (1993) Protein Engineering 6:327-331).

NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence shown in from SEQ ID NO: 2, 4, 6, 8, 10, 12, 14, 16, 18, 20, 22 or 24, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the huma NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown iv vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and Fc{cube root}RIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 3140), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as human, Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. Sequences including SEQ ID NO: 1, 3, 5, 7, 9, 11, 13, 15, 17, 19, 21 or 23 can be introduced as a transgene into the genome of a non-human animal. Alternatively, a non-human homologue of the huma NOVX gene, such as a mouse NOVX gene, can be isolated based on hybridization to the huma NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the DNA of SEQ ID NO: 1, 3, 5, 7, 9, 11, 13, 15, 17, 19, 21 or 23), but more preferably, is a non-human homologue of a huma NOVX gene. For example, a mouse homologue of huma NOVX gene of SEQ ID NO: 1, 3, 5, 7, 9, 11, 13, 15, 17, 19, 21 or 23 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York. If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., 1993 Proc. Natl. Acad. Sci. USA, 90: 7889-7893. The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended. The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for iv vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein. In addition, the anti-NOVX antibodies of the invention can be used to detect and isolate NOVX proteins and modulate NOVX activity. For example, NOVX activity includes growth and differentiation, antibody production, and tumor growth.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to a NOVX protein determined. The cell, for example, can be of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with a NOVX target molecule. As used herein, a “target molecule” is a molecule with which a NOVX protein binds or interacts in nature, for example, a molecule on the surface of a cell which expresses a NOVX interacting protein, a molecule on the surface of a second cell, a molecule in the extracellular milieu, a molecule associated with the internal surface of a cell membrane or a cytoplasmic molecule. A NOVX target molecule can be a non-NOVX molecule or a NOVX protein or polypeptide of the invention In one embodiment, a NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct binding. In an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate substrate can be determined as described above.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl—N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) identify an individual from a minute biological sample (tissue typing); and (ii) aid in forensic identification of a biological sample. Some of these applications are described in the subsections, below.

Tissue Typing

The NOVX sequences of the invention can be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NO: 1, 3, 5, 7, 9, 11, 13, 15, 17, 19, 21 or 23 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. Disorders associated with aberrant NOVX expression of activity include, for example, disorders characterized by aberrant cell proliferation, differentiation and migration, e.g. cancer, angiogenesis, atherosclerosis and obesity, neurological disorders, e.g. stroke, Pendred syndrome, multiple sclerosis and Alzheimer's disease, keratinocyte defects, e.g. lesional psoriatic skin, ischemic disorders, e.g. diabetic retinopathy, hepatic disorders, e.g. cirrhotic hepatitis, and pancreatic disorders e.g. acute pancreatitis.

The invention also provides for prognostic (or predictive) assays for determining whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.) Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO: 1, 3, 5, 7, 9, 11, 13, 15, 17, 19, 21 or 23, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

One agent for detecting NOVX protein is an antibody capable of binding to NOVX protein, preferably an antibody with a detectable label. Antibodies directed against a protein of the invention may be used in methods known within the art relating to the localization and/or quantitation of the protein (e.g., for use in measuring levels of the protein within appropriate physiological samples, for use in diagnostic methods, for use in imaging the protein, and the like). In a given embodiment, antibodies against the proteins, or derivatives, fragments, analogs or homologs thereof, that contain the antigen binding domain, are utilized as pharmacologically-active compounds.

An antibody specific for a protein of the invention can be used to isolate the protein by standard techniques, such as immunoaffinity chromatography or immunoprecipitation. Such an antibody can facilitate the purification of the natural protein antigen from cells and of recombinantly produced antigen expressed in host cells. Moreover, such an antibody can be used to detect the antigenic protein (e.g., in a cellular lysate or cell supernatant) in order to evaluate the abundance and pattern of expression of the antigenic protein. Antibodies directed against the protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In one embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Such disorders include for example, disorders characterized by aberrant cell proliferation, differentiation and migration, e.g. cancer, angiogenesis, atherosclerosis and obesity, neurological disorders, e.g. stroke, Pendred syndrome, multiple sclerosis and Alzheimer's disease, keratinocyte defects, e.g. lesional psoriatic skin, ischemic disorders, e.g. diabetic retinopathy, hepatic disorders, e.g. cirrhotic hepatitis, and pancreatic disorders e.g. acute pancreatitis.

Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7:5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders characterized by aberrant cell proliferation, differentiation and migration, e.g. cancer, angiogenesis, atherosclerosis and obesity, neurological disorders, e.g. stroke, Pendred syndrome, multiple sclerosis and Alzheimer's disease, keratinocyte defects, e.g. lesional psoriatic skin, ischemic disorders, e.g. diabetic retinopathy, hepatic disorders, e.g. cirrhotic hepatitis, and pancreatic disorders e.g. acute pancreatitis. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. Disorders associated with aberrant NOVX expression include, for example, disorders characterized by aberrant cell proliferation, differentiation and migration, e.g. cancer, angiogenesis, atherosclerosis and obesity, neurological disorders, e.g. stroke, Pendred syndrome, multiple sclerosis and Alzheimer's disease, keratinocyte defects, e.g. lesional psoriatic skin, ischemic disorders, e.g. diabetic retinopathy, hepatic disorders, e.g. cirrhotic hepatitis, and pancreatic disorders e.g. acute pancreatitis. These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated ). Another example of such a situation is where the subject has an immunodeficiency disease (e.g., AIDS).

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

The invention will be further described in the following examples, which do not limit the scope of the invention described in the claims.

### EXAMPLES

### Example 1

### Method of Identifying the Nucleic Acids of the Present Invention

Novel nucleic acid sequences were identified by TblastN using CuraGen Corporation's sequence file run against the Genomic Daily Files made available by GenBank. The nucleic acids were further predicted by the program GenScan™, including selection of exons. These were further modified by means of similarities using BLAST searches. The sequences were then manually corrected for apparent inconsistencies, thereby obtaining the sequences encoding the full-length protein.

### Example 2

### Method of Cloning a NOV11 (CG54656-05) Nucleic Acid

The sequence of NOV11 (Acc. No. CG54656-05) was derived by laboratory cloning of cDNA fragments, by in silico prediction of the sequence. cDNA fragments covering either the full length of the DNA sequence, or part of the sequence, or both, were cloned. In silico prediction was based on sequences available in Curagen's proprietary sequence databases or in the public human sequence databases, and provided either the full length DNA sequence, or some portion thereof.

The laboratory cloning was performed using one or more of the methods summarized below:

SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Exon Linking: The cDNA coding for the CG54656-05 sequence was cloned by the polymerase chain reaction (PCR) using the primers:

| | |
|---|---|
| CAGCTCGCTGTCTTGGTGGTC | (SEQ ID NO.: 64) |
| and | |
| TCACAGGATGATGACACAAGCTCC. | (SEQ ID NO.: 65) |

Primers were designed based on in silico predictions of the full length or some portion (one or more exons) of the cDNA/protein sequence of the invention. These primers were used to amplify a cDNA from a pool containing expressed human sequences derived from the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus.

Multiple clones were sequenced and these fragments were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: The PCR product derived by exon linking, covering the entire open reading frame, was cloned into the pCR2.1 vector from Invitrogen to provide clone GM38019075_A.698002.B7.

### Example 3

### Expression profiling of NOV3 (CG53063-01 or 94115520_EXT)

Panel 1.3 (Table 38): The profile was generated from a panel of 37 normal human tissues and 59 human cancer cell lines using specific gene probe and primer sets (Ag809). This gene is highly expressed in normal fetal heart and adult spleen and to a lesser extent in normal testes, prostate, ovary, mammary gland, trachea stomach, colorectal tissue, brain, pituitary gland and salivary gland.

Panel 4D (1 able 39): The profile was generated from a panel of several human cell lines that were either untreated or treated with a wide variety factors which modulate the immune response. This panel shows that the normal colon expresses high levels of this transcript whereas three different inflammatory bowel disease tissues did not.

| | |
|---|---|
| Probe Name: Ag809 | |
| Forward | |
| 5′-ATGTGATCTTTGGCTGTGAAGT-3′ | (SEQ ID NO.: 66) |
| Probe | |
| FAM-5′-CTACCCCATGGCCTCCATCGAGT-3′- | (SEQ ID NO.: 67) |
| TAMRA | |
| Reverse | |
| 5′-GGATGTCCAAGCCATCCTT-3′ | (SEQ ID NO.: 68) |

TABLE 38

| panel 1.3 ag809 | 1.3Dtm3313f_ag809 |
|---|---|
| Adipose | 1.14 |
| Adrenal gland | 0.45 |
| Bladder | 0.72 |
| Bone marrow | 0.7 |
| Brain (amygdala) | 0.46 |
| Brain (cerebellum) | 0 |
| Brain (fetal) | 0 |
| Brain (hippocampus) | 2.52 |
| Cerebral Cortex | 0.44 |
| Brain (substantia nigra) | 0.16 |
| Brain (thalamus) | 0.58 |
| Brain (whole) | 0.58 |
| Colorectal | 2.3 |
| Heart (fetal) | 8.48 |
| Liver adenocarcinoma | 15.39 |
| Heart | 2.68 |
| Kidney | 0.34 |
| Kidney (fetal) | 0.36 |
| Liver | 0.16 |
| Liver (fetal) | 0.21 |
| Lung | 0.75 |
| Lung (fetal) | 1.73 |
| Lymph node | 0.64 |
| Mammary gland | 1.92 |
| Fetal Skeletal | 28.32 |
| Ovary | 2.34 |
| Pancreas | 0.41 |
| Pituitary gland | 1.69 |
| Plancenta | 0.9 |
| Prostate | 3.04 |
| Salivary gland | 2.38 |
| Skeletal muscle | 0.63 |
| Small intestine | 0.88 |
| Spinal cord | 0.51 |
| Spleen | 11.91 |
| Stomach | 5.48 |
| Testis | 2.82 |
| Thymus | 0.81 |
| Thyroid | 1.25 |
| Trachea | 4.54 |
| Uterus | 1.33 |
| Breast ca.* (pl. effusion) MCF-7 | 1.09 |
| Breast ca.* (pl. ef) MDA-MB-231 | 4.12 |
| Breast ca. BT-549 | 1.48 |
| Breast ca.* (pl. effusion) T47D | 12.76 |
| Breast ca. MDA-N | 3.61 |
| Ovarian ca. OVCAR-3 | 0.68 |
| Ovarian ca.* (ascites) SK-OV-3 | 0.08 |
| Ovarian ca. OVCAR-4 | 0.78 |
| Ovarian ca. OVCAR-5 | 1.76 |
| Ovarian ca. IGROV-1 | 1.9 |
| Ovarian ca. OVCAR-8 | 2.45 |
| CNS ca. (glio/astro) U87-MG | 2.34 |
| CNS ca. (astro) SW1783 | 1.26 |
| CNS ca. (glio/astro) U-118-MG | 19.34 |
| CNS ca.* (neuro; met) SK-N-AS | 10.08 |
| CNS ca. (astro) SF-539 | 2.43 |
| CNS ca. (astro) SNB-75 | 2.3 |
| CNS ca. (glio) SNB-19 | 0 |
| CNS ca. (glio) U251 | 0.17 |
| CNS ca. (glio) SF-295 | 2.43 |
| Colon ca. SW480 | 9.02 |
| Colon ca.* (SW480 met) SW620 | 3.67 |
| Colon ca. HT29 | 1.02 |
| Colon ca. HCT-116 | 0.97 |
| Colon ca. CaCo-2 | 9.54 |
| Gastric ca.* (liver met) NCI-N87 | 2.03 |
| 83219 CC Well to Mod Diff (ODO3866) | 0.58 |
| Colon ca. HCC-2998 | 5.37 |
| Renal ca. 786-0 | 1.35 |
| Renal ca. A498 | 1.32 |
| Renal ca. RXF 393 | 0.9 |
| Renal ca. ACHN | 1.9 |
| Renal ca. UO-31 | 0.25 |
| Renal ca. TK-10 | 0.11 |
| Liver ca. (hepatoblast) HepG2 | 10.44 |
| Lung ca. (small cell) LX-1 | 6.65 |
| Lung ca. (small cell) NCI-H69 | 0.57 |
| Lung ca. (s. cell var.) SHP-77 | 2.52 |
| Lung ca. (non-sm. cell) A549 | 0.67 |
| Lung ca. (squam.) SW 900 | 0.91 |
| Lung ca. (squam.) NCI-H596 | 0.13 |
| Lung ca. (non-s. cell) NCI-H23 | 6.65 |
| Lung ca. (large cell) NCI-H460 | 1.32 |
| Lung ca (non-s. cell) HOP-62 | 2.03 |
| Lung ca. (non-s. cl) NCI-H522 | 9.21 |
| Pancreatic ca. CAPAN 2 | 0.9 |
| Prostate ca.* (bone met) PC-3 | 6.93 |
| Melanoma Hs688(A).T | 2.61 |
| Melanoma* (met) Hs688(B).T | 13.77 |
| Melanoma UACC-62 | 0.33 |
| Melanoma M14 | 1.83 |
| Melanoma LOX IMVI | 0.46 |
| Melanoma* (met) SK-MEL-5 | 0.65 |
| genomic DNA control | 100 |
| Chemistry Control | 96.59 |

TABLE 39

| panel 4D ag809 | 4Dtm3315f_ag809 |
|---|---|
| 93768_Secondary Th1_anti-CD28/anti-CD3 | 2.01 |
| 93769_Secondary Th2_anti-CD28/anti-CD3 | 1.5 |
| 93770_Secondary Tr1_anti-CD28/anti-CD3 | 2.45 |
| 93573_Secondary Th1_resting day 4-6 in IL-2 | 0.99 |
| 93572_Secondary Th2_resting day 4-6 in IL-2 | 2.96 |
| 93571_Secondary Tr1_resting day 4-6 in IL-2 | 1.69 |
| 93568_primary Th1_anti-CD28/anti-CD3 | 0.41 |
| 93569_primary Th2_anti-CD28/anti-CD3 | 1.47 |
| 93570_primary Tr1_anti-CD28/anti-CD3 | 1.96 |
| 93565_primary Th1_resting dy 4-6 in IL-2 | 5.37 |
| 93566_primary Th2_resting dy 4-6 in IL-2 | 3.12 |
| 93567_primary Tr1_resting dy 4-6 in IL-2 | 0 |
| 93351_CD45RA CD4 lymphocyte_anti-CD28/anti-CD3 | 11.19 |
| 93352_CD45RO CD4 lymphocyte_anti-CD28/anti-CD3 | 1.15 |
| 93251_CD8 Lymphocytes_anti-CD28/anti-CD3 | 0.91 |
| 93353_chronic CD8 Lymphocytes 2ry_resting dy 4-6 in | 0 |
| IL-2 | |
| 93574_chronic CD8 Lymphocytes 2ry_activated | 0.63 |
| CD3/CD28 | |
| 93354_CD4_none | 1.08 |
| 93252_Secondary Th1/Th2/Tr1_anti-CD95 CH11 | 0 |
| 93103_LAK cells_resting | 0.49 |
| 93788_LAK cells_IL-2 | 0 |
| 93787_LAK cells_IL-2 + IL-12 | 0.69 |
| 93789_LAK cells_IL-2 + IFN gamma | 1.05 |
| 93790_LAK cells_IL-2 + IL-18 | 0.29 |
| 93104_LAK cells_PMA/ionomycin and IL-18 | 0 |
| 93578_NK Cells IL-2_resting | 1.34 |
| 93109_Mixed Lymphocyte Reaction_Two Way MLR | 0.54 |
| 93110_Mixed Lymphocyte Reaction_Two Way MLR | 0.47 |
| 93111_Mixed Lymphocyte Reaction_Two Way MLR | 2.65 |
| 93112_Mononuclear Cells (PBMCs)_resting | 0 |
| 93113_Mononuclear Cells (PBMCs)_PWM | 1.32 |
| 93114_Mononuclear Cells (PBMCs)_PHA-L | 1.02 |
| 93249_Ramos (B cell)_none | 1.21 |
| 93250_Ramos (B cell)_ionomycin | 2.26 |
| 93349_B lymphocytes_PWM | 4.27 |
| 93350_B lymphoytes_CD40L and IL-4 | 1.36 |
| 92665_EOL-1 (Eosinophil)_dbcAMP differentiated | 7.23 |
| 93248_EOL-1 (Eosinophil)_dbcAMP/PMAionomycin | 3.02 |
| 93356_Dendritic Cells_none | 1.48 |
| 93355_Dendritic Cells_LPS 100 ng/ml | 0.69 |
| 93775_Dendritic Cells_anti-CD40 | 0.5 |
| 93774_Monocytes_resting | 0.52 |
| 93776_Monocytes_LPS 50 ng/ml | 0 |
| 93581_Macrophages_resting | 1.29 |
| 93582_Macrophages_LPS 100 ng/ml | 1.75 |
| 93098_HUVEC (Endothelial)_none | 2.29 |
| 93099_HUVEC (Endothelial)_starved | 9.02 |
| 93100_HUVEC (Endothelial)_IL-1b | 1.16 |
| 93779_HUVEC (Endothelial)_IFN gamma | 1.41 |
| 93102_HUVEC (Endothelial)_TNF alpha + IFN gamma | 0.83 |
| 93101_HUVEC (Endothelial)_TNF alpha + IL4 | 1.12 |
| 93781_HUVEC (Endothelial)_IL-11 | 3 |
| 93583_Lung Microvascular Endothelial Cells_none | 0.77 |
| 93584_Lung Microvascular Endothelial Cells_TNFa (4 ng/ml) | 0.53 |
| and IL1b (1 ng/ml) | |
| 92662_Microvascular Dermal endothelium_none | 1.14 |
| 92663_Microsvasular Dermal endothelium_TNFa (4 ng/ml) | 1.03 |
| and IL1b (1 ng/ml) | |
| 93773_Bronchial epithelium_TNFa (4 ng/ml) and IL1b | 0 |
| (1 ng/ml)** | |
| 93347_Small Airway Epithelium_none | 0.39 |
| 93348_Small Airway Epithelium_TNFa (4 ng/ml) and | 0.53 |
| IL1b (1 ng/ml) | |
| 92668_Coronery Artery SMC_resting | 5.75 |
| 92669_Coronery Artery SMC_TNFa (4 ng/ml) and IL1b | 2.32 |
| (1 ng/ml) | |
| 93107_astrocytes_resting | 2.74 |
| 93108_astrocytes_TNFa (4 ng/ml) and IL1b (1 ng/ml) | 0 |
| 92666_KU-812 (Basophil)_resting | 6.79 |
| 92667_KU-812 (Basophil)_PMA/ionoycin | 8.42 |
| 93579_CCD1106 (Keratinocytes)_none | 1.58 |
| 93580_CCD1106 (Keratinocytes)_TNFa and IFNg** | 1.44 |
| 93791_Liver Cirrhosis | 4.18 |
| 93792_Lupus Kidney | 1.85 |
| 93577_NCI-H292 | 39.5 |
| 93358_NCI-H292_IL-4 | 38.96 |
| 93360_NCI-H292_IL-9 | 65.52 |
| 93359_NCI-H292_IL-13 | 37.11 |
| 93357_NCI-H292_IFN gamma | 31.86 |
| 93777_HPAEC_- | 0.48 |
| 93778_HPAEC_IL-1 beta/TNA alpha | 1.23 |
| 93254_Normal Human Lung Fibroblast_none | 42.34 |
| 93253_Normal Human Lung Fibroblast_TNFa (4 ng/ml) | 17.8 |
| and IL-1b (1 ng/ml) | |
| 93257_Normal Human Lung Fibroblast_IL-4 | 100 |
| 93256_Normal Human Lung Fibroblast_IL-9 | 72.7 |
| 93255_Normal Human Lung Fibroblast_IL-13 | 60.71 |
| 93258_Normal Human Lung Fibroblast_IFN gamma | 81.79 |
| 93106_Dermal Fibroblasts CCD1070_resting | 76.84 |
| 93361_Dermal Fibroblasts CCD1070_TNF alpha 4 ng/ml | 30.15 |
| 93105_Dermal Fibroblasts CCD1070_IL-1 beta 1 ng/ml | 38.16 |
| 93772_dermal fibroblast_IFN gamma | 34.15 |
| 93771_dermal fibroblast_IL-4 | 80.66 |
| 93259_IBD Colitis 1** | 0 |
| 93260_IBD Colitis 2 | 0.29 |
| 93261_IBD Crohns | 1.41 |
| 735010_Colon_normal | 35.6 |
| 735019_Lung_none | 11.03 |
| 64028-1_Thymus_none | 5.75 |
| 64030-1_Kidney_none | 9.67 |

### Example 4

### Expression Profiling of NOV6 (CG53980-01 or AL031704 A)

Panel 1.3 (Table 40): The profile was generated from a panel of 37 normal human tissues and 59 human cancer cell lines using specific gene probe and primer sets (Ag547). This gene is highly expressed in normal testes, placenta and colorectal tissue.

Panel 4D (Table 41): The profile was generated from a panel of several human cell lines that were either untreated or treated with a wide variety factors which modulate the immune response. This panel shows that the normal colon expresses high levels of this transcript whereas three different inflammatory bowel disease tissues did not.

| | |
|---|---|
| Probe Name: Ag547 | |
| Primers Sequences TM Length | |
| Start Position | |
| Forward | |
| 5′-TGACTGCTGCCCACTGCA-3′ | (SEQ ID NO.: 69) |
| Probe | |
| TET-5′-CACCGACCCGTCCATCTACCGGAT- | (SEQ ID NO.: 70) |
| 3′-TAMRA | |
| Reverse | |
| 5′-GAGATACACGTCCCCAGCGT-3′ | (SEQ ID NO.: 71) |

TABLE 40

| panel 1.3 ag547 | |
|---|---|
| Liver adenocarcinoma | 0 |
| Heart (fetal) | 0 |
| Pancreas | 0 |
| Pancreatic ca. CAPAN 2 | 0 |
| Adrenal gland | 0 |
| Thyroid | 0 |
| Salivary gland | 0 |
| Pituitary gland | 0 |
| Brain (fetal) | 0 |
| Brain (whole) | 0 |
| Brain (amygdala) | 0 |
| Brain (cerebellum) | 0 |
| Brain (hippocampus) | 0 |
| Brain (thalamus) | 0 |
| Cerebral Cortex | 0 |
| Spinal cord | 0 |
| CNS ca. (glio/astro) U87-MG | 0 |
| CNS ca. (glio/astro) U-118-MG | 0 |
| CNS ca. (astro) SW1783 | 0 |
| CNS ca.* (neuro; met) SK-N-AS | 0 |
| CNS ca. (astro) SF-539 | 0 |
| CNS ca. (astro) SNB-75 | 0 |
| CNS ca. (glio) SNB-19 | 0 |
| CNS ca. (glio) U251 | 0 |
| CNS ca. (glio) SF-295 | 0 |
| Heart | 0 |
| Skeletal muscle | 0 |
| Bone marrow | 0 |
| Thymus | 0 |
| Spleen | 0 |
| Lymph node | 0 |
| Colorectal | 41.87 |
| Stomach | 0 |
| Small intestine | 0 |
| Colon ca. SW480 | 0 |
| Colon ca.* (SW480 met)SW620 | 0 |
| Colon ca. HT29 | 28.93 |
| Colon ca. HCT-116 | 0 |
| Colon ca. CaCo-2 | 0 |
| 83219 CC Well to Mod Diff (ODO3866) | 0 |
| Colon ca. HCC-2998 | 0 |
| Gastric ca.* (liver met) NCI-N87 | 0 |
| Bladder | 0 |
| Trachea | 0 |
| Kidney | 0 |
| Kidney (fetal) | 0 |
| Renal ca. 786-0 | 0 |
| Renal ca. A498 | 0 |
| Renal ca. RXF 393 | 0 |
| Renal ca. ACHN | 0 |
| Renal ca. UO-31 | 0 |
| Renal ca. TK-10 | 0 |
| Liver | 0 |
| Liver (fetal) | 0 |
| Liver ca. (hepatoblast) HepG2 | 0 |
| Lung | 0 |
| Lung (fetal) | 0 |
| Lung ca. (small cell) LX-1 | 0 |
| Lung ca. (small cell) NCI-H69 | 0 |
| Lung ca. (s. cell var.) SHP-77 | 0 |
| Lung ca. (large cell)NCI-H460 | 0 |
| Lung ca. (non-sm. cell) A549 | 0 |
| Lung ca. (non-s. cell) NCI-H23 | 0 |
| Lung ca (non-s. cell) HOP-62 | 0 |
| Lung ca. (non-s. cl) NCI-H522 | 0 |
| Lung ca. (squam.) SW 900 | 0 |
| Lung ca. (squam.) NCI-H596 | 0 |
| Mammary gland | 0 |
| Breast ca.* (pl. effusion) MCF-7 | 0 |
| Breast ca.* (pl. ef) MDA-MB-231 | 0 |
| Breast ca.* (pl. effusion) T47D | 0 |
| Breast ca. BT-549 | 53.27 |
| Breast ca. MDA-N | 0 |
| Ovary | 0 |
| Ovarian ca. OVCAR-3 | 0 |
| Ovarian ca. OVCAR-4 | 0 |
| Ovarian ca. OVCAR-5 | 0 |
| Ovarian ca. OVCAR-8 | 0 |
| Ovarian ca. IGROV-1 | 0 |
| Ovarian ca.* (ascites) SK-OV-3 | 0 |
| Uterus | 0 |
| Plancenta | 52.86 |
| Prostate | 0 |
| Prostate ca.* (bone met)PC-3 | 0 |
| Testis | 100 |
| Melanoma Hs688(A).T | 0 |
| Melanoma* (met) Hs688(B).T | 0 |
| Melanoma UACC-62 | 0 |
| Melanoma M14 | 0 |
| Melanoma LOX IMVI | 0 |
| Melanoma* (met) SK-MEL-5 | 0 |
| Adipose | 0 |

TABLE 41

| panel 4D ag547 | |
|---|---|
| 93768_Secondary Th1_anti-CD28/anti-CD3 | 0 |
| 93769_Secondary Th2_anti-CD28/anti-CD3 | 0 |
| 93770_Secondary Tr1_anti-CD28/anti-CD3 | 0 |
| 93573_Secondary Th1_resting day 4-6 in IL-2 | 0 |
| 93572_Secondary Th2_resting day 4-6 in IL-2 | 0 |
| 93571_Secondary Tr1_resting day 4-6 in IL-2 | 0 |
| 93568_primary Th1_anti-CD28/anti-CD3 | 0 |
| 93569_primary Th2_anti-CD28/anti-CD3 | 0 |
| 93570_primary Tr1_anti-CD28/anti-CD3 | 0 |
| 93565_primary Th1_resting dy 4-6 in IL-2 | 0 |
| 93566_primary Th2_resting dy 4-6 in IL-2 | 0 |
| 93567_primary Tr1_resting dy 4-6 in IL-2 | 0 |
| 93351_CD45RA CD4 lymphocyte_anti-CD28/anti-CD3 | 0 |
| 93352_CD45RO CD4 lymphocyte_anti-CD28/anti-CD3 | 0 |
| 93251_CD8 Lymphocytes_anti-CD28/anti-CD3 | 0 |
| 93353_chronic CD8 Lymphocytes 2ry_resting dy 4-6 in IL-2 | 0 |
| 93574_chronic CD8 Lymphocytes 2ry_activated CD3/CD28 | 0 |
| 93354_CD4_none | 0 |
| 93252_Secondary Th1/Th2/Tr1_anti-CD95 CH11 | 0 |
| 93103_LAK cells_resting | 0 |
| 93788_LAK cells_IL-2 | 0 |
| 93787_LAK cells_IL-2 + IL-12 | 14.66 |
| 93789_LAK cells_IL-2 + IFN gamma | 0 |
| 93790_LAK cells_IL-2 + IL-18 | 0 |
| 93104_LAK cells_PMA/ionomycin and IL-18 | 0 |
| 93578_NK Cells IL-2_resting | 0 |
| 93109_Mixed Lymphocyte Reaction_Two Way MLR | 16.96 |
| 93110_Mixed Lymphocyte Reaction_Two Way MLR | 0 |
| 93111_Mixed Lymphocyte Reaction_Two Way MLR | 0 |
| 93112_Mononuclear Cells (PBMCs)_resting | 0 |
| 93113_Mononuclear Cells (PBMCs)_PWM | 0 |
| 93114_Mononuclear Cells (PBMCs)_PHA-L | 0 |
| 93249_Ramos (B cell)_none | 0 |
| 93250_Ramos (B cell)_ionomycin | 0 |
| 93349_B lymphocytes_PWM | 0 |
| 93350_B lymphoytes_CD40L and IL-4 | 14.46 |
| 92665_EOL-1 (Eosinophil)_dbcAMP differentiated | 0 |
| 93248_EOL-1 (Eosinophil)_dbcAMP/PMAionomycin | 0 |
| 93356_Dendritic Cells_none | 0 |
| 93355_Dendritic Cells_LPS 100 ng/ml | 0 |
| 93775_Dendritic Cells_anti-CD40 | 17.8 |
| 93774_Monocytes_resting | 0 |
| 93776_Monocytes_LPS 50 ng/ml | 8.9 |
| 93581_Macrophages_resting | 0 |
| 93582_Macrophages_LPS 100 ng/ml | 0 |
| 93098_HUVEC (Endothelial)_none | 0 |
| 93099_HUVEC (Endothelial)_starved | 0 |
| 93100_HUVEC (Endothelial)_IL-1b | 0 |
| 93779_HUVEC (Endothelial)_IFN gamma | 0 |
| 93102_HUVEC (Endothelial)_TNF alpha + IFN gamma | 0 |
| 93101_HUVEC (Endothelial)_TNF alpha + IL4 | 0 |
| 93781_HUVEC (Endothelial)_IL-11 | 0 |
| 93583_Lung Microvascular Endothelial Cells_none | 0 |
| 93584_Lung Microvascular Endothelial Cells_TNFa (4 ng/ml) | 0 |
| and IL1b (1 ng/ml) | |
| 92662_Microvascular Dermal endothelium_none | 0 |
| 92663_Microsvasular Dermal endothelium_TNF | 0 |
| a (4 ng/ml) and IL1b (1 ng/ml) | |
| 93773_Bronchial epithelium_TNFa (4 ng/ml) and | 0 |
| IL1b (1 ng/ml)** | |
| 93347_Small Airway Epithelium_none | 0 |
| 93348_Small Airway Epithelium_TNFa (4 ng/ml) and | 0 |
| IL1b (1 ng/ml) | |
| 92668_Coronery Artery SMC_resting | 0 |
| 92669_Coronery Artery SMC_TNFa (4 ng/ml) and | 0 |
| IL1b (1 ng/ml) | |
| 93107_astrocytes_resting | 0 |
| 93108_astrocytes_TNFa (4 ng/ml) and IL1b (1 ng/ml) | 0 |
| 92666_KU-812 (Basophil)_resting | 0 |
| 92667_KU-812 (Basophil)_PMA/ionoycin | 10.88 |
| 93579_CCD1106 (Keratinocytes)_none | 0 |
| 93580_CCD1106 (Keratinocytes)_TNFa and IFNg** | 0 |
| 93791_Liver Cirrhosis | 13.12 |
| 93792_Lupus Kidney | 0 |
| 93577_NCI-H292 | 0 |
| 93358_NCI-H292_IL-4 | 0 |
| 93360_NCI-H292_IL-9 | 16.61 |
| 93359_NCI-H292_IL-13 | 0 |
| 93357_NCI-H292_IFN gamma | 0 |
| 93777_HPAEC_- | 10.37 |
| 93778_HPAEC_IL-1 beta/TNA alpha | 0 |
| 93254_Normal Human Lung Fibroblast_none | 0 |
| 93253_Normal Human Lung Fibroblast_TNFa (4 ng/ml) | 0 |
| and IL-1b(1 ng/ml) | |
| 93257_Normal Human Lung Fibroblast_IL-4 | 0 |
| 93256_Normal Human Lung Fibroblast_IL-9 | 0 |
| 93255_Normal Human Lung Fibroblast_IL-13 | 0 |
| 93258_Normal Human Lung Fibroblast_IFN gamma | 0 |
| 93106_Dermal Fibroblasts CCD1070_resting | 3.74 |
| 93361_Dermal Fibroblasts CCD1070_TNF alpha 4 ng/ml | 0 |
| 93105_Dermal Fibroblasts CCD1070_IL-1 beta 1 ng/ml | 0 |
| 93772_dermal fibroblast_IFN gamma | 0 |
| 93771_dermal fibroblast_IL-4 | 0 |
| 93259_IBD Colitis 1** | 0 |
| 93260_IBD Colitis 2 | 0 |
| 93261_IBD Crohns | 0 |
| 735010_Colon_normal | 100 |
| 735019_Lung_none | 33.92 |
| 64028-1_Thymus_none | 0 |
| 64030-1_Kidney_none | 0 |

### Example 5

### Expression Profiling of NOV11 (CG54565-05)

Panel 4D (Table 42): The CG54656-05 transcript is up regulated in three different epithelial cell types after treatment with inflammatory cytokines. Two cell lines originate from lung tissue, the NCI H292 airway cell line and lung microvascular endothelial cells. Human umbilical vein epithelial cells (HUVEC) also up regulate expression of this transcript upon activation.

| Probe Name: Ag1599 | | | |
|---|---|---|---|
| Primers Sequences TM Length Start Position | | | |
| Forward | 5′-CTCAAGTACCACACGGTCTCAT-3′ | (SEQ ID NO.: 72) | 59.1 22 400 |
| Probe | TET-5′-CCGCACCCGGAAAGTCATTGTAAGT-3′-TAMRA | (SEQ ID NO.: 73) | 69.8 25 429 |
| Reverse | 5′-TCAGGAAGCAGGTGATGTAAAC-3′ | (SEQ ID NO.: 74) | 59.2 22 454 |

TABLE 42

| panel 4D | 4dtm4722_ag1599 |
|---|---|
| Secondary Th1 act | 0 |
| Secondary Th2 act | 0 |
| Secondary Tr1 act | 0 |
| Secondary Th1 rest | 0 |
| Secondary Th2 rest | 0 |
| Secondary Tr1 rest | 0 |
| PrimaryTh1 act | 0 |
| Primary Th2 act | 0 |
| Primary Tr1 act | 0 |
| Primary Th1 rest | 0 |
| Primary Th2 rest | 0 |
| Primary Tr1 rest | 13.77 |
| CD45RA CD4 lymphocyte act | 0 |
| CD45RO CD4 lymphocyte act | 40.61 |
| CD8 lymphocyte act | 0 |
| Secondary CD8 lymphocyte rest | 0 |
| Secondary CD8 lymphocyte act | 0 |
| CD4 lymphocyte none | 0 |
| 2ry Th1/Th2/Tr1_anti-CD95 CH11 | 0 |
| LAK cells rest | 0 |
| LAK cells IL-2 | 0 |
| LAK cells IL-2 + IL-12 | 0 |
| LAK cells IL-2 + IFN gamma | 0 |
| LAK cells IL-2 + IL-18 | 0 |
| LAK cells PMA/ionomycin | 0 |
| NK Cells IL-2 rest | 0 |
| Two Way MLR 3 day | 0 |
| Two Way MLR 5 day | 0 |
| Two Way MLR 7 day | 0 |
| PBMC rest | 0 |
| PBMC PWM | 0 |
| PBMC PHA-L | 0 |
| Ramos (B cell) none | 0 |
| Ramos (B cell) ionomycin | 0 |
| B lymphocytes PWM | 0 |
| B lymphocytes CD40L and IL-4 | 0 |
| EOL-1 dbcAMP | 0 |
| EOL-1 dbcAMP PMA/ionomycin | 0 |
| Dendritic cells none | 0 |
| Dendritic cells LPS | 0 |
| Dendritic cells anti-CD40 | 0 |
| Monocytes rest | 0 |
| Monocytes LPS | 0 |
| Macrophages rest | 0 |
| Macrophages LPS | 0 |
| HUVEC none | 0 |
| HUVEC starved | 0 |
| HUVEC IL-1beta | 0 |
| HUVEC IFN gamma | 0 |
| HUVEC TNF alpha + IFN gamma | 30.35 |
| HUVEC TNF alpha + IL4 | 0 |
| HUVEC IL-11 | 0 |
| Lung Microvascular EC none | 0 |
| Lung Microvascular EC TNF alpha + IL-1beta | 27.93 |
| Microvascular Dermal EC none | 0 |
| Microsvasular Dermal EC TNF alpha + IL-1beta | 0 |
| Bronchial epithelium TNF alpha + IL1beta | 0 |
| Small airway epithelium none | 0 |
| Small airway epithelium TNF alpha + IL-1beta | 0 |
| Coronery artery SMC rest | 0 |
| Coronery artery SMC TNF alpha + IL-1beta | 0 |
| Astrocytes rest | 0 |
| Astrocytes TNF alpha + IL-1beta | 0 |
| KU-812 (Basophil) rest | 0 |
| KU-812 (Basophil) PMA/ionomycin | 0 |
| CCD1106 (Keratinocytes) none | 0 |
| CCD1106 (Keratinocytes) TNF alpha + IL- | 0 |
| 1beta | |
| Liver cirrhosis | 29.12 |
| Lupus kidney | 0 |
| NCI-H292 none | 0 |
| NCI-H292 IL-4 | 0 |
| NCI-H292 IL-9 | 0 |
| NCI-H292 IL-13 | 0 |
| NCI-H292 IFN gamma | 12.85 |
| HPAEC none | 0 |
| HPAEC TNF alpha + IL-1 beta | 0 |
| Lung fibroblast none | 0 |
| Lung fibroblast TNF alpha + IL-1 beta | 0 |
| Lung fibroblast IL-4 | 0 |
| Lung fibroblast IL-9 | 0 |
| Lung fibroblast IL-13 | 0 |
| Lung fibroblast IFN gamma | 0 |
| Dermal fibroblast CCD1070 rest | 100 |
| Dermal fibroblast CCD1070 TNF alpha | 0 |
| Dermal fibroblast CCD1070 IL-1 beta | 0 |
| Dermal fibroblast IFN gamma | 0 |
| Dermal fibroblast IL-4 | 0 |
| IBD Colitis 1 | 0 |
| IBD Colitis 2 | 21.02 |
| IBD Crohn's | 0 |
| Colon | 17.19 |
| Lung | 0 |
| Thymus | 0 |
| Kidney | 0 |

### Example 6

### Expression Profiling of NOV8 (CG58604 or 416_d_{—}14 A).

TaqMan Expression Profile of CG58604 Transcript:

Panel 1.1 (Tables 43 and 45): There is very low expression of this transcript in most normal tissues with the exception of the brain. The expression of this transcript in the normal lung is very low.

Panel 4D (Tables 44 and 46): Lung fibroblast expression of CG58604 is up highly regulated by IL-13. This transcript is also expressed on IL-4 treated dermal fibroblasts.

Probe Name: Ag552

| | |
|---|---|
| Primers | Sequences |

| | | |
|---|---|---|
| Forward | 5′-GGAAGCTGACCGACCAGAAC-3′ | (SEQ ID NO.: 75) |
| Probe | FAM-5′-AGCCCATCCCTAGAGCCTTCATGTACTCA-3′-TAMRA | (SEQ ID NO.: 76) |
| Reverse | 5′-ATTTCCCACCTGCCTAGTGACA-3′ | (SEQ ID NO.: 77)+TZ,1/44 |

[image] TABLE 45

| Panel 1.1 ag552 | 1.1tm699f_ag552 |
|---|---|
| Adipose | 14.76 |
| Adrenal gland | 5.44 |
| Bladder | 6.93 |
| Brain (amygdala) | 3.79 |
| Brain (cerebellum) | 100 |
| Brain (hippocampus) | 12.94 |
| Brain (substantia nigra) | 19.75 |
| Brain (thalamus) | 7.86 |
| Cerebral Cortex | 9.34 |
| Brain (fetal) | 36.35 |
| Brain (whole) | 6.47 |
| CNS ca. (glio/astro) U-118-MG | 2.78 |
| CNS ca. (astro) SF-539 | 4.09 |
| CNS ca. (astro) SNB-75 | 2.94 |
| CNS ca. (astro) SW1783 | 0.24 |
| CNS ca. (glio) U251 | 2.37 |
| CNS ca. (glio) SF-295 | 4.7 |
| CNS ca. (glio) SNB-19 | 4.36 |
| CNS ca. (glio/astro) U87-MG | 3.19 |
| CNS ca.* (neuro; met) SK-N-AS | 8.3 |
| Mammary gland | 1.15 |
| Breast ca. BT-549 | 1.41 |
| Breast ca. MDA-N | 3.35 |
| Breast ca.* (pl. effusion) T47D | 4.07 |
| Breast ca.* (pl. effusion) MCF-7 | 0 |
| Breast ca.* (pl. ef) MDA-MB-231 | 2.09 |
| Small intestine | 3.82 |
| Colorectal | 0.37 |
| Colon ca. HT29 | 0.36 |
| Colon ca. CaCo-2 | 0 |
| Colon ca. HCT-15 | 1.13 |
| Colon ca. HCT-116 | 0.21 |
| Colon ca. HCC-2998 | 1.1 |
| Colon ca. SW480 | 0.44 |
| Colon ca.* (SW480 met)SW620 | 1.81 |
| Stomach | 1.91 |
| Gastric ca.* (liver met) NCI-N87 | 4.48 |
| Heart | 13.21 |
| Fetal Skeletal | 2.01 |
| Skeletal muscle | 6.79 |
| Endothelial cells | 3.77 |
| Endothelial cells (treated) | 0 |
| Kidney | 9.88 |
| Kidney (fetal) | 4.74 |
| Renal ca. 786-0 | 2.05 |
| Renal ca. A498 | 1.07 |
| Renal ca. ACHN | 5.01 |
| Renal ca. TK-10 | 11.58 |
| Renal ca. UO-31 | 7.97 |
| Renal ca. RXF 393 | 3.77 |
| Liver | 2.26 |
| Liver (fetal) | 0.5 |
| Liver ca. (hepatoblast) HepG2 | 0 |
| Lung | 2.29 |
| Lung (fetal) | 1.49 |
| Lung ca (non-s. cell) HOP-62 | 34.87 |
| Lung ca. (large cell)NCI-H460 | 4.74 |
| Lung ca. (non-s. cell) NCI-H23 | 2.88 |
| Lung ca. (non-s. cl) NCI-H522 | 0.71 |
| Lung ca. (non-sm. cell) A549 | 16.49 |
| Lung ca. (s. cell var.) SHP-77 | 2.26 |
| Lung ca. (small cell) LX-1 | 4.07 |
| Lung ca. (small cell) NCI-H69 | 11.34 |
| Lung ca. (squam.) SW 900 | 1.63 |
| Lung ca. (squam.) NCI-H596 | 15.71 |
| Lymph node | 4.3 |
| Spleen | 0 |
| Thymus | 2.24 |
| Ovary | 0.62 |
| Ovarian ca. IGROV-1 | 0.68 |
| Ovarian ca. OVCAR-3 | 1.17 |
| Ovarian ca. OVCAR-4 | 0 |
| Ovarian ca. OVCAR-5 | 7.13 |
| Ovarian ca. OVCAR-8 | 5.63 |
| Ovarian ca.* (ascites) SK-OV-3 | 1.58 |
| Pancreas | 6.29 |
| Pancreatic ca. CAPAN 2 | 3.33 |
| Pituitary gland | 7.64 |
| Plancenta | 4.9 |
| Prostate | 4.45 |
| Prostate ca.* (bone met)PC-3 | 8.84 |
| Salavary gland | 5.08 |
| Trachea | 2.3 |
| Spinal cord | 4.87 |
| Testis | 1.71 |
| Thyroid | 2.61 |
| Uterus | 5.11 |
| Melanoma M14 | 9.02 |
| Melanoma LOX IMVI | 1.49 |
| Melanoma UACC-62 | 23 |
| Melanoma SK-MEL-28 | 28.92 |
| Melanoma* (met) SK-MEL-5 | 4.33 |
| Melanoma Hs688(A).T | 3.98 |
| Melanoma* (met) Hs688(B).T | 6.29 |

TABLE 46

| Panel 4D ag552 | 4dtm4830f_ag552 | 4dx4tm5143f_ag552_b1 |
|---|---|---|
| 93768_Secondary Th1_anti-CD28/anti-CD3 | 0 | 4.42 |
| 93769_Secondary Th2_anti-CD28/anti-CD3 | 0 | 5.52 |
| 93770_Secondary Tr1_anti-CD28/anti-CD3 | 0 | 8.55 |
| 93573_Secondary Th1_resting day 4-6 in IL-2 | 19.75 | 3.74 |
| 93572_Secondary Th2_resting day 4-6 in IL-2 | 0 | 3.99 |
| 93571_Secondary Tr1_resting day 4-6 in IL-2 | 0 | 7.71 |
| 93568_primary Th1_anti-CD28/anti-CD3 | 0 | 8 |
| 93569_primary Th2_anti-CD28/anti-CD3 | 0 | 7.9 |
| 93570_primary Tr1_anti-CD28/anti-CD3 | 0 | 18.09 |
| 93565_primary Th1_resting dy 4-6 in IL-2 | 0 | 22.26 |
| 93566_primary Th2_resting dy 4-6 in IL-2 | 0 | 21.49 |
| 93567_primary Tr1_resting dy 4-6 in IL-2 | 0 | 13.65 |
| 93351_CD45RA CD4 lymphocyte_anti- | 0 | 14.02 |
| CD28/anti-CD3 | | |
| 93352_CD45RO CD4 lymphocyte_anti- | 0 | 11.44 |
| CD28/anti-CD3 | | |
| 93251_CD8 Lymphocytes_anti-CD28/anti-CD3 | 14.06 | 15.03 |
| 93353_chronic CD8 Lymphocytes 2ry_resting dy | 0 | 10.1 |
| 4-6 in IL-2 | | |
| 93574_chronic CD8 Lymphocytes 2ry_activated | 0 | 11.13 |
| CD3/CD28 | | |
| 93354_CD4_none | 0 | 22 |
| 93252_Secondary Th1/Th2/Tr1_anti-CD95 CH11 | 0 | 22.66 |
| 93103_LAK cells_resting | 0 | 12.75 |
| 93788_LAK cells_IL-2 | 0 | 15.64 |
| 93787_LAK cells_IL-2 + IL-12 | 0 | 10.71 |
| 93789_LAK cells_IL-2 + IFN gamma | 0 | 34.75 |
| 93790_LAK cells_IL-2 + IL-18 | 0 | 24.21 |
| 93104_LAK cells_PMA/ionomycin and IL-18 | 0 | 5.46 |
| 93578_NK Cells IL-2_resting | 0 | 15.92 |
| 93109_Mixed Lymphocyte Reaction_Two Way | 0 | 24.93 |
| MLR | | |
| 93110_Mixed Lymphocyte Reaction_Two Way | 0 | 4.72 |
| MLR | | |
| 93111_Mixed Lymphocyte Reaction_Two Way | 0 | 5.17 |
| MLR | | |
| 93112_Mononuclear Cells (PBMCs)_resting | 0 | 16.01 |
| 93113_Mononuclear Cells (PBMCs)_PWM | 0 | 45.74 |
| 93114_Mononuclear Cells (PBMCs)_PHA-L | 0 | 17.56 |
| 93249_Ramos (B cell)_none | 0 | 0 |
| 93250_Ramos (B cell)_ionomycin | 0 | 0 |
| 93349_B lymphocytes_PWM | 0 | 33.76 |
| 93350_B lymphoytes_CD40L and IL-4 | 0 | 24.37 |
| 92665_EOL-1 (Eosinophil)_dbcAMP | 0 | 3.2 |
| differentiated | | |
| 93248_EOL-1 | 0 | 9.34 |
| (Eosinophil)_dbcAMP/PMAionomycin | | |
| 93356_Dendritic Cells_none | 0 | 5.54 |
| 93355_Dendritic Cells_LPS 100 ng/ml | 0 | 5.13 |
| 93775_Dendritic Cells_anti-CD40 | 0 | 0.91 |
| 93774_Monocytes_resting | 0 | 8.66 |
| 93776_Monocytes_LPS 50 ng/ml | 0 | 23.67 |
| 93581_Macrophages_resting | 0 | 9.01 |
| 93582_Macrophages_LPS 100 ng/ml | 0 | 14.6 |
| 93098_HUVEC (Endothelial)_none | 0 | 13.86 |
| 93099_HUVEC (Endothelial)_starved | 0 | 100 |
| 93100_HUVEC (Endothelial)_IL-1b | 0 | 10.46 |
| 93779_HUVEC (Endothelial)_IFN gamma | 0 | 13.95 |
| 93102_HUVEC (Endothelial)_TNF alpha + IFN | 0 | 11.95 |
| gamma | | |
| 93101_HUVEC (Endothelial)_TNF alpha + IL4 | 0 | 8.99 |
| 93781_HUVEC (Endothelial)_IL-11 | 0 | 8.53 |
| 93583_Lung Microvascular Endothelial | 0 | 16.62 |
| Cells_none | | |
| 93584_Lung Microvascular Endothelial | 0 | 11.25 |
| Cells_TNFa (4 ng/ml) and IL1b (1 ng/ml) | | |
| 92662_Microvascular Dermal endothelium_none | 0 | 32.23 |
| 92663_Microsvasular Dermal endothelium_TNFa | 0 | 19.86 |
| (4 ng/ml) and IL1b(1 ng/ml) | | |
| 93773_Bronchial epithelium_TNFa (4 ng/ml) and | 0 | 6.14 |
| IL1b (1 ng/ml)** | | |
| 93347_Small Airway Epithelium_none | 0 | 1.75 |
| 93348_Small Airway Epithelium_TNFa (4 ng/ml) | 0 | 3.14 |
| and IL1b (1 ng/ml) | | |
| 92668_Coronery Artery SMC_resting | 0 | 6.27 |
| 92669_Coronery Artery SMC_TNFa (4 ng/ml) and | 0 | 3.37 |
| IL1b (1 ng/ml) | | |
| 93107_astrocytes_resting | 0 | 8.3 |
| 93108_astrocytes_TNFa (4 ng/ml) and IL1b (1 ng/ml) | 0 | 2.09 |
| 92666_KU-812 (Basophil)_resting | 0 | 0 |
| 92667_KU-812 (Basophil)_PMA/ionoycin | 0 | 0.45 |
| 93579_CCD1106 (Keratinocytes)_none | 0 | 3.61 |
| 93580_CCD1106 (Keratinocytes)_TNFa and | 0 | 2.71 |
| IFNg** | | |
| 93791_Liver Cirrhosis | 0 | 12.68 |
| 93792_Lupus Kidney | 0 | 6.27 |
| 93577_NCI-H292 | 0 | 0 |
| 93358_NCI-H292_IL-4 | 0 | 0.52 |
| 93360_NCI-H292_IL-9 | 0 | 0 |
| 93359_NCI-H292_IL-13 | 0 | 0 |
| 93357_NCI-H292_IFN gamma | 0 | 0 |
| 93777_HPAEC_- | 0 | 15.91 |
| 93778_HPAEC_IL-1 beta/TNA alpha | 0 | 20.2 |
| 93254_Normal Human Lung Fibroblast_none | 0 | 5.28 |
| 93253_Normal Human Lung Fibroblast_TNFa (4 ng/ml) | 12.85 | 6.62 |
| and IL-1b (1 ng/ml) | | |
| 93257_Normal Human Lung Fibroblast_IL-4 | 0 | 3.44 |
| 93256_Normal Human Lung Fibroblast_IL-9 | 0 | 4.77 |
| 93255_Normal Human Lung Fibroblast_IL-13 | 100 | 4.56 |
| 93258_Normal Human Lung Fibroblast_IFN | 0 | 5.17 |
| gamma | | |
| 93106_Dermal Fibroblasts CCD1070_resting | 0 | 21.94 |
| 93361_Dermal Fibroblasts CCD1070_TNF alpha | 0 | 33.7 |
| 4 ng/ml | | |
| 93105_Dermal Fibroblasts CCD1070_IL-1 beta 1 ng/ml | 0 | 84.47 |
| 93772_dermal fibroblast_IFN gamma | 0 | 7.42 |
| 93771_dermal fibroblast_IL-4 | 74.74 | 4.83 |
| 93259_IBD Colitis 1** | 0 | 2.52 |
| 93260_IBD Colitis 2 | 0 | 1.64 |
| 93261_IBD Crohns | 24.83 | 0.62 |
| 735010_Colon_normal | 0 | 11.96 |
| 735019_Lung_none | 0 | 5.73 |
| 64028-1_Thymus_none | 16.84 | 13.44 |
| 64030-1_Kidney_none | 0 | 51.45 |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated nucleic acid molecule comprising a nucleic acid sequence encoding a polypeptide comprising an amino acid sequence selected from the group consisting of:
a) a mature form of the amino acid sequence of SEQ ID NO: 20;
b) a variant of a mature form of the amino acid sequence of SEQ ID NO: 20 wherein any amino acid in the mature form of the chosen sequence is changed to a different amino acid, provided that no more than 15% of the amino acid residues in the sequence of the mature form are so changed;
c) the amino acid sequence of SEQ ID NO: 20;
d) a variant of the amino acid sequence of SEQ ID NO: 20, in which any amino acid specified in the chosen sequence is changed to a different amino acid, provided that no more than 15% of the amino acid residues in the sequence are so changed;
e) a nucleic acid fragment encoding at least a portion of a polypeptide comprising the amino acid sequence of SEQ ID NO: 20 or any variant of said polypeptide wherein any amino acid of the chosen sequence is changed to a different amino acid, provided that no more than 10% of the amino acid residues in the sequence are so changed; and
f) the complement of any of said nucleic acid molecules.

2. The nucleic acid molecule of claim 1, wherein the nucleic acid molecule comprises the nucleotide sequence of a naturally occurring allelic nucleic acid variant.

3. The nucleic acid molecule of claim 2 that encodes a variant polypeptide, wherein the variant polypeptide has the polypeptide sequence of a naturally occurring polypeptide variant.

4. The nucleic acid molecule of claim 1, wherein the nucleic acid molecule comprises a single nucleotide polymorphism encoding said variant polypeptide.

5. The nucleic acid molecule of claim 1, wherein said nucleic acid molecule comprises a nucleotide sequence selected from the group consisting of
a) the nucleotide sequence of SEQ ID NO: 19;
b) a nucleotide sequence wherein one or more nucleotides in the nucleotide sequence of SEQ ID NO: 19 is changed from that selected from the group consisting of the chosen sequence to a different nucleotide provided that no more than 15% of the nucleotides are so changed;
c) a nucleic acid fragment of the sequence of SEQ ID NO: 19; and
d) a nucleic acid fragment wherein one or more nucleotides in the nucleotide sequence of SEQ ID NO: 19 is changed from that selected from the group consisting of the chosen sequence to a different nucleotide provided that no more than 15% of the nucleotides are so changed.

6. The nucleic acid molecule of claim 1, wherein said nucleic acid molecule hybridizes under stringent conditions to the nucleotide sequence of SEQ ID NO: 19, or a complement of said nucleotide sequence.

7. The nucleic acid molecule of claim 1, wherein the nucleic acid molecule comprises a nucleotide sequence in which any nucleotide specified in the coding sequence of the chosen nucleotide sequence is changed from that selected from the group consisting of the chosen sequence to a different nucleotide provided that no more than 15% of the nucleotides in the chosen coding sequence are so changed, and an isolated second polynucleotide that is a complement of the first polynucleotide.

8. A vector comprising the nucleic acid molecule of claim 7.

9. The vector of claim 8, further comprising a promoter operably linked to said nucleic acid molecule.

10. A cell comprising the vector of claim 10.

11. A composition comprising the nucleic acid molecule of claim 1 and an acceptable carrier.

12. A kit comprising in one or more containers, the composition of claim 11.
